(19) United States
(12) Patent Application Publication
(10) Pub. No.: US 2025/0258170 A1
(43) Pub. Date: Aug. 14, 2025

(54) Proteins and T-Cells Involved in Chronic Inflammatory Diseases

(71) Applicant: Christian-Albrechts-Universität zu Kiel, Kiel (DE)

(72) Inventors: Elisa Rosati, Kiel (DE); Andre Franke, Kronshagen (DE); Petra Bacher, Kiel (DE); Ilgar Mamedov, Moscow (RU)

(73) Assignee: Christian-Albrechts-Universität zu Kiel, Kiel (DE)

(21) Appl. No.: 18/723,255

(22) PCT Filed: Dec. 22, 2022
(86) PCT No.: PCT/EP2022/087583

§ 371 Date: Jun. 21, 2024

(30) Foreign Application Priority Data
Dec. 22, 2021 (EP) 21217058.3

(51) Int. Cl. G01N 33/569; C07K 16/28; G01N 33/68

(52) U.S. Cl. CPC G01N 33/56972; C07K 16/2809; G01N 33/6893; G01N 2800/065

(57) ABSTRACT

A protein comprising an amino acid sequence according to SEQ ID NO: 1, wherein the amino acid in position 4 of SEQ ID NO: 1 is selected from the group consisting of N, S, R, T and I, preferably consisting of N, S and R and wherein the amino acid in position 5 of SEQ ID NO: 1 is selected from the group consisting of R, V, L, F, M, I, H, S, T, A, P and G, with the proviso that the amino acid sequence is not SEQ ID NO: 24.

### CROSS-REFERENCE TO RELATED APPLICATIONS

The present application is the U.S. National Stage of International Patent Application No. PCT/EP2022/087583, filed Dec. 22, 2022, and claims priority to European Patent Application No. 21217058.3, filed Dec. 22, 2021.

### SEQUENCE LISTING

The instant application contains a Sequence Listing which has been submitted electronically in XML format and is hereby incorporated by reference in its entirety. Said XML file, created on Jan. 27, 2025, is named “117948-0112_SL.xml” and is 1,078,193 bytes in size.

### FIELD

The present invention relates to proteins comprising an amino acid sequence according to a specific motif. The invention further relates to a nucleotide sequence encoding for such an amino acid sequence and a T-cell comprising the protein according to the invention. Further, the invention relates to the use of a protein according to the invention or a T-cell according to the invention or an mRNA according to the invention in the research for a medicament for treatment of a chronic inflammatory disease or in the development of an antibody directed against the protein and/or the T-cell and/or the mRNA according to the invention.

The invention further relates to an antibody directed against an epitope of the protein according to the invention.

### STATE OF THE ART

Dysregulated T cell reactions against intestinal antigens are considered to be a causal or driving factor for chronic inflammatory diseases, especially inflammatory bowel diseases (IBD). For IBD and for other inflammatory and autoimmune diseases, e.g. multiple sclerosis or diabetes, the excessive immune response may be directed against common, still unknown, disease-driving antigens. In genetically predisposed hosts, antigens derived from a dysbiotic microbiome may come into contact with T cells through the damaged intestinal epithelial barrier, which may lead to an uncontrolled and dysregulated immune response. As such, it may be possible to identify specific disease-associated T cells within the peripheral blood and/or at the intestinal inflammation site of IBD patients.

Although the two main forms of IBD, namely Crohn's disease (CD) and ulcerative colitis (UC) are often referred to as generally “IBD”, they are two independent diseases with different disease progression and characteristics. CD may develop in any part of the gastrointestinal tract, mostly in the small intestine, while UC develops in the colon only. Both diseases are T-cell mediated, different T cell subsets may but do not have to play a role in disease pathogenesis and progression. Markers distinguishing the two diseases exist but are limited, sometimes resulting in incorrect diagnosis. Further insights differentiating the two forms would help in better understanding, diagnosis and treatment of the diseases.

Most alpha/beta T cells are characterized by a unique T-cell receptor (TCR), formed by an alpha and beta chain. T cells recognize antigenic peptides presented by the major histocompatibility complex (MHC). Upon recognition of an antigen via the TCR, T cells may expand clonally, resulting in a population of cells with identical TCRs (“clonotypes”). The TCR repertoire, defined as the collection of unique TCRs, is extremely variable, with up to 10¹⁶ unique clonotypes present in a single individual. Therefore, the composition of the TCR repertoire, i.e. the diversity of different TCRs or the expansion of individual clonotypes, can provide important insights into disease-associated alterations of the T cell reaction.

In general, there is a need for the identification of disease-associated T-cell clonotypes in chronic inflammatory disease patients, especially in IBD patients, which could be an important step towards (i) understanding the contribution of TCR repertoire changes to diseases' pathophysiology; (ii) the identification of pathogenic T cells of interest; (iii) the identification of pathogenic T cells' cognate antigens and (iv) the development of new diagnostic and therapeutic strategies. Previous studies identified expanded clonotypes in IBD intestinal samples compared to blood samples and correlated certain TCRs with disease recurrence after surgery.

In view of the state of the art, it was an object of the present invention to provide means for research and development of diagnostic and therapeutic strategies for chronic inflammatory diseases, especially for IBD.

### DESCRIPTION

In a first inspect of the invention this object is solved by a protein comprising an amino acid sequence according to SEQ ID and NO: 1, wherein the amino acid in position 4 of SEQ ID NO: 1 is selected from the group consisting of N, S, R, T and I, preferably consisting of N, S and R and wherein the amino acid in position 5 of SEQ ID NO: 1 is selected from the group consisting of R, V, L, F, M, I, H, S, T, A, P and G, with the proviso that the amino acid sequence is not SEQ ID NO: 24.

Surprisingly, as result of the inventors' research there is an amino acid sequence motif according to SEQ ID NO: 1 that is associated with the occurrence of inflammatory diseases, especially involved in the occurrence of IBD and more prevalently involved in Crohn's disease (CD).

As used herein a “protein” refers to a polypeptide with at least 60, preferably at least 80 and more preferably at least 100 amino acids.

Preferred is a protein according to the invention, wherein the amino acid in position 7 of SEQ ID NO: 1 is A or S, wherein A is preferred.

Preferably the protein according to the invention is a protein, wherein the amino acid sequence according to SEQ ID NO: 1 is selected from the group consisting of amino acid sequences SEQ ID NO: 2 to 295.

More preferably the protein according to the invention is a TCR.

As used herein, a “T-cell receptor”, or “TCR”, is a molecule found on the surface of T-cells, or T-lymphocytes, that is responsible for recognizing antigenic molecules, more preferably peptides or/and lipids, bound to mayor histocompatibility complex (MHC) or MHC-like molecules. The TCR is a heterodimer. There are two types of TCRs: αβTCR consisting of an alpha (α) chain and a beta (β) chain (˜95% of TCRs in humans) and γδTCR consisting of a gamma and delta chain (˜5% of TCRs in humans). When the TCR engages with the antigen and MHC complex, the T-lymphocyte is activated through signal transduction, mediated by associated enzymes, co-receptors, specialized adaptor molecules, and activated or inactivated or re-leased transcription factors.

The TCRα and β chains are both composed of a variable part and a constant part. The diversity of the TCR is based mainly on genetic recombination of the DNA encoded segments, originating from the VDJ genes, in individual T cells. The process of recombination ultimately results in novel TCR amino acids sequences that allow for the recognition of antigens.

As used herein the “T-cell receptor”, and “TCR” relates both to naturally occurring T-cell receptors and recombinantly expressed T-cell receptors.

As used herein a T-cell, or a lymphocyte, is a type of lymphocytes (a subtype of white blood cells) that plays a central role in cell mediated immunity. T-cells can be distinguished from other lymphocytes, such as B-cells and natural killer cells, by the presence of a T-cell receptor on the cell surface. They are called T-cells because they mature in the thymus from thymocytes.

A protein of the invention is preferred wherein the amino acid sequence according to SEQ ID NO: 1 is comprised in the alpha chain of the TCR, more preferably in the CDR3 region.

As used herein the CDR3 region is the most variable region of a TCR. The CDR3 region of the TCR alpha chain is positioned at the junction between the V and the J TCR genes and is the result of the VDJ recombination process during T cell maturation in the thymus. The CDR3 region is the most studied region of the TCR, because has the highest contacts with the antigen.

According to the invention it is preferred that the amino acid sequence according to SEQ ID NO: 1 (or preferred sequence according to the invention) is encoded by the genes TRAV12-1 and/or TRAJ6.

Surprisingly the inventors could show that proteins according to the invention, especially the preferred proteins according to the invention were found in (human) patients affected by an inflammatory disease, especially an inflammatory bowel disease (IBD) and more especially Crohn's disease in a significant higher number as compared to healthy control or non-inflammatory disease control individuals. In addition, the cumulative abundance of the proteins according to the invention was also significantly higher in CD patients' blood.

Further, the presence and abundance of proteins according to the invention did not seem to correlate with any specific MHC-allele. Nor was found a correlation between the abundance of those proteins and other clinical parameters such as age, gender, disease severity or medication history.

In a second aspect of the invention the above object is solved by a T cell, comprising the protein of the invention. Preferably such a T cell is a human T cell.

More preferably the T cell according to the invention is a CD4⁺ cell and/or a CD8⁺ cell and/or a CD161+ cell, preferably a CD8⁺ cell.

CD4, CD8 and CD161 are proteins expressed on the surface of T lymphocytes or/and other immune cells. These proteins work as co-stimulatory molecules in different cellular signals, including but not limited to the TCR response.

A “CD4⁺ cell”, as used herein, is a cell that expresses the CD4 protein on its surface or expresses the mRNA encoding for the CD4 protein.

A “CD8⁺ cell”, as used herein, a cell that expresses the CD8 protein on its surface or expresses the mRNA encoding for the CD8 protein.

A “CD161⁺ cell”, as used herein, is a cell that expresses the CD161 protein on its surface or expresses the mRNA encoding for the CD161 protein.

The T cells according to the invention are a good basis for further research for medicaments and antibodies to be used in the treatment inflammatory diseases especially in the treatment of IBD and more especially in the treatment of CD.

In a third aspect of the invention the above object is solved by a nucleotide encoding for an amino acid sequence according to sequence ID NO: 1, preferably according to any of amino acid sequences SEQ ID NO: 2 to 295, wherein the nucleotide sequence more preferably is selected from the DNA sequences SEQ ID NO: 296 to 1205, with the proviso that DNA sequences SEQ ID NO: 638, 647, 655, 662, 721, 1020, 1098, 1130, 1137, 1147 and 1150 are excluded or an mRNA sequence encoded by a DNA sequence according to any of DNA sequence SEQ ID NO: 296 to 1205 with the proviso that DNA sequences SEQ ID NO: 364, 568, 580, 637, 638, 647, 655, 662, 721, 1020, 1098, 1130, 1137, 1147 and 1150 are excluded.

Preferably, a nucleotide sequence according to the invention is an mRNA sequence.

On basis of the information comprised in the other specification the persons skilled in the art easily recognises that the proteins according to the invention, that the T cells according to the invention and that the nucleotide sequences according to the invention can be used as markers for inflammatory bowel disease (IBD) and more especially for Crohn's disease. This includes also a protein comprising an amino acid sequence according to SEQ ID NO: 24 and a T cell comprising such an amino acid as well as a DNA or mRNA according to the invention and preferably also a DNA comprising the DNA sequences SEQ ID NO: 364, 568, 580, 637, 638, 647, 655, 662, 721, 1020, 1098, 1130, 1137, 1147 and 1150 or a mRNA comprising a sequences encoded by any of those DNA sequences.

It has further noted that those markers could be used the identification of inflammatory bowel disease in the patient and preferably for discrimination Crohn's disease in a patient from IBD other than Crohn's disease.

A fourth aspect of the present invention is a use of a protein according to the invention, or of a T cell according to the invention or of an mRNA according to the invention in the research for a medicament for treatment of a chronic inflammatory disease preferably inflammatory bowel disease (IBD), with the proviso that amino acid sequence SEQ ID NO: 24 is not excluded, and DNA sequences SEQ ID NO: 364, 568, 580, 637, 638, 647, 655, 662, 721, 1020, 1098, 1130, 1137, 1147 and 1150 are not excluded.

More preferred is a use according to the invention, wherein the chronic inflammatory disease is IBD, preferably Crohn's disease (CD) or ulcerative colitis (UC), preferably CD, more preferably ileal CD and/or ileocolonic CD.

A fifth aspect according to the invention is a use of a protein according to the invention or a use of a T cell according to the invention or a use of an mRNA according to the invention in the development of an antibody directed against the protein and/or the T cell or the mRNA.

A preferred use according to the invention is a use wherein the antibody is directed against an epitope, comprising at least a part of the amino acid sequence according to SEQ ID NO: 1, wherein preferably the amino acid sequence according to SEQ ID NO: 1 is further defined as described above.

A sixth aspect of the invention is an antibody directed against an epitope, comprising at least a part of the amino acid sequence according to SEQ ID NO: 1, wherein preferably the amino acid sequence according to SEQ ID NO: 1 is further defined as above described and/or wherein the antibody is a monoclonal antibody.

A “monoclonal antibody” as used herein comprises antibodies with the same monovalent affinity, thus binding to the same epitope.

Certainly, in the sense of the present invention the antibodies according to the invention are preferably specific antibodies wherein “specific” means the antibody is able to bind the target epitope according to the invention and/or the antibody can discriminate between that epitope and another similar or dissimilar epitope.

A further aspect of the invention is the method of discriminating blood samples of IBD patients from blood samples of individuals without IBD, preferably a method of discriminating blood samples of Crohn's disease patients from blood samples of individuals without IBD and individuals with IBD other than Crohn's disease comprising the step of quantitatively assessing the abundance of proteins according to the invention and/or the abundance of T-cells according to the invention.

As shown in the examples, it is possible to identify IBD patients form blood samples and even preferably to discriminate Crohn's disease from other IBD diseases.

In view of the above, the person's skilled in the art also understands that the proteins according to the invention, the T cells according to the invention and the nucleotide sequence according to the invention could be helpful in prognosis and/or diagnosis of IBD and preferably of Crohn's disease.

### EXAMPLES

### Material and Methods

### Example 1

### Whole Blood Sample Collection

Phenotypic details for this collection are described in Table 1.

CD patient collection: BioCrohn is a non-interventional prospective registry of Crohn's disease patients undergoing biologics treatment which was established by the German “Competence Network IBD”, Kiel, Germany in 2008. Samples included in this study were collected prior to the start of anti-TNF therapy (Infliximab).

To evaluate disease activity, the Harvey-Bradshaw Index (HBI) was used. This measure was employed to distinguish between inactive (0≤HBI≤4: remission) and active state of the disease (5≤HBI≤7: mild activity, 8≤HBI≤16: moderate activity, 17≤HBI≤∞: severe activity) (42), as described from the Canadian society of intestinal research. In total, whole blood samples of 109 CD patients were available for analysis. All patients had active disease at sampling time. Response to anti-TNF therapy was defined as a steroid-independent change from active disease (HBI>4 at baseline) to an inactive state of the disease (HBI<4 at 6 months after start of therapy). Accordingly, non-response to anti-TNF therapy was defined as the disease being consistently active at both time points under investigation (HBI>4 at baseline and 6 months).

UC patient collection: blood samples from UC patients of the “whole blood collection” were recruited as part of a larger panel of patients with IBD, recruited in the Comprehensive Centre for Inflammation Medicine (CCIM), at the University Hospital Schleswig-Holstein, Campus Kiel (Germany). Samples for the present study were randomly selected among the available samples stored in the PopGen biobank. In total, whole blood samples of 36 UC patients were available.

Healthy control collection: blood samples were obtained from the PopGen control sample, consisting of a community-based sample from the Kiel area (Northern Germany) and of blood donors from the University Hospital Schleswig-Holstein, Campus Kiel(43). In total, 1316 individuals were recruited at baseline (between 2005 and 2007) and were re-examined at the second examination cycle; between 2010 and 2012; n=952)(44, 45). Participants filled-in questionnaires on health and nutrition, received a basic clinical examination and provided blood samples for research. For the present analyses, individuals with major diseases such as cancer, neurological and inflammatory diseases were excluded. In total, 99 whole blood samples were available. These samples were matched to the CD patients in regard to age and sex.

The study was approved by the local ethics committees (Ethics votes: A156/03 v. 29.7.2010, A156/03-3/15 v. 21.1.16, A156/03-2/13, A161/08). All study participants gave their written informed consent.

2.5 ml of blood were drawn and stored in PAXgene tubes (Qiagen), following the manufacturer's instructions. Samples were stored at −80° C. Total RNA was isolated using the PAXgene blood miRNA kit (Qiagen) with automatic isolation using the Qiacube machine. 1,200 ng of total RNA were used for

TABLE 1
Whole blood collection phenotype information. Information about disease phenotype was provided
via the Montreal classification. Information on the disease activity was available through
the Harvey-Bradshaw index (HBI) for CD patients and though the colitis activity index
(CAI) and partial Mayo score for UC patients. Information about steroid therapy at sampling
time was also provided. “NA” corresponds to “information not available”.

| | Sample group | | |
|---|---|---|---|
| | CD | UC | Healthy |
| Basic phenotype | | | |
| Samples | 109 | 36 | 99 |
| Gender, Male/Female (%) | 43/65 (40/60%) | 21/15 (58/42%) | 41/58 (41/59%) |
| Age, mean ± std | 40 ± 12 | 37 ± 13 | 48 ± 13 |
| Montreal classification | | | |
| Age at diagnosis in years | | | |
| A1, <16) (%) | 7 (6%) | — | — |
| A2, 17-40 (%) | 74 (70%) | — | — |
| A3, >40 (%) | 25 (24%) | — | — |
| Behaviour | | | |
| B1, inflammatory (%) | 51 (47%) | — | — |
| B2, stricturing (%) | 21 (19%) | — | — |
| B3, penetrating (%) | 36 (34%) | — | — |
| Location | | | |
| L1, ileal (%) | 29 (27%) | — | — |
| L2, colonic (%) | 23 (21%) | — | — |
| L3, ileocolonic (%) | 56 (52%) | — | — |
| Disease extent | | | |
| E1, proctitis (%) | — | 3 (3%) | — |
| E2, left-sided UC (%) | — | 16 (47%) | — |
| E3, extensive UC (%) | — | 15 (44%) | — |
| Info not available (NA) | 1 | 2 | — |
| Disease activity | | | |
| HBI, mean ± std | 11 ± 5 | — | — |
| Mild activity, 5 ≤ HBI ≤ 7 (%) | 37 (34%) | — | — |
| Moderate activity, 8 ≤ HBI ≤ 16 (%) | 57 (53%) | — | — |
| Severe activity, 17 ≤ HBI ≤ ∞ (%) | 14 (13%) | — | — |
| Partial Mayo score, mean ± std | — | 3.3 ± 2 | — |
| CAI, mean ± std | — | 6.7 ± 4.3 | — |
| Info not available (NA) | 1 | 0 | — |
| Medications | | | |
| Steroids, yes/no (%) | 7/102 (6/94%) | 14/22 (39/61%) | — |
| Treatment naïve yes/no/NA (%) | 77/14/18 (70/13/17%) | 0/0/36 (0/0/100%) | — |
| Responders to anti-TNF yes/no/NA (%) | 46/60/3 (42/55/3%) | — | — |

### Example 2

### Surgery Sample Collection

Samples were collected in collaboration with the Clinic for General and Thoracic Surgery of the University Hospital Schleswig-Holstein, Kiel, Germany. It is important to notice that for CRC tissue samples, macroscopically normal, non-tumour tissue was utilized. Sample details are provided in Table 2.

Sample collection included (1) one 9 ml whole blood EDTA tube from which PBMCs were isolated by density gradient centrifugation (Ficoll, GE Healthcare) and (2) resected intestinal mucosa. Tissues were cut into pieces of approximately 30-50 mg, snap-frozen and stored at −80° C. Details on the number of collected samples and intestinal resection location for each patient are available in Table 2, together with patients' basic phenotypic information.

Total RNA was isolated using the RNeasy mini kit from Qiagen following the manufacturer's instructions. Up to 3,200 ng of total RNA were used for TCR library preparation. Details on the sorted T cell populations from additional surgery samples are described in the supplementary materials and methods as well as supplementary table 1.

TABLE 2
Surgery collection phenotype information. Information about the numbers
of collected samples for each tissue is provided together with information
about intestinal location of the sample and about therapy with steroids
or immunosuppressants at time of surgery.

| | CD | UC | CRC |
|---|---|---|---|
| Basic phenotype | | | |
| Samples | 11 | 13 | 13 |
| Gender, Male/Female (%) | 4/7 | 7/6 | 8/5 |
| | (36/64%) | (54/46%) | (62/38%) |
| Age, mean ± std | 40 ± 15 | 52 ± 16 | 69 ± 12 |
| Sample types | | | |
| Intestinal samples | 11 | 13 | 9 |
| Mesenteric lymph nodes | 4 | 5 | 0 |
| PBMCs | 11 | 12 | 12 |
| Intestine + PBMCs datasets | 11 | 12 | 8 |
| Resected tissue location | | | |
| Large intestine | 4 | 13 | 13 |
| Small intestine | 7 | 0 | 0 |
| Medications | | | |
| Steroids, yes/no/NA | 4/5/2 | 7/6/0 | 0/0/13 |
| Immunosuppressants, yes/no/NA | 5/5/1 | 2/10/1 | 0/0/13 |

### Example 3

### TCR Library Preparation and Sequencing

Starting from total RNA, molecular-barcoded TCR cDNA libraries were prepared as previously described in M. V. Pogorelyy et al., Persisting fetal clonotypes influence the structure and overlap of adult human T cell receptor repertoires. PLOS Computational Biology 13, e1005572-e1005572 (2017), with minor modifications for both TCRα and TCRβ chains. Briefly, cDNA synthesis was performed using SMARTScribe reverse transcriptase (Clontech, Takara) using primers for the TCRα and TCRβ constant region. A unique molecular identifier (UMI), and a sample barcode of 6 nucleotides, were introduced via template-switching. cDNA synthesis was carried out for 60 minutes at 42° C. cDNA was then treated with Uracil DNA-Glycosylase (UDG, from New England Biolabs) and incubated for 30 min at 37° C. Samples were subsequently purified with the QIAquick PCR purification kit (Qiagen) and eluted in 50 or 100 μl deionized water. Purified cDNA was then amplified with 2 consecutive PCRs, respectively 18 and 12 cycles, with purification after each PCR using MagSi-NGSprep Plus (MagnaMedics). Illumina compatible adapters and sample-specific barcodes were added during the second PCR. Quality and concentration of the libraries were measured with TapeStation D1000 (Agilent) and Qubit (Thermofisher). Libraries were pooled using 5 ng per library and sequenced on Illumina HiSeq2500 with a single-index Rapid Run of 2×100 bp (whole blood) or on Illumina NovaSeq 6000 SP 2×150 bp flow cell (surgery). Custom sequencing primers were added to the Illumina primers.

For the sorted surgery collection samples, libraries were prepared using human TCR profiling kit (MiLaboratory), according to the manufacturers' protocol. Libraries were sequenced on Illumina MiSeq 2×150 bp or NovaSeq 6000 SP 2×150 bp.

### Example 4

### TCR Data Pre-Processing

PCR and sequencing error correction were performed through identification and selection of unique molecular identifiers using the software MiGEC, version 1.2.6. Filtered sequences were aligned on a TCR gene reference, clonotypes were identified, grouped and CDR3 sequence was identified using the software MiXCR, version 2.1.1 (first sample collection: blood collection) or version 3.0.14 (second collection: surgery collection). Clonotype tables containing clonotype counts, frequencies, CDR3 nucleotide and amino acid sequences and V(D)J genes were obtained and used for further analysis.

A total of 444 samples were analysed for bulk TCR repertoire.

### Example 5

### TCR Data Analysis

For statistical analysis, the Wilcoxon-Mann-Whitney U test was used unless otherwise indicated. Samples with less than 200 identified unique clonotypes were excluded from the analysis. TCRs found at a count inferior to 2 (singletons) or encoding for out of frame sequences or TCRs containing stop codons, were also excluded from further analyses.

Proportion analysis of TCR groups (CDR3length_Vgene_Jgene): For each disease group (CD, UC, healthy), the total number of TCR sequences being part of each TCR group (CDR3length_Vgene_Jgene) was calculated and divided by the number of total TCR sequences present in each disease group in order to normalize for differences in TCR number among disease groups. The proportion of TCRs of each TCR group was then compared between disease groups, either CD against UC, or against healthy controls, for both TCR alpha and TCR beta.

Fisher test on identified TCR groups of interest: using specific CDR3 and VJ gene combinations identified through the analysis of repertoire proportions (12aa_TRAV1-2_TRAJ33, 15aa_TRAV12-1_TRAJ6), we performed one-sided Fisher's exact test on the single TCRs of these groups to identify TCR alpha clonotypes respectively decreased (12aa_TRAV1-2_TRAJ33) or increased (15aa_TRAV12-1_TRAJ6) in CD patients versus healthy individuals, from the whole blood and surgery collections. We applied multiple testing correction through the Benjamini-Hochberg (BH) method(49), in order to control expected proportion of false discoveries amongst the rejected hypotheses (false discovery rate, FDR).

Analysis on specific sequences: TCR sequences from the identified group of CDR3length=15 amino acids, V gene=TRAV12-1 and J gene=TRAJ6 that were found to be more abundant in CD patients as compared to controls in the whole blood sample collection were used for the logo plot which defined the specific clonotype motif CVV**A*GGSYIPTF (SEQ ID NO: 1). All sequences of the selected TCR group, carrying the motif were selected for further analysis. For each sample, the number of sequences according to the invention present in the sample as well as the sum of relative abundances (cumulative abundance) of CAIT sequences according to the invention were calculated.

The results can be found in the table 3 (amino acid sequences) and in the sequence protocol (SEQ ID NO: 2-295 (amino acid sequences) and SEQ ID NO: 296-1205 (DNA sequences)).

As can be seen from table 3 there are polypeptide sequences with higher occurrences. It has to been noted that according to the invention a polypeptide sequence is the more preferred the higher the occurrence is according to table 3.

TABLE 3
TCR sequences

| SEQ ID NO | Occurrencies | Sequence |
|---|---|---|
| 2 | 144 | CVVNLASGGSYIPTF |
| 3 | 93 | CVVNGASGGSYIPTF |
| 4 | 88 | CVVNSASGGSYIPTF |
| 5 | 77 | CVVNIASGGSYIPTF |
| 6 | 77 | CVVNPASGGSYIPTF |
| 7 | 75 | CVVNRASGGSYIPTF |
| 8 | 72 | CVVSLASGGSYIPTF |
| 9 | 62 | CVVNMASGGSYIPTF |
| 10 | 55 | CVVTLASGGSYIPTF |
| 11 | 49 | CVVNTASGGSYIPTF |
| 12 | 43 | CVVRLASGGSYIPTF |
| 13 | 42 | CVVRHASGGSYIPTF |
| 14 | 38 | CVVNVASGGSYIPTF |
| 15 | 37 | CVVSHASGGSYIPTF |
| 16 | 36 | CVVRVASGGSYIPTF |
| 17 | 34 | CVVRSASGGSYIPTF |
| 18 | 33 | CVVIPASGGSYIPTF |
| 19 | 31 | CVVNKASGGSYIPTF |
| 20 | 30 | CVVRGASGGSYIPTF |
| 21 | 30 | CVVTPASGGSYIPTF |
| 22 | 29 | CVVNAASGGSYIPTF |
| 23 | 29 | CVVNFASGGSYIPTF |
| 24 | 24 | CVVSSASGGSYIPTF |
| 25 | 23 | CVVNQASGGSYIPTF |
| 26 | 21 | CVVRTASGGSYIPTF |
| 27 | 19 | CVVIHASGGSYIPTF |
| 28 | 19 | CVVNEASGGSYIPTF |
| 29 | 19 | CVVNHASGGSYIPTF |
| 30 | 19 | CVVRRASGGSYIPTF |
| 31 | 18 | CVVILASGGSYIPTF |
| 32 | 18 | CVVRIASGGSYIPTF |
| 33 | 16 | CVVRAASGGSYIPTF |
| 34 | 16 | CVVSPASGGSYIPTF |
| 35 | 16 | CVVSTASGGSYIPTF |
| 36 | 16 | CVVTHASGGSYIPTF |
| 37 | 16 | CVVTSASGGSYIPTF |
| 38 | 16 | CVVTVASGGSYIPTF |
| 39 | 15 | CVVNMAAGGSYIPTF |
| 40 | 15 | CVVSVASGGSYIPTF |
| 41 | 15 | CVVTAASGGSYIPTF |
| 42 | 14 | CVVKGASGGSYIPTF |
| 43 | 12 | CVVNGAPGGSYIPTF |
| 44 | 12 | CVVNIAAGGSYIPTF |
| 45 | 12 | CVVNMATGGSYIPTF |
| 46 | 12 | CVVPHASGGSYIPTF |
| 47 | 12 | CVVSAASGGSYIPTF |
| 48 | 12 | CVVTFASGGSYIPTF |
| 49 | 11 | CVVREASGGSYIPTF |
| 50 | 11 | CVVSFASGGSYIPTF |
| 51 | 11 | CVVSGASGGSYIPTF |
| 52 | 11 | CVVSIASGGSYIPTF |
| 53 | 10 | CVVIAASGGSYIPTF |
| 54 | 10 | CVVISASGGSYIPTF |
| 55 | 10 | CVVNMAGGGSYIPTF |
| 56 | 9 | CVVFHASGGSYIPTF |
| 57 | 9 | CVVRQASGGSYIPTF |
| 58 | 9 | CVVSRASGGSYIPTF |
| 59 | 8 | CVVIGASGGSYIPTF |
| 60 | 8 | CVVLHASGGSYIPTF |
| 61 | 8 | CVVNIATGGSYIPTF |
| 62 | 8 | CVVNYASGGSYIPTF |
| 63 | 8 | CVVRFASGGSYIPTF |
| 64 | 8 | CVVTLAGGGSYIPTF |
| 65 | 7 | CVVAHASGGSYIPTF |
| 66 | 7 | CVVIRASGGSYIPTF |
| 67 | 7 | CVVNGAAGGSYIPTF |
| 68 | 7 | CVVNIAGGGSYIPTF |
| 69 | 7 | CVVRMASGGSYIPTF |
| 70 | 7 | CVVTIASGGSYIPTF |
| 71 | 6 | CVVKHASGGSYIPTF |
| 72 | 6 | CVVKSASGGSYIPTF |
| 73 | 6 | CVVMGASGGSYIPTF |
| 74 | 6 | CVVNDASGGSYIPTF |
| 75 | 6 | CVVNRAPGGSYIPTF |
| 76 | 6 | CVVTGASGGSYIPTF |
| 77 | 5 | CVVALASGGSYIPTF |
| 78 | 5 | CVVDHASGGSYIPTF |
| 79 | 5 | CVVIFASGGSYIPTF |
| 80 | 5 | CVVIIASGGSYIPTF |
| 81 | 5 | CVVIVASGGSYIPTF |
| 82 | 5 | CVVKPASGGSYIPTF |
| 83 | 5 | CVVKRASGGSYIPTF |
| 84 | 5 | CVVKTASGGSYIPTF |
| 85 | 5 | CVVNLAAGGSYIPTF |
| 86 | 5 | CVVNSAAGGSYIPTF |
| 87 | 5 | CVVNSAGGGSYIPTF |
| 88 | 5 | CVVRKASGGSYIPTF |
| 89 | 5 | CVVSQASGGSYIPTF |
| 90 | 4 | CVVFLASGGSYIPTF |
| 91 | 4 | CVVFSASGGSYIPTF |
| 92 | 4 | CVVKAASGGSYIPTF |
| 93 | 4 | CVVKLASGGSYIPTF |
| 94 | 4 | CVVMHASGGSYIPTF |
| 95 | 4 | CVVNLAGGGSYIPTF |
| 96 | 4 | CVVNMAPGGSYIPTF |
| 97 | 4 | CVVNRAAGGSYIPTF |
| 98 | 4 | CVVNRATGGSYIPTF |
| 99 | 4 | CVVNVAAGGSYIPTF |
| 100 | 4 | CVVNVATGGSYIPTF |
| 101 | 4 | CVVNWASGGSYIPTF |
| 102 | 4 | CVVSKASGGSYIPTF |
| 103 | 4 | CVVSQAAGGSYIPTF |
| 104 | 4 | CVVTDASGGSYIPTF |
| 105 | 4 | CVVTYASGGSYIPTF |
| 106 | 3 | CVVGLASGGSYIPTF |
| 107 | 3 | CVVGTASGGSYIPTF |
| 108 | 3 | CVVIDASGGSYIPTF |
| 109 | 3 | CVVITASGGSYIPTF |
| 110 | 3 | CVVKVASGGSYIPTF |
| 111 | 3 | CVVLLASGGSYIPTF |
| 112 | 3 | CVVNGATGGSYIPTF |
| 113 | 3 | CVVNIAPGGSYIPTF |
| 114 | 3 | CVVNKAAGGSYIPTF |
| 115 | 3 | CVVNLARGGSYIPTF |
| 116 | 3 | CVVNSAVGGSYIPTF |
| 117 | 3 | CVVNTAGGGSYIPTF |
| 118 | 3 | CVVNVAGGGSYIPTF |
| 119 | 3 | CVVTQASGGSYIPTF |
| 120 | 3 | CVVTTASGGSYIPTF |
| 121 | 3 | CVVYHASGGSYIPTF |
| 122 | 3 | CVVYLASGGSYIPTF |
| 123 | 2 | CVVADASGGSYIPTF |
| 124 | 2 | CVVAQASGGSYIPTF |
| 125 | 2 | CVVASASGGSYIPTF |
| 126 | 2 | CVVATASGGSYIPTF |
| 127 | 2 | CVVFGASGGSYIPTF |
| 128 | 2 | CVVHHASGGSYIPTF |
| 129 | 2 | CVVHVASGGSYIPTF |
| 130 | 2 | CVVINASGGSYIPTF |
| 131 | 2 | CVVIQASGGSYIPTF |
| 132 | 2 | CVVKEASGGSYIPTF |
| 133 | 2 | CVVKIASGGSYIPTF |
| 134 | 2 | CVVKKASGGSYIPTF |
| 135 | 2 | CVVKMASGGSYIPTF |
| 136 | 2 | CVVKRAGGGSYIPTF |
| 137 | 2 | CVVLGASGGSYIPTF |
| 138 | 2 | CVVLPASGGSYIPTF |
| 139 | 2 | CVVLSASGGSYIPTF |
| 140 | 2 | CVVLTASGGSYIPTF |
| 141 | 2 | CVVMPASGGSYIPTF |
| 142 | 2 | CVVNEAAGGSYIPTF |
| 143 | 2 | CVVNIALGGSYIPTF |
| 144 | 2 | CVVNIARGGSYIPTF |
| 145 | 2 | CVVNMAEGGSYIPTF |
| 146 | 2 | CVVNPATGGSYIPTF |
| 147 | 2 | CVVNSATGGSYIPTF |
| 148 | 2 | CVVPFASGGSYIPTF |
| 149 | 2 | CVVPPASGGSYIPTF |
| 150 | 2 | CVVPRASGGSYIPTF |
| 151 | 2 | CVVPSASGGSYIPTF |
| 152 | 2 | CVVPTASGGSYIPTF |
| 153 | 2 | CVVRLAGGGSYIPTF |
| 154 | 2 | CVVRPASGGSYIPTF |
| 155 | 2 | CVVRTARGGSYIPTF |
| 156 | 2 | CVVRVAAGGSYIPTF |
| 157 | 2 | CVVRYASGGSYIPTF |
| 158 | 2 | CVVSDASGGSYIPTF |
| 159 | 2 | CVVSGAVGGSYIPTF |
| 160 | 2 | CVVSLAGGGSYIPTF |
| 161 | 2 | CVVVHASGGSYIPTF |
| 162 | 2 | CVVVLASGGSYIPTF |
| 163 | 1 | CVVAFASGGSYIPTF |
| 164 | 1 | CVVAGASGGSYIPTF |
| 165 | 1 | CVVAPASGGSYIPTF |
| 166 | 1 | CVVAVASGGSYIPTF |
| 167 | 1 | CVVAWASGGSYIPTF |
| 168 | 1 | CVVDDASGGSYIPTF |
| 169 | 1 | CVVDQASGGSYIPTF |
| 170 | 1 | CVVEQASGGSYIPTF |
| 171 | 1 | CVVFAASGGSYIPTF |
| 172 | 1 | CVVFLAGGGSYIPTF |
| 173 | 1 | CVVFQARGGSYIPTF |
| 174 | 1 | CVVFQASGGSYIPTF |
| 175 | 1 | CVVFRASGGSYIPTF |
| 176 | 1 | CVVFTASGGSYIPTF |
| 177 | 1 | CVVFVASGGSYIPTF |
| 178 | 1 | CVVGDASGGSYIPTF |
| 179 | 1 | CVVGIASGGSYIPTF |
| 180 | 1 | CVVGRASGGSYIPTF |
| 181 | 1 | CVVGVAAGGSYIPTF |
| 182 | 1 | CVVGVASGGSYIPTF |
| 183 | 1 | CVVHDASGGSYIPTF |
| 184 | 1 | CVVHEASGGSYIPTF |
| 185 | 1 | CVVHLASGGSYIPTF |
| 186 | 1 | CVVHQASGGSYIPTF |
| 187 | 1 | CVHYASGGSYIPTF |
| 188 | 1 | CVVIPATGGSYIPTF |
| 189 | 1 | CVVISAPGGSYIPTF |
| 190 | 1 | CVVIYASGGSYIPTF |
| 191 | 1 | CVVKAAAGGSYIPTF |
| 192 | 1 | CVVKDASGGSYIPTF |
| 193 | 1 | CVVKFASGGSYIPTF |
| 194 | 1 | CVVKGAGGGSYIPTF |
| 195 | 1 | CVVKGAPGGSYIPTF |
| 196 | 1 | CVVKLAVGGSYIPTF |
| 197 | 1 | CVVKNASGGSYIPTF |
| 198 | 1 | CVVKSAGGGSYIPTF |
| 199 | 1 | CVVKTAAGGSYIPTF |
| 200 | 1 | CVVKTAGGGSYIPTF |
| 201 | 1 | CVVKVAGGGSYIPTF |
| 202 | 1 | CVVKYASGGSYIPTF |
| 203 | 1 | CVVLGAAGGSYIPTF |
| 204 | 1 | CVVLVASGGSYIPTF |
| 205 | 1 | CVVMAASGGSYIPTF |
| 206 | 1 | CVVMKAEGGSYIPTF |
| 207 | 1 | CVVMLASGGSYIPTF |
| 208 | 1 | CVVMRASGGSYIPTF |
| 209 | 1 | CVVMTASGGSYIPTF |
| 210 | 1 | CVVNAAAGGSYIPTF |
| 211 | 1 | CVVNAAPGGSYIPTF |
| 212 | 1 | CVVNAARGGSYIPTF |
| 213 | 1 | CVVNAATGGSYIPTF |
| 214 | 1 | CVVNEAGGGSYIPTF |
| 215 | 1 | CVVNEATGGSYIPTF |
| 216 | 1 | CVVNGAGGGSYIPTF |
| 217 | 1 | CVVNGAQGGSYIPTF |
| 218 | 1 | CVVNGARGGSYIPTF |
| 219 | 1 | CVVNGAVGGSYIPTF |
| 220 | 1 | CVVNIAEGGSYIPTF |
| 221 | 1 | CVVNIAIGGSYIPTF |
| 222 | 1 | CVVNKAKGGSYIPTF |
| 223 | 1 | CVVNKAPGGSYIPTF |
| 224 | 1 | CVVNKATGGSYIPTF |
| 225 | 1 | CVVNLAVGGSYIPTF |
| 226 | 1 | CVVNMALGGSYIPTF |
| 227 | 1 | CVVNMAQGGSYIPTF |
| 228 | 1 | CVVNMARGGSYIPTF |
| 229 | 1 | CVVNNAPGGSYIPTF |
| 230 | 1 | CVVNNARGGSYIPTF |
| 231 | 1 | CVVNNASGGSYIPTF |
| 232 | 1 | CVVNPALGGSYIPTF |
| 233 | 1 | CVVNPAPGGSYIPTF |
| 234 | 1 | CVVNQAAGGSYIPTF |
| 235 | 1 | CVVNQATGGSYIPTF |
| 236 | 1 | CVVNRAGGGSYIPTF |
| 237 | 1 | CVVNSAEGGSYIPTF |
| 238 | 1 | CVVNSAKGGSYIPTF |
| 239 | 1 | CVVNTAAGGSYIPTF |
| 240 | 1 | CVVNTAKGGSYIPTF |
| 241 | 1 | CVVNTAPGGSYIPTF |
| 242 | 1 | CVVNTARGGSYIPTF |
| 243 | 1 | CVVNVAPGGSYIPTF |
| 244 | 1 | CVVNWAAGGSYIPTF |
| 245 | 1 | CVVNWAGGGSYIPTF |
| 246 | 1 | CVVPEASGGSYIPTF |
| 247 | 1 | CVVPGASGGSYIPTF |
| 248 | 1 | CVVPLASGGSYIPTF |
| 249 | 1 | CVVPLATGGSYIPTF |
| 250 | 1 | CVVPPAPGGSYIPTF |
| 251 | 1 | CVVPVASGGSYIPTF |
| 252 | 1 | CVVQGASGGSYIPTF |
| 253 | 1 | CVVQHASGGSYIPTF |
| 254 | 1 | CVVQLASGGSYIPTF |
| 255 | 1 | CVVQSASGGSYIPTF |
| 256 | 1 | CVVRDASGGSYIPTF |
| 257 | 1 | CVVREAWGGSYIPTF |
| 258 | 1 | CVVRFARGGSYIPTF |
| 259 | 1 | CVVRGAEGGSYIPTF |
| 260 | 1 | CVVRHATGGSYIPTF |
| 261 | 1 | CVVRQAGGGSYIPTF |
| 262 | 1 | CVVRRAAGGSYIPTF |
| 263 | 1 | CVVRSATGGSYIPTF |
| 264 | 1 | CVVRTAQGGSYIPTF |
| 265 | 1 | CWVRVAGGGSYIPTF |
| 266 | 1 | CVVSEASGGSYIPTF |
| 267 | 1 | CVVSGAQGGSYIPTF |
| 268 | 1 | CVVSLAAGGSYIPTF |
| 269 | 1 | CVVSLAKGGSYIPTF |
| 270 | 1 | CVVSMASGGSYIPTF |
| 271 | 1 | CVVSPAPGGSYIPTF |
| 272 | 1 | CVVSQAGGGSYIPTF |
| 273 | 1 | CVVSRAMGGSYIPTF |
| 274 | 1 | CVVSSAVGGSYIPTF |
| 275 | 1 | CVVSTATGGSYIPTF |
| 276 | 1 | CVVTEASGGSYIPTF |
| 277 | 1 | CVVTGAAGGSYIPTF |
| 278 | 1 | CVVTIAEGGSYIPTF |
| 279 | 1 | CVVTNAPGGSYIPTF |
| 280 | 1 | CVVTPAPGGSYIPTF |
| 281 | 1 | CVVTRASGGSYIPTF |
| 282 | 1 | CVVTSARGGSYIPTF |
| 283 | 1 | CVVTSATGGSYIPTF |
| 284 | 1 | CVVTTAGGGSYIPTF |
| 285 | 1 | CVVVFASGGSYIPTF |
| 286 | 1 | CVVVGASGGSYIPTF |
| 287 | 1 | CVVVMASGGSYIPTF |
| 288 | 1 | CVVVRASGGSYIPTF |
| 289 | 1 | CVVWGASGGSYIPTF |
| 290 | 1 | CVVWPASGGSYIPTF |
| 291 | 1 | CVVWTASGGSYIPTF |
| 292 | 1 | CVVYFASGGSYIPTF |
| 293 | 1 | CVVYGASGGSYIPTF |
| 294 | 1 | CVVYMAPGGSYIPTF |
| 295 | 1 | CVVYPASGGSYIPTF |

### Example 6

### Single-Cell Analysis

Blood was drawn from three CD patients, 2 females and 1 male of age 29, 49 and 36 year old respectively, who were shown to have high abundance of TCRs according to the invention during bulk TCR analysis, as well as from three age and sex matched healthy controls. PBMCs were isolated by gradient centrifugation from EDTA blood tubes. CD3+ cells were enriched by MACS (Miltenyi Biotec).

For single-cell analysis, CD3+ (VioBle) CD45RO+ (PeVio770) memory T cells were sorted in CD8+ (VioGreen) and CD4+ (APCVio770) subpopulations. Cells were removed from the sorting chamber into pre-coated low-bind collection tubes and centrifuged for 5 min at 400×g, 4° C. Cells were re-suspended in PBS plus 0.04% BSA to a concentration of 1000 cells/ul.

Single-cell suspensions were loaded on a Chromium Chip G (10× Genomics) according to manufacturer's instructions for processing with the Chromium Next GEM Single Cell 5′ Library and Gel Bead Kit v1.1. Depending on the number of cells available for each patient, a maximum of 20,000 cells were loaded for each reaction. TCR single-cell libraries were subsequently prepared from the same cells with the Chromium Single Cell V(D)J Enrichment Kit, Human T Cell. Libraries were sequenced on the Illumina NovaSeq 6000 machine with 2×100 bp for gene expression, aiming for 50,000 reads per cell and 2×150 bp and 5000 reads per cell for TCR libraries.

Single-cell T cell receptor repertoire clonotype tables were generated using the VDJ command of the Cellranger software, version 3.1.0 from 10×Genomics and using the VDJ reference version 2.0.0. Clonotype tables were filtered in order to include only cells which passed quality filtering in the gene expression analysis. In addition, clonotypes were stringently filtered for possible doublets by removing clonotypes (i) found in 1 cell only and containing more than 1 TCR alpha and 1 TCR beta (ii) containing more than 1 TCR alpha and no TCR beta sequence (iii) containing more than 1 TCR beta and no TCR alpha sequence (iv) containing more than 2 TCR alpha or more than 2 TCR beta sequences.

Gene expression matrices were generated through the COUNT command of Cellranger v3.1.0. from 10×Genomics and using the reference GRCh38 version 3.0. Data were analyzed using the Seurat v3.2.3 R package (24). Cells with more than 400 but less than 3500 detected RNA features per cell and less than 8% mitochondrial RNA were retained for further analysis. Only genes present in at least 1% of the cells were considered. Also, only cells with a detected TCR were used. TCR VDJ genes were removed from gene expression counts to allow for an unbiased analysis. Data from multiple individuals were merged and batch effect correction by experimental day and sequencing run was performed using the Harmony package. 20 dimensions were used for performing principal component analysis (PCA) and uniform manifold approximation and projection (UMAP) in 3 dimensions. Clusters were identified using 0.4 resolution. Positive and negative marker genes were identified using Seurat FindMarkers function and the MAST method, considering only genes found in at least 25% of cells of each cluster.

Cells according to the invention were defined from TCR alpha of 15aa, TRAV12-1 and TRAJ6. Proportion of CAIT cells carrying certain TRBV genes was calculated on the number of unique CAIT clonotypes identified, independently from the number of cells carrying each clonotypes and originated by the same original clonotype by clonal expansion.

### Example 7

### Sample Collection and Cell Staining for Flow Cytometry Immunophenotyping

Whole blood samples were collected in 9 ml EDTA tubes.

Crohn's disease patients: samples were collected in collaboration with the Comprehensive Center for Inflammation Medicine (CCIM) of the University Hospital Schleswig-Holstein, Kiel, Germany. A total of 20 CD patients were recruited. Sample details are provided in Supplementary Table 5.

Healthy human blood: 16 samples were collected from volunteer workers at the University Hospital Schleswig-Holstein, Kiel, Germany.

Peripheral blood mononuclear cells (PBMCs) were isolated by standard density gradient (Biocoll, Biochrom, Berlin Germany) and freshly used for staining. PBMCs were washed with PBS+2 mM EDTA+0.5% BSA (PEB buffer) and then stained with a cell surface antibody cocktail for CD3 (OKT3, Biolegend), CD4 (M-T466, Miltenyi Biotec), CD8 (REA734, Miltenyi Biotec), CD14 (TÜK4, Miltenyi Biotec), CD20 (LT20, Miltenyi Biotec), CD161 (DX12, BD Biosciences), IL-18Rα/CD218a (REA1095, Miltenyi Biotec), TRAV1-2 (F1, Invitrogen), TRAV12-1 (REA179, Miltenyi Biotec), at room temperature for 30 min in the dark. Cells were washed again and resuspended in 300 uL of PEB buffer. Propidium Iodide (Miltenyi Biotec) was used to exclude dead cells. Data were acquired on a LSR Fortessa (BD Bioscience, San Jose, CA, USA). Flow cytometry data were analyzed using FlowJo (Treestar, Ashland, OR, USA) software.

TABLE 5
Immunophenotyping blood sample collection phenotype information.
Information about disease phenotype was provided via de
Montreal classification. Disease score was available through
the Harvey-Bradshaw index (HBI) for CD patients. NA corresponds
to “information not available”.

| | Sample group | |
|---|---|---|
| | CD | Healthy |
| Basic phenotype | | |
| Samples | 20 | 16 |
| Gender, M/F (%) | 11/9 (55/45%) | 7/9 (43/57%) |
| Age, mean ± std | 44 ± 18 | 31 ± 8 |
| Montreal classification | | |
| Age at diagnosis in years | | |
| A1, <16) (%) | 3 (15%) | — |
| A2, 17-40 (%) | 11 (55%) | — |
| A3, >40 (%) | 4 (20%) | — |
| Info not available (NA) | 2 | |
| Behaviour | | |
| B1, inflammatory (%) | 9 (45%) | — |
| B2, stricturing (%) | 5 (25%) | — |
| B3, penetrating (%) | 4 (20%) | — |
| Info not available (NA) | 2 | — |
| Location | | |
| L1, ileal (%) | 8 (40%) | — |
| L2, colonic (%) | 1 (12%) | — |
| L3, ileocolonic (%) | 8 (23%) | — |
| Info not available (NA) | 3 | — |
| Disease activity | | |
| HBI, mean ± std | 3 ± 3 | — |
| Remission, HBI ≤ 4 | 7 (35%) | |
| Mild activity, 5 ≤ HBI ≤ 7 (%) | 1 (5%) | — |
| Moderate activity, 8 ≤ HBI ≤ 15 (%) | 1 (5%) | — |
| Severe activity, 16 ≤ HBI ≤ ∞ (%) | — | — |
| Info not available (NA) | 11 | — |
| CRP | 6.9 ± 14 | — |

### Results

### Identification of TCRs Enriched in CD Patients' Blood

In CD patients, a group of TCR alpha chains defined by a 15 amino acids CDR3 region and with the gene combination TRAV12-1_TRAJ6 were enriched as compared to healthy controls. The Fisher's exact test confirmed that several clonotypes of the TRAV12-1_TRAJ6 group were present in more CD patients than controls. In detail, 10 clonotypes were significantly enriched after multiple comparison correction and further 3 were nominally significantly enriched. Clonotypes of this TCR group, identified as significantly enriched in CD, share the semi-invariant CDR3 motif CVV**A*GGSYIPTF (SEQ ID NO: 1).

While positions 4 and 5 of the identified motif are the most variable, positions 6 and 7 are more conserved and occupied by A and S amino acids respectively. Moreover, the predicted 3D structure of the TCR alpha CDR3 loop showed that positions 4 and 5 are mostly buried inside the TCR protein structure and may indirectly influence the loop conformation. Positions 6 and 7 are exposed and, together with positions 8 and 11, are predicted to be the most involved into epitope interaction. However, CDR3 loops are very flexible and may adapt to different conformations when interacting with different epitopes, thus, in principle, all CDR3 amino acid positions, excluding the terminal 3-4 residues, may contact the antigen and influence binding affinity.

A higher number of different clonotypes comprising the motif according to the invention (different CDR3 regions) were found in CD patients as compared to healthy controls. In addition, they were more expanded in CD patients' blood and their cumulative abundance (the sum of their relative abundance in each sample) was accordingly significantly higher in CD patients (P-value=0.008).

The inventors also investigated whether the TCRs according to the invention were associated with phenotypic or clinical traits. No significant association was detected between the cumulative abundance or the number of TCRs according to the invention, and clinical parameters such as sex, age, smoking behaviour, disease location, disease severity or previous treatment for CD. Samples from CD patients were collected prior to start of treatment with anti-TNF biologics. A trend was observed, showing increased numbers of clonotypes comprising the motif according to the invention in patients non-responding to anti-TNF therapy (P-value=0.089). In patients affected by ileal or ileocolonic CD, TCRs according to the invention were more abundant as compared to colonic CD (P-value=0.07).

### Clonotypes Comprising the Motif According to the Invention are Present in Both Blood and Intestinal Tissue of CD Patients

To verify the presence and abundance of TCRs according to the invention in CD, an independent sample collection including matched blood and intestinal tissue of 37 individuals undergoing bowel resection surgery (11 CD, 13 UC and 13 colon cancer (CRC) patients as disease controls was analysed; see Example 2). As in the first collection (Example 1), TCRs according to the invention were confirmed to be increased in number and more abundant in CD patients' blood (P-value=0.002). Clonotypes comprising the motif according to the invention were observed in 9/11 (82%) CD, 4/13 (31%) UC and 3/13 (23%) CRC patients' blood. In 8/11 (73%) CD patients, the abundance of TCRs according to the invention was higher than in any other analysed blood sample of the surgery collection. The same trend was observed in intestinal tissue. Clonotypes comprising the motif according to the invention were observed in higher numbers in the blood as compared to the gut. For a subset of patients (4 CD and 5 UC), intestinal mesenteric lymph nodes resected together with the inflamed intestinal tissue were also analysed. While most TCRs according to the invention were observed in the blood, few clonotypes comprising the motif according to the invention were also observed to be present in multiple analysed tissues of the same individual at the same time.

To answer the question whether T-cells comprising the motif according to the invention occur within a particular T cell compartment, blood and intestinal T cells were sorted in CD4+ naïve, CD4+ conventional memory, CD4+ regulatory and CD8+ T cells for 7 CD, 5 UC and 9 CRC additional patients. TCRs according to the invention were found in all analysed fractions, but at different frequencies. Cells according to the invention were most abundant in the CD8+ fraction, particularly in CD patients, with up to 22 TCRs according to the invention detected per individual and higher cumulative abundance as compared to the other cell compartments.

For the three CD patients (Example 7) with a high proportion of cells according to the invention, the CD4+ and CD8+ memory fractions were FACS-purified and single-cell RNA gene expression and TCR sequencing using 10×Genomics' Chromium technology was made. The same analysis was performed for three sex- and age-matched healthy controls.

Cells according to the invention were identified at high abundance (22 to 238 cells per patient) in the three CD patients through their TCRs, while they were rare in healthy controls (0 to 2 CAIT cells per healthy individual). Analysis of TCR alpha/beta pairs showed diverse TCR beta chains associated with the semi-invariant alpha sequences according to the invention. Analysis of the TCR comprising the motif according to the invention alpha/beta chain pairing in clonotypes comprising the motif according to the invention, showed a preferential pairing (38-55% of clonotypes) with beta chains carrying the TRBV7-9 gene in all 3 patients. None of the identified paired TCR beta chains were found in any other individual of the previously analysed bulk TCR datasets (244 whole blood and 58 surgery samples). Thus, these TCR beta sequences seem to be private to single individuals or very rare in the examined population.

### Immunophenotyping of CAIT Cells in CD Patients and Controls

To confirm frequency and phenotype of cells according to the invention identified by TCR and RNA sequencing, we set up an antibody panel for flow cytometry including the antibodies for the TCR V segments TRAV1-2 and TRAV12-1, defining MAIT and CAIT cells respectively, and known unconventional T cell markers such as CD161 (KLRB1) and IL18R. By analysing peripheral blood samples of 20 randomly recruited CD patients and 16 healthy controls, we confirmed that decreased in the blood of CD patients. Cells according to the invention (CD3+TRAV12-1+CD161+IL18R+), were enriched

The frequency difference between patients and controls was observed to be more pronounced in the CD8+ compartment. The same trend for cells according to the invention was observed in the CD4−CD8− and in the CD4+CD8+ fractions.

Using a single-cell transcriptomics approach (see manuscript), it could be showed that the identified T cells seem to have an unconventional innate-like phenotype.

Unconventional T cells are cells not binding the classic MHC alleles but rather MHC-like molecules as MR1 and CD1d. These molecules are less variable among people as compared to classic MHC alleles. Therefore, T cells reactive to MHC-like molecules are also more common among individuals. Therefore, the analysis of these cells and possible drug-discovery research also has higher applicability at population level as compared to a conventional T cell which may be rather private of single individuals.

We also performed a flow cytometry analysis of the T cells. A flow cytometry analysis looks at the protein markers expressed on the surface of the cells and helps to identify the cell phenotype and function. It was found that the cell population including the clonotypes is increased in both CD and UC patients as compared to healthy controls. In the analysis, the cell population was defined to be CD161+TRAV12-1+, where TRAV12-1 is the part of the TCR protein encoded by the TRAV12-1 variable gene.

Because of its shared presence among individuals, these latest results indicate that this population including but not limited to the T cell clonotypes we identified and described in the invention, plays a role in other diseases rather than CD, including UC but potentially also other chronic inflammatory conditions.

1. A protein comprising an amino acid sequence according to SEQ ID NO: 1, wherein the amino acid in position 4 of SEQ ID NO: 1 is selected from the group consisting of N, S, R, T and I, and wherein the amino acid in position 5 of SEQ ID NO: 1 is selected from the group consisting of R, V, L, F, M, I, H, S, T, A, P and G, with the proviso that the amino acid sequence is not SEQ ID NO: 24.

2. The protein of claim 1, wherein the amino acid in position 7 of SEQ ID NO: 1 is A or S.

3. The protein of claim 1, wherein the amino acid sequence according to SEQ ID NO: 1 is selected from the amino acid sequences of any one of SEQ ID NO: 2 to 23 and SEQ ID NO: 25 to 295.

4. The protein of claim 1, wherein the protein is a T-cell receptor (TCR).

5. The protein of claim 4, wherein the amino acid sequence according to SEQ ID NO: 1 is comprised in the alpha chain of the TCR.

6. The protein of claim 5, wherein the amino acid sequence according to SEQ ID NO: 1 is comprised in CDR3 region.

7. A T-cell, comprising the protein of claim 1.

8. The T-cell of claim 7, wherein the T-cell is a human T-cell.

9. The T-cell of claim 7, wherein the T-cell is one or more selected from a CD4+ cell, a CD8+ cell, and and/or a CD161+ cell.

10. A nucleotide sequence encoding an amino acid sequence according to SEQ ID NO: 1, with the proviso that the amino acid sequence is not SEQ ID NO: 24.

11. (canceled)

12. (canceled)

13. A method of obtaining an antibody, comprising raising an antibody against the protein according to claim 1, wherein the antibody is directed against an epitope comprising at least a part of the amino acid sequence according to SEQ ID NO: 1 wherein the amino acid in position 4 of SEQ ID NO: 1 is selected from the group consisting of N, S, R, T and I, and wherein the amino acid in position 5 of SEQ ID NO: 1 is selected from the group consisting of R, V, L, F, M, I, H, S, T, A, P and G.

14. An antibody directed against an epitope comprising at least a part of the amino acid sequence according to SEQ ID NO: 1 wherein the amino acid in position 4 of SEQ ID NO: 1 is selected from the group consisting of N, S, R, T and I, and wherein the amino acid in position 5 of SEQ ID NO: 1 is selected from the group consisting of R, V, L, F, M, I, H, S, T, A, P and G.

15. A method of discriminating blood samples of IBD patients from blood samples of individuals without IBD, comprising quantitatively assessing abundance in the samples of proteins according to claim 1.

16. The method of claim 15, wherein the method discriminates blood samples of Crohn's disease patients from blood samples of individuals without IBD and individuals with IBD other than Crohn's disease.

17. A method of obtaining an antibody, comprising raising an antibody against the T-cell according to claim 7, wherein the antibody is directed against an epitope comprising at least a part of the amino acid sequence according to SEQ ID NO: 1 wherein the amino acid in position 4 of SEQ ID NO: 1 is selected from the group consisting of N, S, R, T and I, and wherein the amino acid in position 5 of SEQ ID NO: 1 is selected from the group consisting of R, V, L, F, M, I, H, S, T, A, P and G.

18. A method of obtaining an antibody, comprising raising an antibody against a protein expressed by a nucleotide sequence according to claim 10, wherein the nucleotide sequence is an mRNA sequence, wherein the antibody is directed against an epitope comprising at least a part of the amino acid sequence according to SEQ ID NO: 1 wherein the amino acid in position 4 of SEQ ID NO: 1 is selected from the group consisting of N, S, R, T and I, and wherein the amino acid in position 5 of SEQ ID NO: 1 is selected from the group consisting of R, V, L, F, M, I, H, S, T, A, P and G.

19. The nucleotide sequence of claim 10, wherein the nucleotide sequence is selected from the DNA sequences of any one of SEQ ID NO: 296 to 1205, except SEQ ID NO: 364, 568, 580, 637, 638, 647, 655, 662, 721, 1020, 1098, 1130, 1137, 1147 and 1150.

20. The nucleotide sequence of claim 10, wherein the nucleotide sequence is an mRNA sequence.


SEQUENCE LISTING (1205 sequences)

SEQ ID NO: 1 - protein, 15 aa, Homo sapiens
  VARIANT 4..5: note = misc_feature - Xaa can be any naturally occurring amino acid
  VARIANT 7: note = misc_feature - Xaa can be any naturally occurring amino acid
CVVXXAXGGSYIPTF

SEQ ID NO: 2 - protein, 15 aa, Homo sapiens
CVVNLASGGSYIPTF

SEQ ID NO: 3 - protein, 15 aa, Homo sapiens
CVVNGASGGSYIPTF

SEQ ID NO: 4 - protein, 15 aa, Homo sapiens
CVVNSASGGSYIPTF

SEQ ID NO: 5 - protein, 15 aa, Homo sapiens
CVVNIASGGSYIPTF

SEQ ID NO: 6 - protein, 15 aa, Homo sapiens
CVVNPASGGSYIPTF

SEQ ID NO: 7 - protein, 15 aa, Homo sapiens
CVVNRASGGSYIPTF

SEQ ID NO: 8 - protein, 15 aa, Homo sapiens
CVVSLASGGSYIPTF

SEQ ID NO: 9 - protein, 15 aa, Homo sapiens
CVVNMASGGSYIPTF

SEQ ID NO: 10 - protein, 15 aa, Homo sapiens
CVVTLASGGSYIPTF

SEQ ID NO: 11 - protein, 15 aa, Homo sapiens
CVVNTASGGSYIPTF

SEQ ID NO: 12 - protein, 15 aa, Homo sapiens
CVVRLASGGSYIPTF

SEQ ID NO: 13 - protein, 15 aa, Homo sapiens
CVVRHASGGSYIPTF

SEQ ID NO: 14 - protein, 15 aa, Homo sapiens
CVVNVASGGSYIPTF

SEQ ID NO: 15 - protein, 15 aa, Homo sapiens
CVVSHASGGSYIPTF

SEQ ID NO: 16 - protein, 15 aa, Homo sapiens
CVVRVASGGSYIPTF

SEQ ID NO: 17 - protein, 15 aa, Homo sapiens
CVVRSASGGSYIPTF

SEQ ID NO: 18 - protein, 15 aa, Homo sapiens
CVVIPASGGSYIPTF

SEQ ID NO: 19 - protein, 15 aa, Homo sapiens
CVVNKASGGSYIPTF

SEQ ID NO: 20 - protein, 15 aa, Homo sapiens
CVVRGASGGSYIPTF

SEQ ID NO: 21 - protein, 15 aa, Homo sapiens
CVVTPASGGSYIPTF

SEQ ID NO: 22 - protein, 15 aa, Homo sapiens
CVVNAASGGSYIPTF

SEQ ID NO: 23 - protein, 15 aa, Homo sapiens
CVVNFASGGSYIPTF

SEQ ID NO: 24 - protein, 15 aa, Homo sapiens
CVVSSASGGSYIPTF

SEQ ID NO: 25 - protein, 15 aa, Homo sapiens
CVVNQASGGSYIPTF

SEQ ID NO: 26 - protein, 15 aa, Homo sapiens
CVVRTASGGSYIPTF

SEQ ID NO: 27 - protein, 15 aa, Homo sapiens
CVVIHASGGSYIPTF

SEQ ID NO: 28 - protein, 15 aa, Homo sapiens
CVVNEASGGSYIPTF

SEQ ID NO: 29 - protein, 15 aa, Homo sapiens
CVVNHASGGSYIPTF

SEQ ID NO: 30 - protein, 15 aa, Homo sapiens
CVVRRASGGSYIPTF

SEQ ID NO: 31 - protein, 15 aa, Homo sapiens
CVVILASGGSYIPTF

SEQ ID NO: 32 - protein, 15 aa, Homo sapiens
CVVRIASGGSYIPTF

SEQ ID NO: 33 - protein, 15 aa, Homo sapiens
CVVRAASGGSYIPTF

SEQ ID NO: 34 - protein, 15 aa, Homo sapiens
CVVSPASGGSYIPTF

SEQ ID NO: 35 - protein, 15 aa, Homo sapiens
CVVSTASGGSYIPTF

SEQ ID NO: 36 - protein, 15 aa, Homo sapiens
CVVTHASGGSYIPTF

SEQ ID NO: 37 - protein, 15 aa, Homo sapiens
CVVTSASGGSYIPTF

SEQ ID NO: 38 - protein, 15 aa, Homo sapiens
CVVTVASGGSYIPTF

SEQ ID NO: 39 - protein, 15 aa, Homo sapiens
CVVNMAAGGSYIPTF

SEQ ID NO: 40 - protein, 15 aa, Homo sapiens
CVVSVASGGSYIPTF

SEQ ID NO: 41 - protein, 15 aa, Homo sapiens
CVVTAASGGSYIPTF

SEQ ID NO: 42 - protein, 15 aa, Homo sapiens
CVVKGASGGSYIPTF

SEQ ID NO: 43 - protein, 15 aa, Homo sapiens
CVVNGAPGGSYIPTF

SEQ ID NO: 44 - protein, 15 aa, Homo sapiens
CVVNIAAGGSYIPTF

SEQ ID NO: 45 - protein, 15 aa, Homo sapiens
CVVNMATGGSYIPTF

SEQ ID NO: 46 - protein, 15 aa, Homo sapiens
CVVPHASGGSYIPTF

SEQ ID NO: 47 - protein, 15 aa, Homo sapiens
CVVSAASGGSYIPTF

SEQ ID NO: 48 - protein, 15 aa, Homo sapiens
CVVTFASGGSYIPTF

SEQ ID NO: 49 - protein, 15 aa, Homo sapiens
CVVREASGGSYIPTF

SEQ ID NO: 50 - protein, 15 aa, Homo sapiens
CVVSFASGGSYIPTF

SEQ ID NO: 51 - protein, 15 aa, Homo sapiens
CVVSGASGGSYIPTF

SEQ ID NO: 52 - protein, 15 aa, Homo sapiens
CVVSIASGGSYIPTF

SEQ ID NO: 53 - protein, 15 aa, Homo sapiens
CVVIAASGGSYIPTF

SEQ ID NO: 54 - protein, 15 aa, Homo sapiens
CVVISASGGSYIPTF

SEQ ID NO: 55 - protein, 15 aa, Homo sapiens
CVVNMAGGGSYIPTF

SEQ ID NO: 56 - protein, 15 aa, Homo sapiens
CVVFHASGGSYIPTF

SEQ ID NO: 57 - protein, 15 aa, Homo sapiens
CVVRQASGGSYIPTF

SEQ ID NO: 58 - protein, 15 aa, Homo sapiens
CVVSRASGGSYIPTF

SEQ ID NO: 59 - protein, 15 aa, Homo sapiens
CVVIGASGGSYIPTF

SEQ ID NO: 60 - protein, 15 aa, Homo sapiens
CVVLHASGGSYIPTF

SEQ ID NO: 61 - protein, 15 aa, Homo sapiens
CVVNIATGGSYIPTF

SEQ ID NO: 62 - protein, 15 aa, Homo sapiens
CVVNYASGGSYIPTF

SEQ ID NO: 63 - protein, 15 aa, Homo sapiens
CVVRFASGGSYIPTF

SEQ ID NO: 64 - protein, 15 aa, Homo sapiens
CVVTLAGGGSYIPTF

SEQ ID NO: 65 - protein, 15 aa, Homo sapiens
CVVAHASGGSYIPTF

SEQ ID NO: 66 - protein, 15 aa, Homo sapiens
CVVIRASGGSYIPTF

SEQ ID NO: 67 - protein, 15 aa, Homo sapiens
CVVNGAAGGSYIPTF

SEQ ID NO: 68 - protein, 15 aa, Homo sapiens
CVVNIAGGGSYIPTF

SEQ ID NO: 69 - protein, 15 aa, Homo sapiens
CVVRMASGGSYIPTF

SEQ ID NO: 70 - protein, 15 aa, Homo sapiens
CVVTIASGGSYIPTF

SEQ ID NO: 71 - protein, 15 aa, Homo sapiens
CVVKHASGGSYIPTF

SEQ ID NO: 72 - protein, 15 aa, Homo sapiens
CVVKSASGGSYIPTF

SEQ ID NO: 73 - protein, 15 aa, Homo sapiens
CVVMGASGGSYIPTF

SEQ ID NO: 74 - protein, 15 aa, Homo sapiens
CVVNDASGGSYIPTF

SEQ ID NO: 75 - protein, 15 aa, Homo sapiens
CVVNRAPGGSYIPTF

SEQ ID NO: 76 - protein, 15 aa, Homo sapiens
CVVTGASGGSYIPTF

SEQ ID NO: 77 - protein, 15 aa, Homo sapiens
CVVALASGGSYIPTF

SEQ ID NO: 78 - protein, 15 aa, Homo sapiens
CVVDHASGGSYIPTF

SEQ ID NO: 79 - protein, 15 aa, Homo sapiens
CVVIFASGGSYIPTF

SEQ ID NO: 80 - protein, 15 aa, Homo sapiens
CVVIIASGGSYIPTF

SEQ ID NO: 81 - protein, 15 aa, Homo sapiens
CVVIVASGGSYIPTF

SEQ ID NO: 82 - protein, 15 aa, Homo sapiens
CVVKPASGGSYIPTF

SEQ ID NO: 83 - protein, 15 aa, Homo sapiens
CVVKRASGGSYIPTF

SEQ ID NO: 84 - protein, 15 aa, Homo sapiens
CVVKTASGGSYIPTF

SEQ ID NO: 85 - protein, 15 aa, Homo sapiens
CVVNLAAGGSYIPTF

SEQ ID NO: 86 - protein, 15 aa, Homo sapiens
CVVNSAAGGSYIPTF

SEQ ID NO: 87 - protein, 15 aa, Homo sapiens
CVVNSAGGGSYIPTF

SEQ ID NO: 88 - protein, 15 aa, Homo sapiens
CVVRKASGGSYIPTF

SEQ ID NO: 89 - protein, 15 aa, Homo sapiens
CVVSQASGGSYIPTF

SEQ ID NO: 90 - protein, 15 aa, Homo sapiens
CVVFLASGGSYIPTF

SEQ ID NO: 91 - protein, 15 aa, Homo sapiens
CVVFSASGGSYIPTF

SEQ ID NO: 92 - protein, 15 aa, Homo sapiens
CVVKAASGGSYIPTF

SEQ ID NO: 93 - protein, 15 aa, Homo sapiens
CVVKLASGGSYIPTF

SEQ ID NO: 94 - protein, 15 aa, Homo sapiens
CVVMHASGGSYIPTF

SEQ ID NO: 95 - protein, 15 aa, Homo sapiens
CVVNLAGGGSYIPTF

SEQ ID NO: 96 - protein, 15 aa, Homo sapiens
CVVNMAPGGSYIPTF

SEQ ID NO: 97 - protein, 15 aa, Homo sapiens
CVVNRAAGGSYIPTF

SEQ ID NO: 98 - protein, 15 aa, Homo sapiens
CVVNRATGGSYIPTF

SEQ ID NO: 99 - protein, 15 aa, Homo sapiens
CVVNVAAGGSYIPTF

SEQ ID NO: 100 - protein, 15 aa, Homo sapiens
CVVNVATGGSYIPTF

SEQ ID NO: 101 - protein, 15 aa, Homo sapiens
CVVNWASGGSYIPTF

SEQ ID NO: 102 - protein, 15 aa, Homo sapiens
CVVSKASGGSYIPTF

SEQ ID NO: 103 - protein, 15 aa, Homo sapiens
CVVSQAAGGSYIPTF

SEQ ID NO: 104 - protein, 15 aa, Homo sapiens
CVVTDASGGSYIPTF

SEQ ID NO: 105 - protein, 15 aa, Homo sapiens
CVVTYASGGSYIPTF

SEQ ID NO: 106 - protein, 15 aa, Homo sapiens
CVVGLASGGSYIPTF

SEQ ID NO: 107 - protein, 15 aa, Homo sapiens
CVVGTASGGSYIPTF

SEQ ID NO: 108 - protein, 15 aa, Homo sapiens
CVVIDASGGSYIPTF

SEQ ID NO: 109 - protein, 15 aa, Homo sapiens
CVVITASGGSYIPTF

SEQ ID NO: 110 - protein, 15 aa, Homo sapiens
CVVKVASGGSYIPTF

SEQ ID NO: 111 - protein, 15 aa, Homo sapiens
CVVLLASGGSYIPTF

SEQ ID NO: 112 - protein, 15 aa, Homo sapiens
CVVNGATGGSYIPTF

SEQ ID NO: 113 - protein, 15 aa, Homo sapiens
CVVNIAPGGSYIPTF

SEQ ID NO: 114 - protein, 15 aa, Homo sapiens
CVVNKAAGGSYIPTF

SEQ ID NO: 115 - protein, 15 aa, Homo sapiens
CVVNLARGGSYIPTF

SEQ ID NO: 116 - protein, 15 aa, Homo sapiens
CVVNSAVGGSYIPTF

SEQ ID NO: 117 - protein, 15 aa, Homo sapiens
CVVNTAGGGSYIPTF

SEQ ID NO: 118 - protein, 15 aa, Homo sapiens
CVVNVAGGGSYIPTF

SEQ ID NO: 119 - protein, 15 aa, Homo sapiens
CVVTQASGGSYIPTF

SEQ ID NO: 120 - protein, 15 aa, Homo sapiens
CVVTTASGGSYIPTF

SEQ ID NO: 121 - protein, 15 aa, Homo sapiens
CVVYHASGGSYIPTF

SEQ ID NO: 122 - protein, 15 aa, Homo sapiens
CVVYLASGGSYIPTF

SEQ ID NO: 123 - protein, 15 aa, Homo sapiens
CVVADASGGSYIPTF

SEQ ID NO: 124 - protein, 15 aa, Homo sapiens
CVVAQASGGSYIPTF

SEQ ID NO: 125 - protein, 15 aa, Homo sapiens
CVVASASGGSYIPTF

SEQ ID NO: 126 - protein, 15 aa, Homo sapiens
CVVATASGGSYIPTF

SEQ ID NO: 127 - protein, 15 aa, Homo sapiens
CVVFGASGGSYIPTF

SEQ ID NO: 128 - protein, 15 aa, Homo sapiens
CVVHHASGGSYIPTF

SEQ ID NO: 129 - protein, 15 aa, Homo sapiens
CVVHVASGGSYIPTF

SEQ ID NO: 130 - protein, 15 aa, Homo sapiens
CVVINASGGSYIPTF

SEQ ID NO: 131 - protein, 15 aa, Homo sapiens
CVVIQASGGSYIPTF

SEQ ID NO: 132 - protein, 15 aa, Homo sapiens
CVVKEASGGSYIPTF

SEQ ID NO: 133 - protein, 15 aa, Homo sapiens
CVVKIASGGSYIPTF

SEQ ID NO: 134 - protein, 15 aa, Homo sapiens
CVVKKASGGSYIPTF

SEQ ID NO: 135 - protein, 15 aa, Homo sapiens
CVVKMASGGSYIPTF

SEQ ID NO: 136 - protein, 15 aa, Homo sapiens
CVVKRAGGGSYIPTF

SEQ ID NO: 137 - protein, 15 aa, Homo sapiens
CVVLGASGGSYIPTF

SEQ ID NO: 138 - protein, 15 aa, Homo sapiens
CVVLPASGGSYIPTF

SEQ ID NO: 139 - protein, 15 aa, Homo sapiens
CVVLSASGGSYIPTF

SEQ ID NO: 140 - protein, 15 aa, Homo sapiens
CVVLTASGGSYIPTF

SEQ ID NO: 141 - protein, 15 aa, Homo sapiens
CVVMPASGGSYIPTF

SEQ ID NO: 142 - protein, 15 aa, Homo sapiens
CVVNEAAGGSYIPTF

SEQ ID NO: 143 - protein, 15 aa, Homo sapiens
CVVNIALGGSYIPTF

SEQ ID NO: 144 - protein, 15 aa, Homo sapiens
CVVNIARGGSYIPTF

SEQ ID NO: 145 - protein, 15 aa, Homo sapiens
CVVNMAEGGSYIPTF

SEQ ID NO: 146 - protein, 15 aa, Homo sapiens
CVVNPATGGSYIPTF

SEQ ID NO: 147 - protein, 15 aa, Homo sapiens
CVVNSATGGSYIPTF

SEQ ID NO: 148 - protein, 15 aa, Homo sapiens
CVVPFASGGSYIPTF

SEQ ID NO: 149 - protein, 15 aa, Homo sapiens
CVVPPASGGSYIPTF

SEQ ID NO: 150 - protein, 15 aa, Homo sapiens
CVVPRASGGSYIPTF

SEQ ID NO: 151 - protein, 15 aa, Homo sapiens
CVVPSASGGSYIPTF

SEQ ID NO: 152 - protein, 15 aa, Homo sapiens
CVVPTASGGSYIPTF

SEQ ID NO: 153 - protein, 15 aa, Homo sapiens
CVVRLAGGGSYIPTF

SEQ ID NO: 154 - protein, 15 aa, Homo sapiens
CVVRPASGGSYIPTF

SEQ ID NO: 155 - protein, 15 aa, Homo sapiens
CVVRTARGGSYIPTF

SEQ ID NO: 156 - protein, 15 aa, Homo sapiens
CVVRVAAGGSYIPTF

SEQ ID NO: 157 - protein, 15 aa, Homo sapiens
CVVRYASGGSYIPTF

SEQ ID NO: 158 - protein, 15 aa, Homo sapiens
CVVSDASGGSYIPTF

SEQ ID NO: 159 - protein, 15 aa, Homo sapiens
CVVSGAVGGSYIPTF

SEQ ID NO: 160 - protein, 15 aa, Homo sapiens
CVVSLAGGGSYIPTF

SEQ ID NO: 161 - protein, 15 aa, Homo sapiens
CVVVHASGGSYIPTF

SEQ ID NO: 162 - protein, 15 aa, Homo sapiens
CVVVLASGGSYIPTF

SEQ ID NO: 163 - protein, 15 aa, Homo sapiens
CVVAFASGGSYIPTF

SEQ ID NO: 164 - protein, 15 aa, Homo sapiens
CVVAGASGGSYIPTF

SEQ ID NO: 165 - protein, 15 aa, Homo sapiens
CVVAPASGGSYIPTF

SEQ ID NO: 166 - protein, 15 aa, Homo sapiens
CVVAVASGGSYIPTF

SEQ ID NO: 167 - protein, 15 aa, Homo sapiens
CVVAWASGGSYIPTF

SEQ ID NO: 168 - protein, 15 aa, Homo sapiens
CVVDDASGGSYIPTF

SEQ ID NO: 169 - protein, 15 aa, Homo sapiens
CVVDQASGGSYIPTF

SEQ ID NO: 170 - protein, 15 aa, Homo sapiens
CVVEQASGGSYIPTF

SEQ ID NO: 171 - protein, 15 aa, Homo sapiens
CVVFAASGGSYIPTF

SEQ ID NO: 172 - protein, 15 aa, Homo sapiens
CVVFLAGGGSYIPTF

SEQ ID NO: 173 - protein, 15 aa, Homo sapiens
CVVFQARGGSYIPTF

SEQ ID NO: 174 - protein, 15 aa, Homo sapiens
CVVFQASGGSYIPTF

SEQ ID NO: 175 - protein, 15 aa, Homo sapiens
CVVFRASGGSYIPTF

SEQ ID NO: 176 - protein, 15 aa, Homo sapiens
CVVFTASGGSYIPTF

SEQ ID NO: 177 - protein, 15 aa, Homo sapiens
CVVFVASGGSYIPTF

SEQ ID NO: 178 - protein, 15 aa, Homo sapiens
CVVGDASGGSYIPTF

SEQ ID NO: 179 - protein, 15 aa, Homo sapiens
CVVGIASGGSYIPTF

SEQ ID NO: 180 - protein, 15 aa, Homo sapiens
CVVGRASGGSYIPTF

SEQ ID NO: 181 - protein, 15 aa, Homo sapiens
CVVGVAAGGSYIPTF

SEQ ID NO: 182 - protein, 15 aa, Homo sapiens
CVVGVASGGSYIPTF

SEQ ID NO: 183 - protein, 15 aa, Homo sapiens
CVVHDASGGSYIPTF

SEQ ID NO: 184 - protein, 15 aa, Homo sapiens
CVVHEASGGSYIPTF

SEQ ID NO: 185 - protein, 15 aa, Homo sapiens
CVVHLASGGSYIPTF

SEQ ID NO: 186 - protein, 15 aa, Homo sapiens
CVVHQASGGSYIPTF

SEQ ID NO: 187 - protein, 15 aa, Homo sapiens
CVVHYASGGSYIPTF

SEQ ID NO: 188 - protein, 15 aa, Homo sapiens
CVVIPATGGSYIPTF

SEQ ID NO: 189 - protein, 15 aa, Homo sapiens
CVVISAPGGSYIPTF

SEQ ID NO: 190 - protein, 15 aa, Homo sapiens
CVVIYASGGSYIPTF

SEQ ID NO: 191 - protein, 15 aa, Homo sapiens
CVVKAAAGGSYIPTF

SEQ ID NO: 192 - protein, 15 aa, Homo sapiens
CVVKDASGGSYIPTF

SEQ ID NO: 193 - protein, 15 aa, Homo sapiens
CVVKFASGGSYIPTF

SEQ ID NO: 194 - protein, 15 aa, Homo sapiens
CVVKGAGGGSYIPTF

SEQ ID NO: 195 - protein, 15 aa, Homo sapiens
CVVKGAPGGSYIPTF

SEQ ID NO: 196 - protein, 15 aa, Homo sapiens
CVVKLAVGGSYIPTF

SEQ ID NO: 197 - protein, 15 aa, Homo sapiens
CVVKNASGGSYIPTF

SEQ ID NO: 198 - protein, 15 aa, Homo sapiens
CVVKSAGGGSYIPTF

SEQ ID NO: 199 - protein, 15 aa, Homo sapiens
CVVKTAAGGSYIPTF

SEQ ID NO: 200 - protein, 15 aa, Homo sapiens
CVVKTAGGGSYIPTF

SEQ ID NO: 201 - protein, 15 aa, Homo sapiens
CVVKVAGGGSYIPTF

SEQ ID NO: 202 - protein, 15 aa, Homo sapiens
CVVKYASGGSYIPTF

SEQ ID NO: 203 - protein, 15 aa, Homo sapiens
CVVLGAAGGSYIPTF

SEQ ID NO: 204 - protein, 15 aa, Homo sapiens
CVVLVASGGSYIPTF

SEQ ID NO: 205 - protein, 15 aa, Homo sapiens
CVVMAASGGSYIPTF

SEQ ID NO: 206 - protein, 15 aa, Homo sapiens
CVVMKAEGGSYIPTF

SEQ ID NO: 207 - protein, 15 aa, Homo sapiens
CVVMLASGGSYIPTF

SEQ ID NO: 208 - protein, 15 aa, Homo sapiens
CVVMRASGGSYIPTF

SEQ ID NO: 209 - protein, 15 aa, Homo sapiens
CVVMTASGGSYIPTF

SEQ ID NO: 210 - protein, 15 aa, Homo sapiens
CVVNAAAGGSYIPTF

SEQ ID NO: 211 - protein, 15 aa, Homo sapiens
CVVNAAPGGSYIPTF

SEQ ID NO: 212 - protein, 15 aa, Homo sapiens
CVVNAARGGSYIPTF

SEQ ID NO: 213 - protein, 15 aa, Homo sapiens
CVVNAATGGSYIPTF

SEQ ID NO: 214 - protein, 15 aa, Homo sapiens
CVVNEAGGGSYIPTF

SEQ ID NO: 215 - protein, 15 aa, Homo sapiens
CVVNEATGGSYIPTF

SEQ ID NO: 216 - protein, 15 aa, Homo sapiens
CVVNGAGGGSYIPTF

SEQ ID NO: 217 - protein, 15 aa, Homo sapiens
CVVNGAQGGSYIPTF

SEQ ID NO: 218 - protein, 15 aa, Homo sapiens
CVVNGARGGSYIPTF

SEQ ID NO: 219 - protein, 15 aa, Homo sapiens
CVVNGAVGGSYIPTF

SEQ ID NO: 220 - protein, 15 aa, Homo sapiens
CVVNIAEGGSYIPTF

SEQ ID NO: 221 - protein, 15 aa, Homo sapiens
CVVNIAIGGSYIPTF

SEQ ID NO: 222 - protein, 15 aa, Homo sapiens
CVVNKAKGGSYIPTF

SEQ ID NO: 223 - protein, 15 aa, Homo sapiens
CVVNKAPGGSYIPTF

SEQ ID NO: 224 - protein, 15 aa, Homo sapiens
CVVNKATGGSYIPTF

SEQ ID NO: 225 - protein, 15 aa, Homo sapiens
CVVNLAVGGSYIPTF

SEQ ID NO: 226 - protein, 15 aa, Homo sapiens
CVVNMALGGSYIPTF

SEQ ID NO: 227 - protein, 15 aa, Homo sapiens
CVVNMAQGGSYIPTF

SEQ ID NO: 228 - protein, 15 aa, Homo sapiens
CVVNMARGGSYIPTF

SEQ ID NO: 229 - protein, 15 aa, Homo sapiens
CVVNNAPGGSYIPTF

SEQ ID NO: 230 - protein, 15 aa, Homo sapiens
CVVNNARGGSYIPTF

SEQ ID NO: 231 - protein, 15 aa, Homo sapiens
CVVNNASGGSYIPTF

SEQ ID NO: 232 - protein, 15 aa, Homo sapiens
CVVNPALGGSYIPTF

SEQ ID NO: 233 - protein, 15 aa, Homo sapiens
CVVNPAPGGSYIPTF

SEQ ID NO: 234 - protein, 15 aa, Homo sapiens
CVVNQAAGGSYIPTF

SEQ ID NO: 235 - protein, 15 aa, Homo sapiens
CVVNQATGGSYIPTF

SEQ ID NO: 236 - protein, 15 aa, Homo sapiens
CVVNRAGGGSYIPTF

SEQ ID NO: 237 - protein, 15 aa, Homo sapiens
CVVNSAEGGSYIPTF

SEQ ID NO: 238 - protein, 15 aa, Homo sapiens
CVVNSAKGGSYIPTF

SEQ ID NO: 239 - protein, 15 aa, Homo sapiens
CVVNTAAGGSYIPTF

SEQ ID NO: 240 - protein, 15 aa, Homo sapiens
CVVNTAKGGSYIPTF

SEQ ID NO: 241 - protein, 15 aa, Homo sapiens
CVVNTAPGGSYIPTF

SEQ ID NO: 242 - protein, 15 aa, Homo sapiens
CVVNTARGGSYIPTF

SEQ ID NO: 243 - protein, 15 aa, Homo sapiens
CVVNVAPGGSYIPTF

SEQ ID NO: 244 - protein, 15 aa, Homo sapiens
CVVNWAAGGSYIPTF

SEQ ID NO: 245 - protein, 15 aa, Homo sapiens
CVVNWAGGGSYIPTF

SEQ ID NO: 246 - protein, 15 aa, Homo sapiens
CVVPEASGGSYIPTF

SEQ ID NO: 247 - protein, 15 aa, Homo sapiens
CVVPGASGGSYIPTF

SEQ ID NO: 248 - protein, 15 aa, Homo sapiens
CVVPLASGGSYIPTF

SEQ ID NO: 249 - protein, 15 aa, Homo sapiens
CVVPLATGGSYIPTF

SEQ ID NO: 250 - protein, 15 aa, Homo sapiens
CVVPPAPGGSYIPTF

SEQ ID NO: 251 - protein, 15 aa, Homo sapiens
CVVPVASGGSYIPTF

SEQ ID NO: 252 - protein, 15 aa, Homo sapiens
CVVQGASGGSYIPTF

SEQ ID NO: 253 - protein, 15 aa, Homo sapiens
CVVQHASGGSYIPTF

SEQ ID NO: 254 - protein, 15 aa, Homo sapiens
CVVQLASGGSYIPTF

SEQ ID NO: 255 - protein, 15 aa, Homo sapiens
CVVQSASGGSYIPTF

SEQ ID NO: 256 - protein, 15 aa, Homo sapiens
CVVRDASGGSYIPTF

SEQ ID NO: 257 - protein, 15 aa, Homo sapiens
CVVREAWGGSYIPTF

SEQ ID NO: 258 - protein, 15 aa, Homo sapiens
CVVRFARGGSYIPTF

SEQ ID NO: 259 - protein, 15 aa, Homo sapiens
CVVRGAEGGSYIPTF

SEQ ID NO: 260 - protein, 15 aa, Homo sapiens
CVVRHATGGSYIPTF

SEQ ID NO: 261 - protein, 15 aa, Homo sapiens
CVVRQAGGGSYIPTF

SEQ ID NO: 262 - protein, 15 aa, Homo sapiens
CVVRRAAGGSYIPTF

SEQ ID NO: 263 - protein, 15 aa, Homo sapiens
CVVRSATGGSYIPTF

SEQ ID NO: 264 - protein, 15 aa, Homo sapiens
CVVRTAQGGSYIPTF

SEQ ID NO: 265 - protein, 15 aa, Homo sapiens
CVVRVAGGGSYIPTF

SEQ ID NO: 266 - protein, 15 aa, Homo sapiens
CVVSEASGGSYIPTF

SEQ ID NO: 267 - protein, 15 aa, Homo sapiens
CVVSGAQGGSYIPTF

SEQ ID NO: 268 - protein, 15 aa, Homo sapiens
CVVSLAAGGSYIPTF

SEQ ID NO: 269 - protein, 15 aa, Homo sapiens
CVVSLAKGGSYIPTF

SEQ ID NO: 270 - protein, 15 aa, Homo sapiens
CVVSMASGGSYIPTF

SEQ ID NO: 271 - protein, 15 aa, Homo sapiens
CVVSPAPGGSYIPTF

SEQ ID NO: 272 - protein, 15 aa, Homo sapiens
CVVSQAGGGSYIPTF

SEQ ID NO: 273 - protein, 15 aa, Homo sapiens
CVVSRAMGGSYIPTF

SEQ ID NO: 274 - protein, 15 aa, Homo sapiens
CVVSSAVGGSYIPTF

SEQ ID NO: 275 - protein, 15 aa, Homo sapiens
CVVSTATGGSYIPTF

SEQ ID NO: 276 - protein, 15 aa, Homo sapiens
CVVTEASGGSYIPTF

SEQ ID NO: 277 - protein, 15 aa, Homo sapiens
CVVTGAAGGSYIPTF

SEQ ID NO: 278 - protein, 15 aa, Homo sapiens
CVVTIAEGGSYIPTF

SEQ ID NO: 279 - protein, 15 aa, Homo sapiens
CVVTNAPGGSYIPTF

SEQ ID NO: 280 - protein, 15 aa, Homo sapiens
CVVTPAPGGSYIPTF

SEQ ID NO: 281 - protein, 15 aa, Homo sapiens
CVVTRASGGSYIPTF

SEQ ID NO: 282 - protein, 15 aa, Homo sapiens
CVVTSARGGSYIPTF

SEQ ID NO: 283 - protein, 15 aa, Homo sapiens
CVVTSATGGSYIPTF

SEQ ID NO: 284 - protein, 15 aa, Homo sapiens
CVVTTAGGGSYIPTF

SEQ ID NO: 285 - protein, 15 aa, Homo sapiens
CVVVFASGGSYIPTF

SEQ ID NO: 286 - protein, 15 aa, Homo sapiens
CVVVGASGGSYIPTF

SEQ ID NO: 287 - protein, 15 aa, Homo sapiens
CVVVMASGGSYIPTF

SEQ ID NO: 288 - protein, 15 aa, Homo sapiens
CVVVRASGGSYIPTF

SEQ ID NO: 289 - protein, 15 aa, Homo sapiens
CVVWGASGGSYIPTF

SEQ ID NO: 290 - protein, 15 aa, Homo sapiens
CVVWPASGGSYIPTF

SEQ ID NO: 291 - protein, 15 aa, Homo sapiens
CVVWTASGGSYIPTF

SEQ ID NO: 292 - protein, 15 aa, Homo sapiens
CVVYFASGGSYIPTF

SEQ ID NO: 293 - protein, 15 aa, Homo sapiens
CVVYGASGGSYIPTF

SEQ ID NO: 294 - protein, 15 aa, Homo sapiens
CVVYMAPGGSYIPTF

SEQ ID NO: 295 - protein, 15 aa, Homo sapiens
CVVYPASGGSYIPTF

SEQ ID NO: 296 - DNA, 45 bp, Homo sapiens
  source 1..45: mol_type = genomic DNA
tgtgtggtgaacattgcatcaggaggaagctacatacctacattt

SEQ ID NO: 297 - DNA, 45 bp, Homo sapiens
  source 1..45: mol_type = genomic DNA
tgtgtggtgaacttggcatcaggaggaagctacatacctacattt

SEQ ID NO: 298 - DNA, 45 bp, Homo sapiens
  source 1..45: mol_type = genomic DNA
tgtgtggtgaacaaggcggcaggaggaagctacatacctacattt

SEQ ID NO: 299 - DNA, 45 bp, Homo sapiens
  source 1..45: mol_type = genomic DNA
tgtgtggtccgaattgcatcaggaggaagctacatacctacattt

SEQ ID NO: 300 - DNA, 45 bp, Homo sapiens
  source 1..45: mol_type = genomic DNA
tgtgtggtgattcatgcatcaggaggaagctacatacctacattt

SEQ ID NO: 301 - DNA, 45 bp, Homo sapiens
  source 1..45: mol_type = genomic DNA
tgtgtggtgaggggagcatcaggaggaagctacatacctacattt

SEQ ID NO: 302 - DNA, 45 bp, Homo sapiens
  source 1..45: mol_type = genomic DNA
tgtgtggtgagaaaggcgtcaggaggaagctacatacctacattt

SEQ ID NO: 303 - DNA, 45 bp, Homo sapiens
  source 1..45: mol_type = genomic DNA
tgtgtggtgaacttcgcatcaggaggaagctacatacctacattt

SEQ ID NO: 304 - DNA, 45 bp, Homo sapiens
  source 1..45: mol_type = genomic DNA
tgtgtggtgaaccttgcatcaggaggaagctacatacctacattt

SEQ ID NO: 305 - DNA, 45 bp, Homo sapiens
  source 1..45: mol_type = genomic DNA
tgtgtggtgaacctcgcatcaggaggaagctacatacctacattt

SEQ ID NO: 306 - DNA, 45 bp, Homo sapiens
  source 1..45: mol_type = genomic DNA
tgtgtggtgaacacggcatcaggaggaagctacatacctacattt

SEQ ID NO: 307 - DNA, 45 bp, Homo sapiens
  source 1..45: mol_type = genomic DNA
tgtgtggtgaacagagccccaggaggaagctacatacctacattt

SEQ ID NO: 308 - DNA, 45 bp, Homo sapiens
  source 1..45: mol_type = genomic DNA
tgtgtggtgagggtggcatcaggaggaagctacatacctacattt

SEQ ID NO: 309 - DNA, 45 bp, Homo sapiens
  source 1..45: mol_type = genomic DNA
tgtgtggtccgtttcgcatcaggaggaagctacatacctacattt

SEQ ID NO: 310 - DNA, 45 bp, Homo sapiens
  source 1..45: mol_type = genomic DNA
tgtgtggtgaccgtagcatcaggaggaagctacatacctacattt

SEQ ID NO: 311 - DNA, 45 bp, Homo sapiens
  source 1..45: mol_type = genomic DNA
tgtgtggtgaacgccgcatcaggaggaagctacatacctacattt

SEQ ID NO: 312 - DNA, 45 bp, Homo sapiens
  source 1..45: mol_type = genomic DNA
tgtgtggtgaacatcgcatcaggaggaagctacatacctacattt

SEQ ID NO: 313 - DNA, 45 bp, Homo sapiens
  source 1..45: mol_type = genomic DNA
tgtgtggtgaacggcgcatcaggaggaagctacatacctacattt

SEQ ID NO: 314 - DNA, 45 bp, Homo sapiens
  source 1..45: mol_type = genomic DNA
tgtgtggtgagccttgcatcaggaggaagctacatacctacattt

SEQ ID NO: 315 - DNA, 45 bp, Homo sapiens
  source 1..45: mol_type = genomic DNA
tgtgtggttcgttttgcatcaggaggaagctacatacctacattt

SEQ ID NO: 316 - DNA, 45 bp, Homo sapiens
  source 1..45: mol_type = genomic DNA
tgtgtggtgaggtttgcatcaggaggaagctacatacctacattt

SEQ ID NO: 317 - DNA, 45 bp, Homo sapiens
  source 1..45: mol_type = genomic DNA
tgtgtggtgcgttcggcatcaggaggaagctacatacctacattt

SEQ ID NO: 318 - DNA, 45 bp, Homo sapiens
  source 1..45: mol_type = genomic DNA
tgtgtggtgagcttagcatcaggaggaagctacatacctacattt

SEQ ID NO: 319 - DNA, 45 bp, Homo sapiens
  source 1..45: mol_type = genomic DNA
tgtgtggttaaacttgcatcaggaggaagctacatacctacattt

SEQ ID NO: 320 - DNA, 45 bp, Homo sapiens
  source 1..45: mol_type = genomic DNA
tgtgtggtgaactttgcatcaggaggaagctacatacctacattt

SEQ ID NO: 321 - DNA, 45 bp, Homo sapiens
  source 1..45: mol_type = genomic DNA
tgtgtggtgaggctcgcatcaggaggaagctacatacctacattt

SEQ ID NO: 322 - DNA, 45 bp, Homo sapiens
  source 1..45: mol_type = genomic DNA
tgtgtggtgagggtcgcatcaggaggaagctacatacctacattt

SEQ ID NO: 323 - DNA, 45 bp, Homo sapiens
  source 1..45: mol_type = genomic DNA
tgtgtggtgaggcatgcatcaggaggaagctacatacctacattt

SEQ ID NO: 324 - DNA, 45 bp, Homo sapiens
  source 1..45: mol_type = genomic DNA
tgtgtggtgaggaccgcatcaggaggaagctacatacctacattt

SEQ ID NO: 325 - DNA, 45 bp, Homo sapiens
  source 1..45: mol_type = genomic DNA
tgtgtggtgagagccgcatcaggaggaagctacatacctacattt

SEQ ID NO: 326 - DNA, 45 bp, Homo sapiens
  source 1..45: mol_type = genomic DNA
tgtgtggtgaacaccgcatcaggaggaagctacatacctacattt

SEQ ID NO: 327 - DNA, 45 bp, Homo sapiens
  source 1..45: mol_type = genomic DNA
tgtgtggtccgacgggcatcaggaggaagctacatacctacattt

SEQ ID NO: 328 - DNA, 45 bp, Homo sapiens
  source 1..45: mol_type = genomic DNA
tgtgtggtgactttagcatcaggaggaagctacatacctacattt

SEQ ID NO: 329 - DNA, 45 bp, Homo sapiens
  source 1..45: mol_type = genomic DNA
tgtgtggtgacgttcgcatcaggaggaagctacatacctacattt

SEQ ID NO: 330 - DNA, 45 bp, Homo sapiens
  source 1..45: mol_type = genomic DNA
tgtgtggtgagcgtggcatcaggaggaagctacatacctacattt

SEQ ID NO: 331 - DNA, 45 bp, Homo sapiens
  source 1..45: mol_type = genomic DNA
tgtgtggtgaggggggcatcaggaggaagctacatacctacattt

SEQ ID NO: 332 - DNA, 45 bp, Homo sapiens
  source 1..45: mol_type = genomic DNA
tgtgtggtgaggacggcatcaggaggaagctacatacctacattt

SEQ ID NO: 333 - DNA, 45 bp, Homo sapiens
  source 1..45: mol_type = genomic DNA
tgtgtggtgaactctgcatcaggaggaagctacatacctacattt

SEQ ID NO: 334 - DNA, 45 bp, Homo sapiens
  source 1..45: mol_type = genomic DNA
tgtgtggtgaactcggctggaggaggaagctacatacctacattt

SEQ ID NO: 335 - DNA, 45 bp, Homo sapiens
  source 1..45: mol_type = genomic DNA
tgtgtggtgaaccttgcctcaggaggaagctacatacctacattt

SEQ ID NO: 336 - DNA, 45 bp, Homo sapiens
  source 1..45: mol_type = genomic DNA
tgtgtggtgaacatggcatcaggaggaagctacatacctacattt

SEQ ID NO: 337 - DNA, 45 bp, Homo sapiens
  source 1..45: mol_type = genomic DNA
tgtgtggtgaacagggcatcaggaggaagctacatacctacattt

SEQ ID NO: 338 - DNA, 45 bp, Homo sapiens
  source 1..45: mol_type = genomic DNA
tgtgtggtgaacacagcggcaggaggaagctacatacctacattt

SEQ ID NO: 339 - DNA, 45 bp, Homo sapiens
  source 1..45: mol_type = genomic DNA
tgtgtggtgaatcttgcatcaggaggaagctacatacctacattt

SEQ ID NO: 340 - DNA, 45 bp, Homo sapiens
  source 1..45: mol_type = genomic DNA
tgtgtggtgaccgtcgcatcaggaggaagctacatacctacattt

SEQ ID NO: 341 - DNA, 45 bp, Homo sapiens
  source 1..45: mol_type = genomic DNA
tgtgtggtgaggagcgccacaggaggaagctacatacctacattt

SEQ ID NO: 342 - DNA, 45 bp, Homo sapiens
  source 1..45: mol_type = genomic DNA
tgtgtggtgaacgtagcatcaggaggaagctacatacctacattt

SEQ ID NO: 343 - DNA, 45 bp, Homo sapiens
  source 1..45: mol_type = genomic DNA
tgtgtggtgaacatagcatcaggaggaagctacatacctacattt

SEQ ID NO: 344 - DNA, 45 bp, Homo sapiens
  source 1..45: mol_type = genomic DNA
tgtgtggtccgccaggcatcaggaggaagctacatacctacattt

SEQ ID NO: 345 - DNA, 45 bp, Homo sapiens
  source 1..45: mol_type = genomic DNA
tgtgtggtgacctttgcatcaggaggaagctacatacctacattt

SEQ ID NO: 346 - DNA, 45 bp, Homo sapiens
  source 1..45: mol_type = genomic DNA
tgtgtggtgagcctggcaaagggaggaagctacatacctacattt

SEQ ID NO: 347 - DNA, 45 bp, Homo sapiens
  source 1..45: mol_type = genomic DNA
tgtgtggtgagcaccgcttcaggaggaagctacatacctacattt

SEQ ID NO: 348 - DNA, 45 bp, Homo sapiens
  source 1..45: mol_type = genomic DNA
tgtgtggtgaggcgtgcatcaggaggaagctacatacctacattt

SEQ ID NO: 349 - DNA, 45 bp, Homo sapiens
  source 1..45: mol_type = genomic DNA
tgtgtggtgaggagtgcatcaggaggaagctacatacctacattt

SEQ ID NO: 350 - DNA, 45 bp, Homo sapiens
  source 1..45: mol_type = genomic DNA
tgtgtggtgaacctggcggggggaggaagctacatacctacattt

SEQ ID NO: 351 - DNA, 45 bp, Homo sapiens
  source 1..45: mol_type = genomic DNA
tgtgtggtgaacggcgccgcaggaggaagctacatacctacattt

SEQ ID NO: 352 - DNA, 45 bp, Homo sapiens
  source 1..45: mol_type = genomic DNA
tgtgtggtgaaccgggcatcaggaggaagctacatacctacattt

SEQ ID NO: 353 - DNA, 45 bp, Homo sapiens
  source 1..45: mol_type = genomic DNA
tgtgtggtgaacatggcgggaggaggaagctacatacctacattt

SEQ ID NO: 354 - DNA, 45 bp, Homo sapiens
  source 1..45: mol_type = genomic DNA
tgtgtggtgaccctggcatcaggaggaagctacatacctacattt

SEQ ID NO: 355 - DNA, 45 bp, Homo sapiens
  source 1..45: mol_type = genomic DNA
tgtgtggtccgagttgcatcaggaggaagctacatacctacattt

SEQ ID NO: 356 - DNA, 45 bp, Homo sapiens
  source 1..45: mol_type = genomic DNA
tgtgtggtgaccaccgcatcaggaggaagctacatacctacattt

SEQ ID NO: 357 - DNA, 45 bp, Homo sapiens
  source 1..45: mol_type = genomic DNA
tgtgtggtgagcttcgcatcaggaggaagctacatacctacattt

SEQ ID NO: 358 - DNA, 45 bp, Homo sapiens
  source 1..45: mol_type = genomic DNA
tgtgtggtgggcctagcatcaggaggaagctacatacctacattt

SEQ ID NO: 359 - DNA, 45 bp, Homo sapiens
  source 1..45: mol_type = genomic DNA
tgtgtggtggggatagcatcaggaggaagctacatacctacattt

SEQ ID NO: 360 - DNA, 45 bp, Homo sapiens
  source 1..45: mol_type = genomic DNA
tgtgtggtgaatctcgcatcaggaggaagctacatacctacattt

SEQ ID NO: 361 - DNA, 45 bp, Homo sapiens
  source 1..45: mol_type = genomic DNA
tgtgtggtgaacctggcatcaggaggaagctacatacctacattt

SEQ ID NO: 362 - DNA, 45 bp, Homo sapiens
  source 1..45: mol_type = genomic DNA
tgtgtggtgaacagtgcatcaggaggaagctacatacctacattt

SEQ ID NO: 363 - DNA, 45 bp, Homo sapiens
  source 1..45: mol_type = genomic DNA
tgtgtggtacggtcggcatcaggaggaagctacatacctacattt

SEQ ID NO: 364 - DNA, 45 bp, Homo sapiens
  source 1..45: mol_type = genomic DNA
tgtgtggtctcgtctgcatcaggaggaagctacatacctacattt

SEQ ID NO: 365 - DNA, 45 bp, Homo sapiens
  source 1..45: mol_type = genomic DNA
tgtgtggtgcgtctggcatcaggaggaagctacatacctacattt

SEQ ID NO: 366 - DNA, 45 bp, Homo sapiens
  source 1..45: mol_type = genomic DNA
tgtgtggtgcgtaccgcatcaggaggaagctacatacctacattt

SEQ ID NO: 367 - DNA, 45 bp, Homo sapiens
  source 1..45: mol_type = genomic DNA
tgtgtggtgacggtcgcatcaggaggaagctacatacctacattt

SEQ ID NO: 368 - DNA, 45 bp, Homo sapiens
  source 1..45: mol_type = genomic DNA
tgtgtggtgaggtccgcatcaggaggaagctacatacctacattt

SEQ ID NO: 369 - DNA, 45 bp, Homo sapiens
  source 1..45: mol_type = genomic DNA
tgtgtggtgaggctagcatcaggaggaagctacatacctacattt

SEQ ID NO: 370 - DNA, 45 bp, Homo sapiens
  source 1..45: mol_type = genomic DNA
tgtgtggtgaacatggcgacaggaggaagctacatacctacattt

SEQ ID NO: 371 - DNA, 45 bp, Homo sapiens
  source 1..45: mol_type = genomic DNA
tgtgtggtgtccgaggcctcaggaggaagctacatacctacattt

SEQ ID NO: 372 - DNA, 45 bp, Homo sapiens
  source 1..45: mol_type = genomic DNA
tgtgtggtgtcgatcgcatcaggaggaagctacatacctacattt

SEQ ID NO: 373 - DNA, 45 bp, Homo sapiens
  source 1..45: mol_type = genomic DNA
tgtgtggtgactctcgcatcaggaggaagctacatacctacattt

SEQ ID NO: 374 - DNA, 45 bp, Homo sapiens
  source 1..45: mol_type = genomic DNA
tgtgtggtgacccttgcatcaggaggaagctacatacctacattt

SEQ ID NO: 375 - DNA, 45 bp, Homo sapiens
  source 1..45: mol_type = genomic DNA
tgtgtggtgaccctggcggggggaggaagctacatacctacattt

SEQ ID NO: 376 - DNA, 45 bp, Homo sapiens
  source 1..45: mol_type = genomic DNA
tgtgtggtgacgctagcatcaggaggaagctacatacctacattt

SEQ ID NO: 377 - DNA, 45 bp, Homo sapiens
  source 1..45: mol_type = genomic DNA
tgtgtggtgagcccagcatcaggaggaagctacatacctacattt

SEQ ID NO: 378 - DNA, 45 bp, Homo sapiens
  source 1..45: mol_type = genomic DNA
tgtgtggtgaggttagcatcaggaggaagctacatacctacattt

SEQ ID NO: 379 - DNA, 45 bp, Homo sapiens
  source 1..45: mol_type = genomic DNA
tgtgtggtgaggctggcatcaggaggaagctacatacctacattt

SEQ ID NO: 380 - DNA, 45 bp, Homo sapiens
  source 1..45: mol_type = genomic DNA
tgtgtggtgagggttgcatcaggaggaagctacatacctacattt

SEQ ID NO: 381 - DNA, 45 bp, Homo sapiens
  source 1..45: mol_type = genomic DNA
tgtgtggtgaggatcgcatcaggaggaagctacatacctacattt

SEQ ID NO: 382 - DNA, 45 bp, Homo sapiens
  source 1..45: mol_type = genomic DNA
tgtgtggtgagacatgcatcaggaggaagctacatacctacattt

SEQ ID NO: 383 - DNA, 45 bp, Homo sapiens
  source 1..45: mol_type = genomic DNA
tgtgtggtgaatgttgcatcaggaggaagctacatacctacattt

SEQ ID NO: 384 - DNA, 45 bp, Homo sapiens
  source 1..45: mol_type = genomic DNA
tgtgtggtgaactctgccgcaggaggaagctacatacctacattt

SEQ ID NO: 385 - DNA, 45 bp, Homo sapiens
  source 1..45: mol_type = genomic DNA
tgtgtggtgaacgttgcatcaggaggaagctacatacctacattt

SEQ ID NO: 386 - DNA, 45 bp, Homo sapiens
  source 1..45: mol_type = genomic DNA
tgtgtggtgaaaacggcgggaggaggaagctacatacctacattt

SEQ ID NO: 387 - DNA, 45 bp, Homo sapiens
  source 1..45: mol_type = genomic DNA
tgtgtggtgtaccatgcatcaggaggaagctacatacctacattt

SEQ ID NO: 388 - DNA, 45 bp, Homo sapiens
  source 1..45: mol_type = genomic DNA
tgtgtggtgaacggagcatcaggaggaagctacatacctacattt

SEQ ID NO: 389 - DNA, 45 bp, Homo sapiens
  source 1..45: mol_type = genomic DNA
tgtgtggtgaggcttgcatcaggaggaagctacatacctacattt

SEQ ID NO: 390 - DNA, 45 bp, Homo sapiens
  source 1..45: mol_type = genomic DNA
tgtgtggtgaccccagcatcaggaggaagctacatacctacattt

SEQ ID NO: 391 - DNA, 45 bp, Homo sapiens
  source 1..45: mol_type = genomic DNA
tgtgtggtgaacatagcaggaggaggaagctacatacctacattt

SEQ ID NO: 392 - DNA, 45 bp, Homo sapiens
  source 1..45: mol_type = genomic DNA
tgtgtggtgaaccatgcatcaggaggaagctacatacctacattt

SEQ ID NO: 393 - DNA, 45 bp, Homo sapiens
  source 1..45: mol_type = genomic DNA
tgtgtggtgaaatcggcggggggaggaagctacatacctacattt

SEQ ID NO: 394 - DNA, 45 bp, Homo sapiens
  source 1..45: mol_type = genomic DNA
tgtgtggtgaatctagcatcaggaggaagctacatacctacattt

SEQ ID NO: 395 - DNA, 45 bp, Homo sapiens
  source 1..45: mol_type = genomic DNA
tgtgtggtcaaaagcgcatcaggaggaagctacatacctacattt

SEQ ID NO: 396 - DNA, 45 bp, Homo sapiens
  source 1..45: mol_type = genomic DNA
tgtgtggtgaacggggcatcaggaggaagctacatacctacattt

SEQ ID NO: 397 - DNA, 45 bp, Homo sapiens
  source 1..45: mol_type = genomic DNA
tgtgtggtgtcgttcgcatcaggaggaagctacatacctacattt

SEQ ID NO: 398 - DNA, 45 bp, Homo sapiens
  source 1..45: mol_type = genomic DNA
tgtgtggtgaactccgcatcaggaggaagctacatacctacattt

SEQ ID NO: 399 - DNA, 45 bp, Homo sapiens
  source 1..45: mol_type = genomic DNA
tgtgtggttaggcatgcatcaggaggaagctacatacctacattt

SEQ ID NO: 400 - DNA, 45 bp, Homo sapiens
  source 1..45: mol_type = genomic DNA
tgtgtggtgagggccgcatcaggaggaagctacatacctacattt

SEQ ID NO: 401 - DNA, 45 bp, Homo sapiens
  source 1..45: mol_type = genomic DNA
tgtgtggtgaccctcgcatcaggaggaagctacatacctacattt

SEQ ID NO: 402 - DNA, 45 bp, Homo sapiens
  source 1..45: mol_type = genomic DNA
tgtgtggtgagactagcatcaggaggaagctacatacctacattt

SEQ ID NO: 403 - DNA, 45 bp, Homo sapiens
  source 1..45: mol_type = genomic DNA
tgtgtggtgaacggggcgtcaggaggaagctacatacctacattt

SEQ ID NO: 404 - DNA, 45 bp, Homo sapiens
  source 1..45: mol_type = genomic DNA
tgtgtggtgaacatggcggcaggaggaagctacatacctacattt

SEQ ID NO: 405 - DNA, 45 bp, Homo sapiens
  source 1..45: mol_type = genomic DNA
tgtgtggtgaaccaggcggcaggaggaagctacatacctacattt

SEQ ID NO: 406 - DNA, 45 bp, Homo sapiens
  source 1..45: mol_type = genomic DNA
tgtgtggtaccccgtgcatcaggaggaagctacatacctacattt

SEQ ID NO: 407 - DNA, 45 bp, Homo sapiens
  source 1..45: mol_type = genomic DNA
tgtgtggtgaacatagcaacaggaggaagctacatacctacattt

SEQ ID NO: 408 - DNA, 45 bp, Homo sapiens
  source 1..45: mol_type = genomic DNA
tgtgtggttagacatgcatcaggaggaagctacatacctacattt

SEQ ID NO: 409 - DNA, 45 bp, Homo sapiens
  source 1..45: mol_type = genomic DNA
tgtgtggtgagcctagcatcaggaggaagctacatacctacattt

SEQ ID NO: 410 - DNA, 45 bp, Homo sapiens
  source 1..45: mol_type = genomic DNA
tgtgtggtgagagcggcatcaggaggaagctacatacctacattt

SEQ ID NO: 411 - DNA, 45 bp, Homo sapiens
  source 1..45: mol_type = genomic DNA
tgtgtggtgaacctagcatcaggaggaagctacatacctacattt

SEQ ID NO: 412 - DNA, 45 bp, Homo sapiens
  source 1..45: mol_type = genomic DNA
tgtgtggtgaacgtcgcatcaggaggaagctacatacctacattt

SEQ ID NO: 413 - DNA, 45 bp, Homo sapiens
  source 1..45: mol_type = genomic DNA
tgtgtggtgagcctcgcatcaggaggaagctacatacctacattt

SEQ ID NO: 414 - DNA, 45 bp, Homo sapiens
  source 1..45: mol_type = genomic DNA
tgtgtggtgaatacggccgggggaggaagctacatacctacattt

SEQ ID NO: 415 - DNA, 45 bp, Homo sapiens
  source 1..45: mol_type = genomic DNA
tgtgtggtgaacatcgcggcaggaggaagctacatacctacattt

SEQ ID NO: 416 - DNA, 45 bp, Homo sapiens
  source 1..45: mol_type = genomic DNA
tgtgtggtccttacggcatcaggaggaagctacatacctacattt

SEQ ID NO: 417 - DNA, 45 bp, Homo sapiens
  source 1..45: mol_type = genomic DNA
tgtgtggtgtcccatgcatcaggaggaagctacatacctacattt

SEQ ID NO: 418 - DNA, 45 bp, Homo sapiens
  source 1..45: mol_type = genomic DNA
tgtgtggtgctatccgcatcaggaggaagctacatacctacattt

SEQ ID NO: 419 - DNA, 45 bp, Homo sapiens
  source 1..45: mol_type = genomic DNA
tgtgtggtgaccctagcatcaggaggaagctacatacctacattt

SEQ ID NO: 420 - DNA, 45 bp, Homo sapiens
  source 1..45: mol_type = genomic DNA
tgtgtggtgagtcttgcatcaggaggaagctacatacctacattt

SEQ ID NO: 421 - DNA, 45 bp, Homo sapiens
  source 1..45: mol_type = genomic DNA
tgtgtggtgagtggggccgtgggaggaagttacatacctacattt

SEQ ID NO: 422 - DNA, 45 bp, Homo sapiens
  source 1..45: mol_type = genomic DNA
tgtgtggtgagtggggccgtgggaggaagctacatacctacattt

SEQ ID NO: 423 - DNA, 45 bp, Homo sapiens
  source 1..45: mol_type = genomic DNA
tgtgtggtgagccatgcatcaggaggaagctacatacctacattt

SEQ ID NO: 424 - DNA, 45 bp, Homo sapiens
  source 1..45: mol_type = genomic DNA
tgtgtggtgaattcagcatcaggaggaagctacatacctacattt

SEQ ID NO: 425 - DNA, 45 bp, Homo sapiens
  source 1..45: mol_type = genomic DNA
tgtgtggtgaacatcgccgggggaggaagctacatacctacattt

SEQ ID NO: 426 - DNA, 45 bp, Homo sapiens
  source 1..45: mol_type = genomic DNA
tgtgtggtgaacatggcttcaggaggaagctacatacctacattt

SEQ ID NO: 427 - DNA, 45 bp, Homo sapiens
  source 1..45: mol_type = genomic DNA
tgtgtggtgaacatggcagcaggaggaagctacatacctacattt

SEQ ID NO: 428 - DNA, 45 bp, Homo sapiens
  source 1..45: mol_type = genomic DNA
tgtgtggtgacctctgcatcaggaggaagctacatacctacattt

SEQ ID NO: 429 - DNA, 45 bp, Homo sapiens
  source 1..45: mol_type = genomic DNA
tgtgtggtgaacttagccagaggaggaagctacatacctacattt

SEQ ID NO: 430 - DNA, 45 bp, Homo sapiens
  source 1..45: mol_type = genomic DNA
tgtgtggtgaaccccgcatcaggaggaagctacatacctacattt

SEQ ID NO: 431 - DNA, 45 bp, Homo sapiens
  source 1..45: mol_type = genomic DNA
tgtgtggtgaacattgcctcaggaggaagctacatacctacattt

SEQ ID NO: 432 - DNA, 45 bp, Homo sapiens
  source 1..45: mol_type = genomic DNA
tgtgtggtgaactcagcttcaggaggaagctacatacctacattt

SEQ ID NO: 433 - DNA, 45 bp, Homo sapiens
  source 1..45: mol_type = genomic DNA
tgtgtggtcagccaagcttcaggaggaagctacatacctacattt

SEQ ID NO: 434 - DNA, 45 bp, Homo sapiens
  source 1..45: mol_type = genomic DNA
tgtgtggtgaacgaggcatcaggaggaagctacatacctacattt

SEQ ID NO: 435 - DNA, 45 bp, Homo sapiens
  source 1..45: mol_type = genomic DNA
tgtgtggtgagtcatgcatcaggaggaagctacatacctacattt

SEQ ID NO: 436 - DNA, 45 bp, Homo sapiens
  source 1..45: mol_type = genomic DNA
tgtgtggtgagcacggcaacaggaggaagctacatacctacattt

SEQ ID NO: 437 - DNA, 45 bp, Homo sapiens
  source 1..45: mol_type = genomic DNA
tgtgtggtgaacagcgcatcaggaggaagctacatacctacattt

SEQ ID NO: 438 - DNA, 45 bp, Homo sapiens
  source 1..45: mol_type = genomic DNA
tgtgtggtgaacagagcatcaggaggaagctacatacctacattt

SEQ ID NO: 439 - DNA, 45 bp, Homo sapiens
  source 1..45: mol_type = genomic DNA
tgtgtggtgtatcatgcatcaggaggaagctacatacctacattt

SEQ ID NO: 440 - DNA, 45 bp, Homo sapiens
  source 1..45: mol_type = genomic DNA
tgtgtggtgatccaagcatcaggaggaagctacatacctacattt

SEQ ID NO: 441 - DNA, 45 bp, Homo sapiens
  source 1..45: mol_type = genomic DNA
tgtgtggtgatcgatgcatcaggaggaagctacatacctacattt

SEQ ID NO: 442 - DNA, 45 bp, Homo sapiens
  source 1..45: mol_type = genomic DNA
tgtgtggtgaacccagcatcaggaggaagctacatacctacattt

SEQ ID NO: 443 - DNA, 45 bp, Homo sapiens
  source 1..45: mol_type = genomic DNA
tgtgtggtgaacgaagcatcaggaggaagctacatacctacattt

SEQ ID NO: 444 - DNA, 45 bp, Homo sapiens
  source 1..45: mol_type = genomic DNA
tgtgtggtgaacatggcgctcggaggaagctacatacctacattt

SEQ ID NO: 445 - DNA, 45 bp, Homo sapiens
  source 1..45: mol_type = genomic DNA
tgtgtggtgatcccggcatcaggaggaagctacatacctacattt

SEQ ID NO: 446 - DNA, 45 bp, Homo sapiens
  source 1..45: mol_type = genomic DNA
tgtgtggtgacccctgcatcaggaggaagctacatacctacattt

SEQ ID NO: 447 - DNA, 45 bp, Homo sapiens
  source 1..45: mol_type = genomic DNA
tgtgtggtgccccgagcctcaggaggaagctacatacctacattt

SEQ ID NO: 448 - DNA, 45 bp, Homo sapiens
  source 1..45: mol_type = genomic DNA
tgtgtggtttctcatgcatcaggaggaagctacatacctacattt

SEQ ID NO: 449 - DNA, 45 bp, Homo sapiens
  source 1..45: mol_type = genomic DNA
tgtgtggtgtccccggccccaggaggaagctacatacctacattt

SEQ ID NO: 450 - DNA, 45 bp, Homo sapiens
  source 1..45: mol_type = genomic DNA
tgtgtggtgaacgacgcatcaggaggaagctacatacctacattt

SEQ ID NO: 451 - DNA, 45 bp, Homo sapiens
  source 1..45: mol_type = genomic DNA
tgtgtggtgaggaaagcatcaggaggaagctacatacctacattt

SEQ ID NO: 452 - DNA, 45 bp, Homo sapiens
  source 1..45: mol_type = genomic DNA
tgtgtggtgaacactgcatcaggaggaagctacatacctacattt

SEQ ID NO: 453 - DNA, 45 bp, Homo sapiens
  source 1..45: mol_type = genomic DNA
tgtgtggtccgacatgcatcaggaggaagctacatacctacattt

SEQ ID NO: 454 - DNA, 45 bp, Homo sapiens
  source 1..45: mol_type = genomic DNA
tgtgtggtgacccccgcatcaggaggaagctacatacctacattt

SEQ ID NO: 455 - DNA, 45 bp, Homo sapiens
  source 1..45: mol_type = genomic DNA
tgtgtggtgagtattgcatcaggaggaagctacatacctacattt

SEQ ID NO: 456 - DNA, 45 bp, Homo sapiens
  source 1..45: mol_type = genomic DNA
tgtgtggtgagcgttgcatcaggaggaagctacatacctacattt

SEQ ID NO: 457 - DNA, 45 bp, Homo sapiens
  source 1..45: mol_type = genomic DNA
tgtgtggtgaacgcggcgacaggaggaagctacatacctacattt

SEQ ID NO: 458 - DNA, 45 bp, Homo sapiens
  source 1..45: mol_type = genomic DNA
tgtgtggtgaacactgccccaggaggaagctacatacctacattt

SEQ ID NO: 459 - DNA, 45 bp, Homo sapiens
  source 1..45: mol_type = genomic DNA
tgtgtggtgaagaaggcatcaggaggaagctacatacctacattt

SEQ ID NO: 460 - DNA, 45 bp, Homo sapiens
  source 1..45: mol_type = genomic DNA
tgtgtggttcatcatgcatcaggaggaagctacatacctacattt

SEQ ID NO: 461 - DNA, 45 bp, Homo sapiens
  source 1..45: mol_type = genomic DNA
tgtgtggtgagggaagcatcaggaggaagctacatacctacattt

SEQ ID NO: 462 - DNA, 45 bp, Homo sapiens
  source 1..45: mol_type = genomic DNA
tgtgtggtgatccctgcatcaggaggaagctacatacctacattt

SEQ ID NO: 463 - DNA, 45 bp, Homo sapiens
  source 1..45: mol_type = genomic DNA
tgtgtggttgtgatggcctccggaggaagctacatacctacattt

SEQ ID NO: 464 - DNA, 45 bp, Homo sapiens
  source 1..45: mol_type = genomic DNA
tgtgtggtgaacgctgcatcaggaggaagctacatacctacattt

SEQ ID NO: 465 - DNA, 45 bp, Homo sapiens
  source 1..45: mol_type = genomic DNA
tgtgtggtgaaccctgcatcaggaggaagctacatacctacattt

SEQ ID NO: 466 - DNA, 45 bp, Homo sapiens
  source 1..45: mol_type = genomic DNA
tgtgtggtgaacaaagcatcaggaggaagctacatacctacattt

SEQ ID NO: 467 - DNA, 45 bp, Homo sapiens
  source 1..45: mol_type = genomic DNA
tgtgtggtgaacatggcctcaggaggaagctacatacctacattt

SEQ ID NO: 468 - DNA, 45 bp, Homo sapiens
  source 1..45: mol_type = genomic DNA
tgtgtggtgaacatcgcgctaggaggaagctacatacctacattt

SEQ ID NO: 469 - DNA, 45 bp, Homo sapiens
  source 1..45: mol_type = genomic DNA
tgtgtggtgaacgtagcaacaggaggaagctacatacctacattt

SEQ ID NO: 470 - DNA, 45 bp, Homo sapiens
  source 1..45: mol_type = genomic DNA
tgtgtggtgcaccaggcatcaggaggaagctacatacctacattt

SEQ ID NO: 471 - DNA, 45 bp, Homo sapiens
  source 1..45: mol_type = genomic DNA
tgtgtggtgaacctggctgggggaggaagctacatacctacattt

SEQ ID NO: 472 - DNA, 45 bp, Homo sapiens
  source 1..45: mol_type = genomic DNA
tgtgtggttcgacatgcatcaggaggaagctacatacctacattt

SEQ ID NO: 473 - DNA, 45 bp, Homo sapiens
  source 1..45: mol_type = genomic DNA
tgtgtggtgaaccgtgcatcaggaggaagctacatacctacattt

SEQ ID NO: 474 - DNA, 45 bp, Homo sapiens
  source 1..45: mol_type = genomic DNA
tgtgtggtccaccatgcatcaggaggaagctacatacctacattt

SEQ ID NO: 475 - DNA, 45 bp, Homo sapiens
  source 1..45: mol_type = genomic DNA
tgtgtggtgagaactgcatcaggaggaagctacatacctacattt

SEQ ID NO: 476 - DNA, 45 bp, Homo sapiens
  source 1..45: mol_type = genomic DNA
tgtgtggtgaactcggcatcaggaggaagctacatacctacattt

SEQ ID NO: 477 - DNA, 45 bp, Homo sapiens
  source 1..45: mol_type = genomic DNA
tgtgtggttcgtcttgcatcaggaggaagctacatacctacattt

SEQ ID NO: 478 - DNA, 45 bp, Homo sapiens
  source 1..45: mol_type = genomic DNA
tgtgtggttagccatgcatcaggaggaagctacatacctacattt

SEQ ID NO: 479 - DNA, 45 bp, Homo sapiens
  source 1..45: mol_type = genomic DNA
tgtgtggtgtcacatgcatcaggaggaagctacatacctacattt

SEQ ID NO: 480 - DNA, 45 bp, Homo sapiens
  source 1..45: mol_type = genomic DNA
tgtgtggtgctcaccgcatcaggaggaagctacatacctacattt

SEQ ID NO: 481 - DNA, 45 bp, Homo sapiens
  source 1..45: mol_type = genomic DNA
tgtgtggtgccccatgcatcaggaggaagctacatacctacattt

SEQ ID NO: 482 - DNA, 45 bp, Homo sapiens
  source 1..45: mol_type = genomic DNA
tgtgtggtgatcgcggcatcaggaggaagctacatacctacattt

SEQ ID NO: 483 - DNA, 45 bp, Homo sapiens
  source 1..45: mol_type = genomic DNA
tgtgtggtgagggtagcggcaggaggaagctacatacctacattt

SEQ ID NO: 484 - DNA, 45 bp, Homo sapiens
  source 1..45: mol_type = genomic DNA
tgtgtggtgagagaggcatcaggaggaagctacatacctacattt

SEQ ID NO: 485 - DNA, 45 bp, Homo sapiens
  source 1..45: mol_type = genomic DNA
tgtgtggtgaaatccgcatcaggaggaagctacatacctacattt

SEQ ID NO: 486 - DNA, 45 bp, Homo sapiens
  source 1..45: mol_type = genomic DNA
tgtgtggtgaattctgcatcaggaggaagctacatacctacattt

SEQ ID NO: 487 - DNA, 45 bp, Homo sapiens
  source 1..45: mol_type = genomic DNA
tgtgtggtgaacccggcatcaggaggaagctacatacctacattt

SEQ ID NO: 488 - DNA, 45 bp, Homo sapiens
  source 1..45: mol_type = genomic DNA
tgtgtggtgcttagcgcatcaggaggaagctacatacctacattt

SEQ ID NO: 489 - DNA, 45 bp, Homo sapiens
  source 1..45: mol_type = genomic DNA
tgtgtggtgagcgccgcatcaggaggaagctacatacctacattt

SEQ ID NO: 490 - DNA, 45 bp, Homo sapiens
  source 1..45: mol_type = genomic DNA
tgtgtggtgaacatagctgcaggaggaagctacatacctacattt

SEQ ID NO: 491 - DNA, 45 bp, Homo sapiens
  source 1..45: mol_type = genomic DNA
tgtgtggtgggggtcgcatcaggaggaagctacatacctacattt

SEQ ID NO: 492 - DNA, 45 bp, Homo sapiens
  source 1..45: mol_type = genomic DNA
tgtgtggtgagcaccgcatcaggaggaagctacatacctacattt

SEQ ID NO: 493 - DNA, 45 bp, Homo sapiens
  source 1..45: mol_type = genomic DNA
tgtgtggtgcgtcatgcatcaggaggaagctacatacctacattt

SEQ ID NO: 494 - DNA, 45 bp, Homo sapiens
  source 1..45: mol_type = genomic DNA
tgtgtggtgaccattgcggagggaggaagctacatacctacattt

SEQ ID NO: 495 - DNA, 45 bp, Homo sapiens
  source 1..45: mol_type = genomic DNA
tgtgtggtgagcgcggcatcaggaggaagctacatacctacattt

SEQ ID NO: 496 - DNA, 45 bp, Homo sapiens
  source 1..45: mol_type = genomic DNA
tgtgtggtgaggattgcatcaggaggaagctacatacctacattt

SEQ ID NO: 497 - DNA, 45 bp, Homo sapiens
  source 1..45: mol_type = genomic DNA
tgtgtggtgaaccgggcaacaggaggaagctacatacctacattt

SEQ ID NO: 498 - DNA, 45 bp, Homo sapiens
  source 1..45: mol_type = genomic DNA
tgtgtggtgaacgaggcggcaggaggaagctacatacctacattt

SEQ ID NO: 499 - DNA, 45 bp, Homo sapiens
  source 1..45: mol_type = genomic DNA
tgtgtggtgaacatagcggcaggaggaagctacatacctacattt

SEQ ID NO: 500 - DNA, 45 bp, Homo sapiens
  source 1..45: mol_type = genomic DNA
tgtgtggtgaacacggctaaaggaggaagctacatacctacattt

SEQ ID NO: 501 - DNA, 45 bp, Homo sapiens
  source 1..45: mol_type = genomic DNA
tgtgtggtgagtgcggcatcaggaggaagctacatacctacattt

SEQ ID NO: 502 - DNA, 45 bp, Homo sapiens
  source 1..45: mol_type = genomic DNA
tgtgtggttcgtcatgcatcaggaggaagctacatacctacattt

SEQ ID NO: 503 - DNA, 45 bp, Homo sapiens
  source 1..45: mol_type = genomic DNA
tgtgtggtgtcttttgcatcaggaggaagctacatacctacattt

SEQ ID NO: 504 - DNA, 45 bp, Homo sapiens
  source 1..45: mol_type = genomic DNA
tgtgtggtgaccattgcatcaggaggaagctacatacctacattt

SEQ ID NO: 505 - DNA, 45 bp, Homo sapiens
  source 1..45: mol_type = genomic DNA
tgtgtggtgaggggcgcatcaggaggaagctacatacctacattt

SEQ ID NO: 506 - DNA, 45 bp, Homo sapiens
  source 1..45: mol_type = genomic DNA
tgtgtggtgaacgtcgcgtcaggaggaagctacatacctacattt

SEQ ID NO: 507 - DNA, 45 bp, Homo sapiens
  source 1..45: mol_type = genomic DNA
tgtgtggtgaagggagcatcaggaggaagctacatacctacattt

SEQ ID NO: 508 - DNA, 45 bp, Homo sapiens
  source 1..45: mol_type = genomic DNA
tgtgtggtgaacggcgcgccaggaggaagctacatacctacattt

SEQ ID NO: 509 - DNA, 45 bp, Homo sapiens
  source 1..45: mol_type = genomic DNA
tgtgtggtgttccgagcatcaggaggaagctacatacctacattt

SEQ ID NO: 510 - DNA, 45 bp, Homo sapiens
  source 1..45: mol_type = genomic DNA
tgtgtggtgtctcttgcatcaggaggaagctacatacctacattt

SEQ ID NO: 511 - DNA, 45 bp, Homo sapiens
  source 1..45: mol_type = genomic DNA
tgtgtggtgacgctcgcatcaggaggaagctacatacctacattt

SEQ ID NO: 512 - DNA, 45 bp, Homo sapiens
  source 1..45: mol_type = genomic DNA
tgtgtggtgaacatagcagcaggaggaagctacatacctacattt

SEQ ID NO: 513 - DNA, 45 bp, Homo sapiens
  source 1..45: mol_type = genomic DNA
tgtgtggtctctatagcatcaggaggaagctacatacctacattt

SEQ ID NO: 514 - DNA, 45 bp, Homo sapiens
  source 1..45: mol_type = genomic DNA
tgtgtggtgttccatgcatcaggaggaagctacatacctacattt

SEQ ID NO: 515 - DNA, 45 bp, Homo sapiens
  source 1..45: mol_type = genomic DNA
tgtgtggtgaagagggctggaggaggaagctacatacctacattt

SEQ ID NO: 516 - DNA, 45 bp, Homo sapiens
  source 1..45: mol_type = genomic DNA
tgtgtggtgagccgcgcatcaggaggaagctacatacctacattt

SEQ ID NO: 517 - DNA, 45 bp, Homo sapiens
  source 1..45: mol_type = genomic DNA
tgtgtggtgaacatcgcgacaggaggaagctacatacctacattt

SEQ ID NO: 518 - DNA, 45 bp, Homo sapiens
  source 1..45: mol_type = genomic DNA
tgtgtggtgatccccgcatcaggaggaagctacatacctacattt

SEQ ID NO: 519 - DNA, 45 bp, Homo sapiens
  source 1..45: mol_type = genomic DNA
tgtgtggtgaactcagcatcaggaggaagctacatacctacattt

SEQ ID NO: 520 - DNA, 45 bp, Homo sapiens
  source 1..45: mol_type = genomic DNA
tgtgtggtgaactacgcatcaggaggaagctacatacctacattt

SEQ ID NO: 521 - DNA, 45 bp, Homo sapiens
  source 1..45: mol_type = genomic DNA
tgtgtggtgaacggggcgacaggaggaagctacatacctacattt

SEQ ID NO: 522 - DNA, 45 bp, Homo sapiens
  source 1..45: mol_type = genomic DNA
tgtgtggtgaacaaggcatcaggaggaagctacatacctacattt

SEQ ID NO: 523 - DNA, 45 bp, Homo sapiens
  source 1..45: mol_type = genomic DNA
tgtgtggtgaagcatgcatcaggaggaagctacatacctacattt

SEQ ID NO: 524 - DNA, 45 bp, Homo sapiens
  source 1..45: mol_type = genomic DNA
tgtgtggtgaaacccgcatcaggaggaagctacatacctacattt

SEQ ID NO: 525 - DNA, 45 bp, Homo sapiens
  source 1..45: mol_type = genomic DNA
tgtgtggtgatttccgcatcaggaggaagctacatacctacattt

SEQ ID NO: 526 - DNA, 45 bp, Homo sapiens
  source 1..45: mol_type = genomic DNA
tgtgtggtgatccgtgcatcaggaggaagctacatacctacattt

SEQ ID NO: 527 - DNA, 45 bp, Homo sapiens
  source 1..45: mol_type = genomic DNA
tgtgtggtgaccgctgcatcaggaggaagctacatacctacattt

SEQ ID NO: 528 - DNA, 45 bp, Homo sapiens
  source 1..45: mol_type = genomic DNA
tgtgtggtgaaaggggcatcaggaggaagctacatacctacattt

SEQ ID NO: 529 - DNA, 45 bp, Homo sapiens
  source 1..45: mol_type = genomic DNA
tgtgtggtcttccttgcatcaggaggaagctacatacctacattt

SEQ ID NO: 530 - DNA, 45 bp, Homo sapiens
  source 1..45: mol_type = genomic DNA
tgtgtggtgaaccaagcatcaggaggaagctacatacctacattt

SEQ ID NO: 531 - DNA, 45 bp, Homo sapiens
  source 1..45: mol_type = genomic DNA
tgtgtggtgaacattgcaacaggaggaagctacatacctacattt

SEQ ID NO: 532 - DNA, 45 bp, Homo sapiens
  source 1..45: mol_type = genomic DNA
tgtgtggtgtccttagcatcaggaggaagctacatacctacattt

SEQ ID NO: 533 - DNA, 45 bp, Homo sapiens
  source 1..45: mol_type = genomic DNA
tgtgtggtgcggcttgcatcaggaggaagctacatacctacattt

SEQ ID NO: 534 - DNA, 45 bp, Homo sapiens
  source 1..45: mol_type = genomic DNA
tgtgtggtccgccccgcgtcaggaggaagctacatacctacattt

SEQ ID NO: 535 - DNA, 45 bp, Homo sapiens
  source 1..45: mol_type = genomic DNA
tgtgtggtgagtgttgcatcaggaggaagctacatacctacattt

SEQ ID NO: 536 - DNA, 45 bp, Homo sapiens
  source 1..45: mol_type = genomic DNA
tgtgtggtgttttccgcatcaggaggaagctacatacctacattt

SEQ ID NO: 537 - DNA, 45 bp, Homo sapiens
  source 1..45: mol_type = genomic DNA
tgtgtggtgaccccggcaccaggaggaagctacatacctacattt

SEQ ID NO: 538 - DNA, 45 bp, Homo sapiens
  source 1..45: mol_type = genomic DNA
tgtgtggtgagtaccgcatcaggaggaagctacatacctacattt

SEQ ID NO: 539 - DNA, 45 bp, Homo sapiens
  source 1..45: mol_type = genomic DNA
tgtgtggtgattccagcatcaggaggaagctacatacctacattt

SEQ ID NO: 540 - DNA, 45 bp, Homo sapiens
  source 1..45: mol_type = genomic DNA
tgtgtggtgaatcatgcatcaggaggaagctacatacctacattt

SEQ ID NO: 541 - DNA, 45 bp, Homo sapiens
  source 1..45: mol_type = genomic DNA
tgtgtggtgaaccaggcatcaggaggaagctacatacctacattt

SEQ ID NO: 542 - DNA, 45 bp, Homo sapiens
  source 1..45: mol_type = genomic DNA
tgtgtggtgaacgcggcatcaggaggaagctacatacctacattt

SEQ ID NO: 543 - DNA, 45 bp, Homo sapiens
  source 1..45: mol_type = genomic DNA
tgtgtggtgaacggagcgtcaggaggaagctacatacctacattt

SEQ ID NO: 544 - DNA, 45 bp, Homo sapiens
  source 1..45: mol_type = genomic DNA
tgtgtggtgaagttggcatcaggaggaagctacatacctacattt

SEQ ID NO: 545 - DNA, 45 bp, Homo sapiens
  source 1..45: mol_type = genomic DNA
tgtgtggtggctactgcatcaggaggaagctacatacctacattt

SEQ ID NO: 546 - DNA, 45 bp, Homo sapiens
  source 1..45: mol_type = genomic DNA
tgtgtggtgatgcccgcatcaggaggaagctacatacctacattt

SEQ ID NO: 547 - DNA, 45 bp, Homo sapiens
  source 1..45: mol_type = genomic DNA
tgtgtggtctccttagcatcaggaggaagctacatacctacattt

SEQ ID NO: 548 - DNA, 45 bp, Homo sapiens
  source 1..45: mol_type = genomic DNA
tgtgtggtgagggaggcatcaggaggaagctacatacctacattt

SEQ ID NO: 549 - DNA, 45 bp, Homo sapiens
  source 1..45: mol_type = genomic DNA
tgtgtggtgaacattgcagggggaggaagctacatacctacattt

SEQ ID NO: 550 - DNA, 45 bp, Homo sapiens
  source 1..45: mol_type = genomic DNA
tgtgtggtgcgcctcgcatcaggaggaagctacatacctacattt

SEQ ID NO: 551 - DNA, 45 bp, Homo sapiens
  source 1..45: mol_type = genomic DNA
tgtgtggtgcgcaccgcatcaggaggaagctacatacctacattt

SEQ ID NO: 552 - DNA, 45 bp, Homo sapiens
  source 1..45: mol_type = genomic DNA
tgtgtggtgagcattgcatcaggaggaagctacatacctacattt

SEQ ID NO: 553 - DNA, 45 bp, Homo sapiens
  source 1..45: mol_type = genomic DNA
tgtgtggtgagacgagcatcaggaggaagctacatacctacattt

SEQ ID NO: 554 - DNA, 45 bp, Homo sapiens
  source 1..45: mol_type = genomic DNA
tgtgtggtttctatcgcatcaggaggaagctacatacctacattt

SEQ ID NO: 555 - DNA, 45 bp, Homo sapiens
  source 1..45: mol_type = genomic DNA
tgtgtggttaagaaggcatcaggaggaagctacatacctacattt

SEQ ID NO: 556 - DNA, 45 bp, Homo sapiens
  source 1..45: mol_type = genomic DNA
tgtgtggtgaggttggcatcaggaggaagctacatacctacattt

SEQ ID NO: 557 - DNA, 45 bp, Homo sapiens
  source 1..45: mol_type = genomic DNA
tgtgtggtgcgcttggcatcaggaggaagctacatacctacattt

SEQ ID NO: 558 - DNA, 45 bp, Homo sapiens
  source 1..45: mol_type = genomic DNA
tgtgtggtgcgagaggcatcaggaggaagctacatacctacattt

SEQ ID NO: 559 - DNA, 45 bp, Homo sapiens
  source 1..45: mol_type = genomic DNA
tgtgtggtgagtctcgcatcaggaggaagctacatacctacattt

SEQ ID NO: 560 - DNA, 45 bp, Homo sapiens
  source 1..45: mol_type = genomic DNA
tgtgtggtctttgctgcatcaggaggaagctacatacctacattt

SEQ ID NO: 561 - DNA, 45 bp, Homo sapiens
  source 1..45: mol_type = genomic DNA
tgtgtggtgttcctcgcggggggaggaagctacatacctacattt

SEQ ID NO: 562 - DNA, 45 bp, Homo sapiens
  source 1..45: mol_type = genomic DNA
tgtgtggtgtccaaggcatcaggaggaagctacatacctacattt

SEQ ID NO: 563 - DNA, 45 bp, Homo sapiens
  source 1..45: mol_type = genomic DNA
tgtgtggtgtcgcttgcatcaggaggaagctacatacctacattt

SEQ ID NO: 564 - DNA, 45 bp, Homo sapiens
  source 1..45: mol_type = genomic DNA
tgtgtggtgagactggcttcaggaggaagctacatacctacattt

SEQ ID NO: 565 - DNA, 45 bp, Homo sapiens
  source 1..45: mol_type = genomic DNA
tgtgtggtggctcaagcatcaggaggaagctacatacctacattt

SEQ ID NO: 566 - DNA, 45 bp, Homo sapiens
  source 1..45: mol_type = genomic DNA
tgtgtggtgaagagggcctccggaggaagctacatacctacattt

SEQ ID NO: 567 - DNA, 45 bp, Homo sapiens
  source 1..45: mol_type = genomic DNA
tgtgtggtgttctcggcatcaggaggaagctacatacctacattt

SEQ ID NO: 568 - DNA, 45 bp, Homo sapiens
  source 1..45: mol_type = genomic DNA
tgtgtggtgagctccgcatcaggaggaagctacatacctacattt

SEQ ID NO: 569 - DNA, 45 bp, Homo sapiens
  source 1..45: mol_type = genomic DNA
tgtgtggtgcctcatgcatcaggaggaagctacatacctacattt

SEQ ID NO: 570 - DNA, 45 bp, Homo sapiens
  source 1..45: mol_type = genomic DNA
tgtgtggtgagcaaggcatcaggaggaagctacatacctacattt

SEQ ID NO: 571 - DNA, 45 bp, Homo sapiens
  source 1..45: mol_type = genomic DNA
tgtgtggtgaacagggcgacaggaggaagctacatacctacattt

SEQ ID NO: 572 - DNA, 45 bp, Homo sapiens
  source 1..45: mol_type = genomic DNA
tgtgtggtgaagggtgcatcaggaggaagctacatacctacattt

SEQ ID NO: 573 - DNA, 45 bp, Homo sapiens
  source 1..45: mol_type = genomic DNA
tgtgtggtgcgccatgcatcaggaggaagctacatacctacattt

SEQ ID NO: 574 - DNA, 45 bp, Homo sapiens
  source 1..45: mol_type = genomic DNA
tgtgtggtgaccgccgcctcaggaggaagctacatacctacattt

SEQ ID NO: 575 - DNA, 45 bp, Homo sapiens
  source 1..45: mol_type = genomic DNA
tgtgtggtgagcgtcgcatcaggaggaagctacatacctacattt

SEQ ID NO: 576 - DNA, 45 bp, Homo sapiens
  source 1..45: mol_type = genomic DNA
tgtgtggtgaacatggcccaaggaggaagctacatacctacattt

SEQ ID NO: 577 - DNA, 45 bp, Homo sapiens
  source 1..45: mol_type = genomic DNA
tgtgtggtgaacatagccatcggaggaagctacatacctacattt

SEQ ID NO: 578 - DNA, 45 bp, Homo sapiens
  source 1..45: mol_type = genomic DNA
tgtgtggtgaacatagcgtcaggaggaagctacatacctacattt

SEQ ID NO: 579 - DNA, 45 bp, Homo sapiens
  source 1..45: mol_type = genomic DNA
tgtgtggtgtccgtcgcatcaggaggaagctacatacctacattt

SEQ ID NO: 580 - DNA, 45 bp, Homo sapiens
  source 1..45: mol_type = genomic DNA
tgtgtggtgagttctgcatcaggaggaagctacatacctacattt

SEQ ID NO: 581 - DNA, 45 bp, Homo sapiens
  source 1..45: mol_type = genomic DNA
tgtgtggtgtcccttgcatcaggaggaagctacatacctacattt

SEQ ID NO: 582 - DNA, 45 bp, Homo sapiens
  source 1..45: mol_type = genomic DNA
tgtgtggtgaacaaagcctcaggaggaagctacatacctacattt

SEQ ID NO: 583 - DNA, 45 bp, Homo sapiens
  source 1..45: mol_type = genomic DNA
tgtgtggtgatcatcgcatcaggaggaagctacatacctacattt

SEQ ID NO: 584 - DNA, 45 bp, Homo sapiens
  source 1..45: mol_type = genomic DNA
tgtgtggtgaacggggcgccaggaggaagctacatacctacattt

SEQ ID NO: 585 - DNA, 45 bp, Homo sapiens
  source 1..45: mol_type = genomic DNA
tgtgtggtgaaaacggcggcaggaggaagctacatacctacattt

SEQ ID NO: 586 - DNA, 45 bp, Homo sapiens
  source 1..45: mol_type = genomic DNA
tgtgtggtccgcgtagcatcaggaggaagctacatacctacattt

SEQ ID NO: 587 - DNA, 45 bp, Homo sapiens
  source 1..45: mol_type = genomic DNA
tgtgtggtgagatcggcatcaggaggaagctacatacctacattt

SEQ ID NO: 588 - DNA, 45 bp, Homo sapiens
  source 1..45: mol_type = genomic DNA
tgtgtggtgaacatggcgtcaggaggaagctacatacctacattt

SEQ ID NO: 589 - DNA, 45 bp, Homo sapiens
  source 1..45: mol_type = genomic DNA
tgtgtggtgagaatcgcatcaggaggaagctacatacctacattt

SEQ ID NO: 590 - DNA, 45 bp, Homo sapiens
  source 1..45: mol_type = genomic DNA
tgtgtggtgaccttggcctcaggaggaagctacatacctacattt

SEQ ID NO: 591 - DNA, 45 bp, Homo sapiens
  source 1..45: mol_type = genomic DNA
tgtgtggtggcgtcagcatcaggaggaagctacatacctacattt

SEQ ID NO: 592 - DNA, 45 bp, Homo sapiens
  source 1..45: mol_type = genomic DNA
tgtgtggtgaacatcgcgtcaggaggaagctacatacctacattt

SEQ ID NO: 593 - DNA, 45 bp, Homo sapiens
  source 1..45: mol_type = genomic DNA
tgtgtggtgtcgctcgcatcaggaggaagctacatacctacattt

SEQ ID NO: 594 - DNA, 45 bp, Homo sapiens
  source 1..45: mol_type = genomic DNA
tgtgtggtgagcctggccgggggaggaagctacatacctacattt

SEQ ID NO: 595 - DNA, 45 bp, Homo sapiens
  source 1..45: mol_type = genomic DNA
tgtgtggtgaaccgagcatcaggaggaagctacatacctacattt

SEQ ID NO: 596 - DNA, 45 bp, Homo sapiens
  source 1..45: mol_type = genomic DNA
tgtgtggtgaatagtgcatcaggaggaagctacatacctacattt

SEQ ID NO: 597 - DNA, 45 bp, Homo sapiens
  source 1..45: mol_type = genomic DNA
tgtgtggtgaacggcgcggcaggaggaagctacatacctacattt

SEQ ID NO: 598 - DNA, 45 bp, Homo sapiens
  source 1..45: mol_type = genomic DNA
tgtgtggtgaacttagcatcaggaggaagctacatacctacattt

SEQ ID NO: 599 - DNA, 45 bp, Homo sapiens
  source 1..45: mol_type = genomic DNA
tgtgtggtggccctcgcatcaggaggaagctacatacctacattt

SEQ ID NO: 600 - DNA, 45 bp, Homo sapiens
  source 1..45: mol_type = genomic DNA
tgtgtggtcaagatcgcatcaggaggaagctacatacctacattt

SEQ ID NO: 601 - DNA, 45 bp, Homo sapiens
  source 1..45: mol_type = genomic DNA
tgtgtggtgtcgggggcgtcaggaggaagctacatacctacattt

SEQ ID NO: 602 - DNA, 45 bp, Homo sapiens
  source 1..45: mol_type = genomic DNA
tgtgtggtgactactgcatcaggaggaagctacatacctacattt

SEQ ID NO: 603 - DNA, 45 bp, Homo sapiens
  source 1..45: mol_type = genomic DNA
tgtgtggtgagcacagcatcaggaggaagctacatacctacattt

SEQ ID NO: 604 - DNA, 45 bp, Homo sapiens
  source 1..45: mol_type = genomic DNA
tgtgtggtgagacaggccgggggaggaagctacatacctacattt

SEQ ID NO: 605 - DNA, 45 bp, Homo sapiens
  source 1..45: mol_type = genomic DNA
tgtgtggtgaacctcgcccgcggaggaagctacatacctacattt

SEQ ID NO: 606 - DNA, 45 bp, Homo sapiens
  source 1..45: mol_type = genomic DNA
tgtgtggtgaacatggcaacaggaggaagctacatacctacattt

SEQ ID NO: 607 - DNA, 45 bp, Homo sapiens
  source 1..45: mol_type = genomic DNA
tgtgtggtgtctcgagcatcaggaggaagctacatacctacattt

SEQ ID NO: 608 - DNA, 45 bp, Homo sapiens
  source 1..45: mol_type = genomic DNA
tgtgtggtgaatcgtgcatcaggaggaagctacatacctacattt

SEQ ID NO: 609 - DNA, 45 bp, Homo sapiens
  source 1..45: mol_type = genomic DNA
tgtgtggtgaacatcgcctcaggaggaagctacatacctacattt

SEQ ID NO: 610 - DNA, 45 bp, Homo sapiens
  source 1..45: mol_type = genomic DNA
tgtgtggtttctcgagcatcaggaggaagctacatacctacattt

SEQ ID NO: 611 - DNA, 45 bp, Homo sapiens
  source 1..45: mol_type = genomic DNA
tgtgtggtgtcgcaggcggcaggaggaagctacatacctacattt

SEQ ID NO: 612 - DNA, 45 bp, Homo sapiens
  source 1..45: mol_type = genomic DNA
tgtgtggtgcttctagcatcaggaggaagctacatacctacattt

SEQ ID NO: 613 - DNA, 45 bp, Homo sapiens
  source 1..45: mol_type = genomic DNA
tgtgtggtgatcgccgcatcaggaggaagctacatacctacattt

SEQ ID NO: 614 - DNA, 45 bp, Homo sapiens
  source 1..45: mol_type = genomic DNA
tgtgtggtgaccatcgcatcaggaggaagctacatacctacattt

SEQ ID NO: 615 - DNA, 45 bp, Homo sapiens
  source 1..45: mol_type = genomic DNA
tgtgtggtgagcccggcatcaggaggaagctacatacctacattt

SEQ ID NO: 616 - DNA, 45 bp, Homo sapiens
  source 1..45: mol_type = genomic DNA
tgtgtggtgcggcatgcatcaggaggaagctacatacctacattt

SEQ ID NO: 617 - DNA, 45 bp, Homo sapiens
  source 1..45: mol_type = genomic DNA
tgtgtggtgaaccgcgcatcaggaggaagctacatacctacattt

SEQ ID NO: 618 - DNA, 45 bp, Homo sapiens
  source 1..45: mol_type = genomic DNA
tgtgtggtgaactgggcatcaggaggaagctacatacctacattt

SEQ ID NO: 619 - DNA, 45 bp, Homo sapiens
  source 1..45: mol_type = genomic DNA
tgtgtggtgggcaccgcatcaggaggaagctacatacctacattt

SEQ ID NO: 620 - DNA, 45 bp, Homo sapiens
  source 1..45: mol_type = genomic DNA
tgtgtggtgatcggcgcatcaggaggaagctacatacctacattt

SEQ ID NO: 621 - DNA, 45 bp, Homo sapiens
  source 1..45: mol_type = genomic DNA
tgtgtggtccgcatggcatcaggaggaagctacatacctacattt

SEQ ID NO: 622 - DNA, 45 bp, Homo sapiens
  source 1..45: mol_type = genomic DNA
tgtgtggtgatttttgcatcaggaggaagctacatacctacattt

SEQ ID NO: 623 - DNA, 45 bp, Homo sapiens
  source 1..45: mol_type = genomic DNA
tgtgtggtgatctccgcatcaggaggaagctacatacctacattt

SEQ ID NO: 624 - DNA, 45 bp, Homo sapiens
  source 1..45: mol_type = genomic DNA
tgtgtggtgacttccgcatcaggaggaagctacatacctacattt

SEQ ID NO: 625 - DNA, 45 bp, Homo sapiens
  source 1..45: mol_type = genomic DNA
tgtgtggtgagccgagcctcaggaggaagctacatacctacattt

SEQ ID NO: 626 - DNA, 45 bp, Homo sapiens
  source 1..45: mol_type = genomic DNA
tgtgtggtgtcgctggcatcaggaggaagctacatacctacattt

SEQ ID NO: 627 - DNA, 45 bp, Homo sapiens
  source 1..45: mol_type = genomic DNA
tgtgtggtgaggcaggcatcaggaggaagctacatacctacattt

SEQ ID NO: 628 - DNA, 45 bp, Homo sapiens
  source 1..45: mol_type = genomic DNA
tgtgtggtccgtctcgcatcaggaggaagctacatacctacattt

SEQ ID NO: 629 - DNA, 45 bp, Homo sapiens
  source 1..45: mol_type = genomic DNA
tgtgtggtgagtggggcatcaggaggaagctacatacctacattt

SEQ ID NO: 630 - DNA, 45 bp, Homo sapiens
  source 1..45: mol_type = genomic DNA
tgtgtggtgaccttcgcatcaggaggaagctacatacctacattt

SEQ ID NO: 631 - DNA, 45 bp, Homo sapiens
  source 1..45: mol_type = genomic DNA
tgtgtggtgagggtcgcttcaggaggaagctacatacctacattt

SEQ ID NO: 632 - DNA, 45 bp, Homo sapiens
  source 1..45: mol_type = genomic DNA
tgtgtggtgcgaacggcatcaggaggaagctacatacctacattt

SEQ ID NO: 633 - DNA, 45 bp, Homo sapiens
  source 1..45: mol_type = genomic DNA
tgtgtggtgaagcgggcatcaggaggaagctacatacctacattt

SEQ ID NO: 634 - DNA, 45 bp, Homo sapiens
  source 1..45: mol_type = genomic DNA
tgtgtggtcttccatgcatcaggaggaagctacatacctacattt

SEQ ID NO: 635 - DNA, 45 bp, Homo sapiens
  source 1..45: mol_type = genomic DNA
tgtgtggtgaaggtcgcatcaggaggaagctacatacctacattt

SEQ ID NO: 636 - DNA, 45 bp, Homo sapiens
  source 1..45: mol_type = genomic DNA
tgtgtggtggggttggcatcaggaggaagctacatacctacattt

SEQ ID NO: 637 - DNA, 45 bp, Homo sapiens
  source 1..45: mol_type = genomic DNA
tgtgtggtgtcctcggcatcaggaggaagctacatacctacattt

SEQ ID NO: 638 - DNA, 45 bp, Homo sapiens
  source 1..45: mol_type = genomic DNA
tgtgtggtgagctctgcatcaggaggaagctacatacctacattt

SEQ ID NO: 639 - DNA, 45 bp, Homo sapiens
  source 1..45: mol_type = genomic DNA
tgtgtggtgaggtttgctcgaggaggaagctacatacctacattt

SEQ ID NO: 640 - DNA, 45 bp, Homo sapiens
  source 1..45: mol_type = genomic DNA
tgtgtggtgagaacggcatcaggaggaagctacatacctacattt

SEQ ID NO: 641 - DNA, 45 bp, Homo sapiens
  source 1..45: mol_type = genomic DNA
tgtgtggtgaattccgcatcaggaggaagctacatacctacattt

SEQ ID NO: 642 - DNA, 45 bp, Homo sapiens
  source 1..45: mol_type = genomic DNA
tgtgtggtgaatcaagcttcaggaggaagctacatacctacattt

SEQ ID NO: 643 - DNA, 45 bp, Homo sapiens
  source 1..45: mol_type = genomic DNA
tgtgtggtgaagatcgcatcaggaggaagctacatacctacattt

SEQ ID NO: 644 - DNA, 45 bp, Homo sapiens
  source 1..45: mol_type = genomic DNA
tgtgtggtcctacatgcatcaggaggaagctacatacctacattt

SEQ ID NO: 645 - DNA, 45 bp, Homo sapiens
  source 1..45: mol_type = genomic DNA
tgtgtggtgagttccgcggtaggaggaagctacatacctacattt

SEQ ID NO: 646 - DNA, 45 bp, Homo sapiens
  source 1..45: mol_type = genomic DNA
tgtgtggtgaagttcgcatcaggaggaagctacatacctacattt

SEQ ID NO: 647 - DNA, 45 bp, Homo sapiens
  source 1..45: mol_type = genomic DNA
tgtgtggtgagtagcgcatcaggaggaagctacatacctacattt

SEQ ID NO: 648 - DNA, 45 bp, Homo sapiens
  source 1..45: mol_type = genomic DNA
tgtgtggtgaagccggcatcaggaggaagctacatacctacattt

SEQ ID NO: 649 - DNA, 45 bp, Homo sapiens
  source 1..45: mol_type = genomic DNA
tgtgtggtccgccgcgcatcaggaggaagctacatacctacattt

SEQ ID NO: 650 - DNA, 45 bp, Homo sapiens
  source 1..45: mol_type = genomic DNA
tgtgtggtctctcttgcatcaggaggaagctacatacctacattt

SEQ ID NO: 651 - DNA, 45 bp, Homo sapiens
  source 1..45: mol_type = genomic DNA
tgtgtggtgagtatagcatcaggaggaagctacatacctacattt

SEQ ID NO: 652 - DNA, 45 bp, Homo sapiens
  source 1..45: mol_type = genomic DNA
tgtgtggtgaatagcgcgggaggaggaagctacatacctacattt

SEQ ID NO: 653 - DNA, 45 bp, Homo sapiens
  source 1..45: mol_type = genomic DNA
tgtgtggtgaacgtggcaggaggaggaagctacatacctacattt

SEQ ID NO: 654 - DNA, 45 bp, Homo sapiens
  source 1..45: mol_type = genomic DNA
tgtgtggtgtctttggcatcaggaggaagctacatacctacattt

SEQ ID NO: 655 - DNA, 45 bp, Homo sapiens
  source 1..45: mol_type = genomic DNA
tgtgtggtgtcttctgcatcaggaggaagctacatacctacattt

SEQ ID NO: 656 - DNA, 45 bp, Homo sapiens
  source 1..45: mol_type = genomic DNA
tgtgtggtgagcactgcatcaggaggaagctacatacctacattt

SEQ ID NO: 657 - DNA, 45 bp, Homo sapiens
  source 1..45: mol_type = genomic DNA
tgtgtggtgtggggggcatcaggaggaagctacatacctacattt

SEQ ID NO: 658 - DNA, 45 bp, Homo sapiens
  source 1..45: mol_type = genomic DNA
tgtgtggtgaccacagccggaggaggaagctacatacctacattt

SEQ ID NO: 659 - DNA, 45 bp, Homo sapiens
  source 1..45: mol_type = genomic DNA
tgtgtggtgaacctcgcggcaggaggaagctacatacctacattt

SEQ ID NO: 660 - DNA, 45 bp, Homo sapiens
  source 1..45: mol_type = genomic DNA
tgtgtggtccgactcgcatcaggaggaagctacatacctacattt

SEQ ID NO: 661 - DNA, 45 bp, Homo sapiens
  source 1..45: mol_type = genomic DNA
tgtgtggtgtccggcgcatcaggaggaagctacatacctacattt

SEQ ID NO: 662 - DNA, 45 bp, Homo sapiens
  source 1..45: mol_type = genomic DNA
tgtgtggtgagttccgcatcaggaggaagctacatacctacattt

SEQ ID NO: 663 - DNA, 45 bp, Homo sapiens
  source 1..45: mol_type = genomic DNA
tgtgtggtgaaccttgcggcaggaggaagctacatacctacattt

SEQ ID NO: 664 - DNA, 45 bp, Homo sapiens
  source 1..45: mol_type = genomic DNA
tgtgtggtgcgtctagcatcaggaggaagctacatacctacattt

SEQ ID NO: 665 - DNA, 45 bp, Homo sapiens
  source 1..45: mol_type = genomic DNA
tgtgtggtgatccatgcatcaggaggaagctacatacctacattt

SEQ ID NO: 666 - DNA, 45 bp, Homo sapiens
  source 1..45: mol_type = genomic DNA
tgtgtggtccatcttgcatcaggaggaagctacatacctacattt

SEQ ID NO: 667 - DNA, 45 bp, Homo sapiens
  source 1..45: mol_type = genomic DNA
tgtgtggtgactccggcatcaggaggaagctacatacctacattt

SEQ ID NO: 668 - DNA, 45 bp, Homo sapiens
  source 1..45: mol_type = genomic DNA
tgtgtggtgttttcggcatcaggaggaagctacatacctacattt

SEQ ID NO: 669 - DNA, 45 bp, Homo sapiens
  source 1..45: mol_type = genomic DNA
tgtgtggtgaacgtagcagggggaggaagctacatacctacattt

SEQ ID NO: 670 - DNA, 45 bp, Homo sapiens
  source 1..45: mol_type = genomic DNA
tgtgtggtgactttggcatcaggaggaagctacatacctacattt

SEQ ID NO: 671 - DNA, 45 bp, Homo sapiens
  source 1..45: mol_type = genomic DNA
tgtgtggtgatccttgcatcaggaggaagctacatacctacattt

SEQ ID NO: 672 - DNA, 45 bp, Homo sapiens
  source 1..45: mol_type = genomic DNA
tgtgtggtgatccccgcgacaggaggaagctacatacctacattt

SEQ ID NO: 673 - DNA, 45 bp, Homo sapiens
  source 1..45: mol_type = genomic DNA
tgtgtggtgaccgttgcatcaggaggaagctacatacctacattt

SEQ ID NO: 674 - DNA, 45 bp, Homo sapiens
  source 1..45: mol_type = genomic DNA
tgtgtggtgaacatggctggaggaggaagctacatacctacattt

SEQ ID NO: 675 - DNA, 45 bp, Homo sapiens
  source 1..45: mol_type = genomic DNA
tgtgtggtgaagacagcatcaggaggaagctacatacctacattt

SEQ ID NO: 676 - DNA, 45 bp, Homo sapiens
  source 1..45: mol_type = genomic DNA
tgtgtggtacacgaggcatcaggaggaagctacatacctacattt

SEQ ID NO: 677 - DNA, 45 bp, Homo sapiens
  source 1..45: mol_type = genomic DNA
tgtgtggtccctcatgcatcaggaggaagctacatacctacattt

SEQ ID NO: 678 - DNA, 45 bp, Homo sapiens
  source 1..45: mol_type = genomic DNA
tgtgtggtgatcattgcatcaggaggaagctacatacctacattt

SEQ ID NO: 679 - DNA, 45 bp, Homo sapiens
  source 1..45: mol_type = genomic DNA
tgtgtggtttggccggcatcaggaggaagctacatacctacattt

SEQ ID NO: 680 - DNA, 45 bp, Homo sapiens
  source 1..45: mol_type = genomic DNA
tgtgtggtgagtcctgcatcaggaggaagctacatacctacattt

SEQ ID NO: 681 - DNA, 45 bp, Homo sapiens
  source 1..45: mol_type = genomic DNA
tgtgtggtgaaccctgcttcaggaggaagctacatacctacattt

SEQ ID NO: 682 - DNA, 45 bp, Homo sapiens
  source 1..45: mol_type = genomic DNA
tgtgtggtgaacggggcttcaggaggaagctacatacctacattt

SEQ ID NO: 683 - DNA, 45 bp, Homo sapiens
  source 1..45: mol_type = genomic DNA
tgtgtggtgatcggtgcatcaggaggaagctacatacctacattt

SEQ ID NO: 684 - DNA, 45 bp, Homo sapiens
  source 1..45: mol_type = genomic DNA
tgtgtggtgagccccgcatcaggaggaagctacatacctacattt

SEQ ID NO: 685 - DNA, 45 bp, Homo sapiens
  source 1..45: mol_type = genomic DNA
tgtgtggtgaacgcagcatcaggaggaagctacatacctacattt

SEQ ID NO: 686 - DNA, 45 bp, Homo sapiens
  source 1..45: mol_type = genomic DNA
tgtgtggtggccttcgcatcaggaggaagctacatacctacattt

SEQ ID NO: 687 - DNA, 45 bp, Homo sapiens
  source 1..45: mol_type = genomic DNA
tgtgtggtgaacaccgcgggaggaggaagctacatacctacattt

SEQ ID NO: 688 - DNA, 45 bp, Homo sapiens
  source 1..45: mol_type = genomic DNA
tgtgtggtccggatagcatcaggaggaagctacatacctacattt

SEQ ID NO: 689 - DNA, 45 bp, Homo sapiens
  source 1..45: mol_type = genomic DNA
tgtgtggtgatggccgcatcaggaggaagctacatacctacattt

SEQ ID NO: 690 - DNA, 45 bp, Homo sapiens
  source 1..45: mol_type = genomic DNA
tgtgtggtgaacaaggccgcgggaggaagctacatacctacattt

SEQ ID NO: 691 - DNA, 45 bp, Homo sapiens
  source 1..45: mol_type = genomic DNA
tgtgtggtgacccatgcatcaggaggaagctacatacctacattt

SEQ ID NO: 692 - DNA, 45 bp, Homo sapiens
  source 1..45: mol_type = genomic DNA
tgtgtggtgaccttagcatcaggaggaagctacatacctacattt

SEQ ID NO: 693 - DNA, 45 bp, Homo sapiens
  source 1..45: mol_type = genomic DNA
tgtgtggtgaacgtggcggcaggaggaagctacatacctacattt

SEQ ID NO: 694 - DNA, 45 bp, Homo sapiens
  source 1..45: mol_type = genomic DNA
tgtgtggtgaggttcgcatcaggaggaagctacatacctacattt

SEQ ID NO: 695 - DNA, 45 bp, Homo sapiens
  source 1..45: mol_type = genomic DNA
tgtgtggtgtcctttgcatcaggaggaagctacatacctacattt

SEQ ID NO: 696 - DNA, 45 bp, Homo sapiens
  source 1..45: mol_type = genomic DNA
tgtgtggtgaggtatgcatcaggaggaagctacatacctacattt

SEQ ID NO: 697 - DNA, 45 bp, Homo sapiens
  source 1..45: mol_type = genomic DNA
tgtgtggtgaggcatgcaacaggaggaagctacatacctacattt

SEQ ID NO: 698 - DNA, 45 bp, Homo sapiens
  source 1..45: mol_type = genomic DNA
tgtgtggtgagggtagcatcaggaggaagctacatacctacattt

SEQ ID NO: 699 - DNA, 45 bp, Homo sapiens
  source 1..45: mol_type = genomic DNA
tgtgtggtgaactcggcagcaggaggaagctacatacctacattt

SEQ ID NO: 700 - DNA, 45 bp, Homo sapiens
  source 1..45: mol_type = genomic DNA
tgtgtggtgaacgtagcagcaggaggaagctacatacctacattt

SEQ ID NO: 701 - DNA, 45 bp, Homo sapiens
  source 1..45: mol_type = genomic DNA
tgtgtggtgaacatggccacaggaggaagctacatacctacattt

SEQ ID NO: 702 - DNA, 45 bp, Homo sapiens
  source 1..45: mol_type = genomic DNA
tgtgtggtacgtcgggcatcaggaggaagctacatacctacattt

SEQ ID NO: 703 - DNA, 45 bp, Homo sapiens
  source 1..45: mol_type = genomic DNA
tgtgtggtcgctacggcatcaggaggaagctacatacctacattt

SEQ ID NO: 704 - DNA, 45 bp, Homo sapiens
  source 1..45: mol_type = genomic DNA
tgtgtggtttcccatgcatcaggaggaagctacatacctacattt

SEQ ID NO: 705 - DNA, 45 bp, Homo sapiens
  source 1..45: mol_type = genomic DNA
tgtgtggtgaacggtgcatcaggaggaagctacatacctacattt

SEQ ID NO: 706 - DNA, 45 bp, Homo sapiens
  source 1..45: mol_type = genomic DNA
tgtgtggtgcggaccgcatcaggaggaagctacatacctacattt

SEQ ID NO: 707 - DNA, 45 bp, Homo sapiens
  source 1..45: mol_type = genomic DNA
tgtgtggtgtcaaaagcatcaggaggaagctacatacctacattt

SEQ ID NO: 708 - DNA, 45 bp, Homo sapiens
  source 1..45: mol_type = genomic DNA
tgtgtggtggccttagcatcaggaggaagctacatacctacattt

SEQ ID NO: 709 - DNA, 45 bp, Homo sapiens
  source 1..45: mol_type = genomic DNA
tgtgtggtgaacatcgccgcaggaggaagctacatacctacattt

SEQ ID NO: 710 - DNA, 45 bp, Homo sapiens
  source 1..45: mol_type = genomic DNA
tgtgtggttcgggaggcatcaggaggaagctacatacctacattt

SEQ ID NO: 711 - DNA, 45 bp, Homo sapiens
  source 1..45: mol_type = genomic DNA
tgtgtggttgctcatgcatcaggaggaagctacatacctacattt

SEQ ID NO: 712 - DNA, 45 bp, Homo sapiens
  source 1..45: mol_type = genomic DNA
tgtgtggtgaaagtagctggtggaggaagctacatacctacattt

SEQ ID NO: 713 - DNA, 45 bp, Homo sapiens
  source 1..45: mol_type = genomic DNA
tgtgtggtgcgtactgcatcaggaggaagctacatacctacattt

SEQ ID NO: 714 - DNA, 45 bp, Homo sapiens
  source 1..45: mol_type = genomic DNA
tgtgtggtgaacctcgcgggaggaggaagctacatacctacattt

SEQ ID NO: 715 - DNA, 45 bp, Homo sapiens
  source 1..45: mol_type = genomic DNA
tgtgtggtgaacatagcgggaggaggaagctacatacctacattt

SEQ ID NO: 716 - DNA, 45 bp, Homo sapiens
  source 1..45: mol_type = genomic DNA
tgtgtggtgagtctggcatcaggaggaagctacatacctacattt

SEQ ID NO: 717 - DNA, 45 bp, Homo sapiens
  source 1..45: mol_type = genomic DNA
tgtgtggtgagagcagcatcaggaggaagctacatacctacattt

SEQ ID NO: 718 - DNA, 45 bp, Homo sapiens
  source 1..45: mol_type = genomic DNA
tgtgtggtgaacaaagccccaggaggaagctacatacctacattt

SEQ ID NO: 719 - DNA, 45 bp, Homo sapiens
  source 1..45: mol_type = genomic DNA
tgtgtggtccctttcgcatcaggaggaagctacatacctacattt

SEQ ID NO: 720 - DNA, 45 bp, Homo sapiens
  source 1..45: mol_type = genomic DNA
tgtgtggtgtccctcgcatcaggaggaagctacatacctacattt

SEQ ID NO: 721 - DNA, 45 bp, Homo sapiens
  source 1..45: mol_type = genomic DNA
tgtgtggtgtcgtctgcatcaggaggaagctacatacctacattt

SEQ ID NO: 722 - DNA, 45 bp, Homo sapiens
  source 1..45: mol_type = genomic DNA
tgtgtggtgccggtagcatcaggaggaagctacatacctacattt

SEQ ID NO: 723 - DNA, 45 bp, Homo sapiens
  source 1..45: mol_type = genomic DNA
tgtgtggtgcgacgcgcatcaggaggaagctacatacctacattt

SEQ ID NO: 724 - DNA, 45 bp, Homo sapiens
  source 1..45: mol_type = genomic DNA
tgtgtggtgagtcaggcatcaggaggaagctacatacctacattt

SEQ ID NO: 725 - DNA, 45 bp, Homo sapiens
  source 1..45: mol_type = genomic DNA
tgtgtggtgagggtcgccggaggaggaagctacatacctacattt

SEQ ID NO: 726 - DNA, 45 bp, Homo sapiens
  source 1..45: mol_type = genomic DNA
tgtgtggtgagggcagcatcaggaggaagctacatacctacattt

SEQ ID NO: 727 - DNA, 45 bp, Homo sapiens
  source 1..45: mol_type = genomic DNA
tgtgtggtgaggactgcatcaggaggaagctacatacctacattt

SEQ ID NO: 728 - DNA, 45 bp, Homo sapiens
  source 1..45: mol_type = genomic DNA
tgtgtggtgagaaccgcccaaggaggaagctacatacctacattt

SEQ ID NO: 729 - DNA, 45 bp, Homo sapiens
  source 1..45: mol_type = genomic DNA
tgtgtggtgaatattgcatcaggaggaagctacatacctacattt

SEQ ID NO: 730 - DNA, 45 bp, Homo sapiens
  source 1..45: mol_type = genomic DNA
tgtgtggtgaacacggcgggaggaggaagctacatacctacattt

SEQ ID NO: 731 - DNA, 45 bp, Homo sapiens
  source 1..45: mol_type = genomic DNA
tgtgtggtggcctccgcatcaggaggaagctacatacctacattt

SEQ ID NO: 732 - DNA, 45 bp, Homo sapiens
  source 1..45: mol_type = genomic DNA
tgtgtggtgaggtcggcatcaggaggaagctacatacctacattt

SEQ ID NO: 733 - DNA, 45 bp, Homo sapiens
  source 1..45: mol_type = genomic DNA
tgtgtggtgaggcgcgcatcaggaggaagctacatacctacattt

SEQ ID NO: 734 - DNA, 45 bp, Homo sapiens
  source 1..45: mol_type = genomic DNA
tgtgtggtccgacaggcatcaggaggaagctacatacctacattt

SEQ ID NO: 735 - DNA, 45 bp, Homo sapiens
  source 1..45: mol_type = genomic DNA
tgtgtggtgggcctcgcatcaggaggaagctacatacctacattt

SEQ ID NO: 736 - DNA, 45 bp, Homo sapiens
  source 1..45: mol_type = genomic DNA
tgtgtggtgaccagcgctacaggaggaagctacatacctacattt

SEQ ID NO: 737 - DNA, 45 bp, Homo sapiens
  source 1..45: mol_type = genomic DNA
tgtgtggtgttcctcgcatcaggaggaagctacatacctacattt

SEQ ID NO: 738 - DNA, 45 bp, Homo sapiens
  source 1..45: mol_type = genomic DNA
tgtgtggtgagtggagcatcaggaggaagctacatacctacattt

SEQ ID NO: 739 - DNA, 45 bp, Homo sapiens
  source 1..45: mol_type = genomic DNA
tgtgtggtgagggtcgcagcaggaggaagctacatacctacattt

SEQ ID NO: 740 - DNA, 45 bp, Homo sapiens
  source 1..45: mol_type = genomic DNA
tgtgtggtgacctcagcatcaggaggaagctacatacctacattt

SEQ ID NO: 741 - DNA, 45 bp, Homo sapiens
  source 1..45: mol_type = genomic DNA
tgtgtggtgaacctggcggcaggaggaagctacatacctacattt

SEQ ID NO: 742 - DNA, 45 bp, Homo sapiens
  source 1..45: mol_type = genomic DNA
tgtgtggtgaacgtggcatcaggaggaagctacatacctacattt

SEQ ID NO: 743 - DNA, 45 bp, Homo sapiens
  source 1..45: mol_type = genomic DNA
tgtgtggtgaagacggcatcaggaggaagctacatacctacattt

SEQ ID NO: 744 - DNA, 45 bp, Homo sapiens
  source 1..45: mol_type = genomic DNA
tgtgtggtgtccttcgcctcaggaggaagctacatacctacattt

SEQ ID NO: 745 - DNA, 45 bp, Homo sapiens
  source 1..45: mol_type = genomic DNA
tgtgtggtgagcggtgcatcaggaggaagctacatacctacattt

SEQ ID NO: 746 - DNA, 45 bp, Homo sapiens
  source 1..45: mol_type = genomic DNA
tgtgtggtgcgcctagcatcaggaggaagctacatacctacattt

SEQ ID NO: 747 - DNA, 45 bp, Homo sapiens
  source 1..45: mol_type = genomic DNA
tgtgtggtgagtctagcatcaggaggaagctacatacctacattt

SEQ ID NO: 748 - DNA, 45 bp, Homo sapiens
  source 1..45: mol_type = genomic DNA
tgtgtggtgaacggggccccaggaggaagctacatacctacattt

SEQ ID NO: 749 - DNA, 45 bp, Homo sapiens
  source 1..45: mol_type = genomic DNA
tgtgtggtgaacggggcccaaggaggaagctacatacctacattt

SEQ ID NO: 750 - DNA, 45 bp, Homo sapiens
  source 1..45: mol_type = genomic DNA
tgtgtggtgaaaatggcatcaggaggaagctacatacctacattt

SEQ ID NO: 751 - DNA, 45 bp, Homo sapiens
  source 1..45: mol_type = genomic DNA
tgtgtggtgaaccccgcgtcaggaggaagctacatacctacattt

SEQ ID NO: 752 - DNA, 45 bp, Homo sapiens
  source 1..45: mol_type = genomic DNA
tgtgtggtgcgccttgcatcaggaggaagctacatacctacattt

SEQ ID NO: 753 - DNA, 45 bp, Homo sapiens
  source 1..45: mol_type = genomic DNA
tgtgtggtacgccatgcatcaggaggaagctacatacctacattt

SEQ ID NO: 754 - DNA, 45 bp, Homo sapiens
  source 1..45: mol_type = genomic DNA
tgtgtggtgttcctagcatcaggaggaagctacatacctacattt

SEQ ID NO: 755 - DNA, 45 bp, Homo sapiens
  source 1..45: mol_type = genomic DNA
tgtgtggtgaacctcgcccgaggaggaagctacatacctacattt

SEQ ID NO: 756 - DNA, 45 bp, Homo sapiens
  source 1..45: mol_type = genomic DNA
tgtgtggtcgctcccgcatcaggaggaagctacatacctacattt

SEQ ID NO: 757 - DNA, 45 bp, Homo sapiens
  source 1..45: mol_type = genomic DNA
tgtgtggtgcttggggccgcaggaggaagctacatacctacattt

SEQ ID NO: 758 - DNA, 45 bp, Homo sapiens
  source 1..45: mol_type = genomic DNA
tgtgtggtgctcggcgcatcaggaggaagctacatacctacattt

SEQ ID NO: 759 - DNA, 45 bp, Homo sapiens
  source 1..45: mol_type = genomic DNA
tgtgtggtgatgggtgcatcaggaggaagctacatacctacattt

SEQ ID NO: 760 - DNA, 45 bp, Homo sapiens
  source 1..45: mol_type = genomic DNA
tgtgtggtgagattagcatcaggaggaagctacatacctacattt

SEQ ID NO: 761 - DNA, 45 bp, Homo sapiens
  source 1..45: mol_type = genomic DNA
tgtgtggttccccatgcatcaggaggaagctacatacctacattt

SEQ ID NO: 762 - DNA, 45 bp, Homo sapiens
  source 1..45: mol_type = genomic DNA
tgtgtggtgaccgatgcatcaggaggaagctacatacctacattt

SEQ ID NO: 763 - DNA, 45 bp, Homo sapiens
  source 1..45: mol_type = genomic DNA
tgtgtggtgaccgacgcatcaggaggaagctacatacctacattt

SEQ ID NO: 764 - DNA, 45 bp, Homo sapiens
  source 1..45: mol_type = genomic DNA
tgtgtggtgaacaaggcgacaggaggaagctacatacctacattt

SEQ ID NO: 765 - DNA, 45 bp, Homo sapiens
  source 1..45: mol_type = genomic DNA
tgtgtggtgaatcgggcatcaggaggaagctacatacctacattt

SEQ ID NO: 766 - DNA, 45 bp, Homo sapiens
  source 1..45: mol_type = genomic DNA
tgtgtggtgaagggggcctcaggaggaagctacatacctacattt

SEQ ID NO: 767 - DNA, 45 bp, Homo sapiens
  source 1..45: mol_type = genomic DNA
tgtgtggtggtaggggcatcaggaggaagctacatacctacattt

SEQ ID NO: 768 - DNA, 45 bp, Homo sapiens
  source 1..45: mol_type = genomic DNA
tgtgtggtggatcatgcatcaggaggaagctacatacctacattt

SEQ ID NO: 769 - DNA, 45 bp, Homo sapiens
  source 1..45: mol_type = genomic DNA
tgtgtggtgttagttgcatcaggaggaagctacatacctacattt

SEQ ID NO: 770 - DNA, 45 bp, Homo sapiens
  source 1..45: mol_type = genomic DNA
tgtgtggtgacccaagcatcaggaggaagctacatacctacattt

SEQ ID NO: 771 - DNA, 45 bp, Homo sapiens
  source 1..45: mol_type = genomic DNA
tgtgtggtgaggatagcatcaggaggaagctacatacctacattt

SEQ ID NO: 772 - DNA, 45 bp, Homo sapiens
  source 1..45: mol_type = genomic DNA
tgtgtggtgactgccgcatcaggaggaagctacatacctacattt

SEQ ID NO: 773 - DNA, 45 bp, Homo sapiens
  source 1..45: mol_type = genomic DNA
tgtgtggtgtcccccgcatcaggaggaagctacatacctacattt

SEQ ID NO: 774 - DNA, 45 bp, Homo sapiens
  source 1..45: mol_type = genomic DNA
tgtgtggtgattcctgcatcaggaggaagctacatacctacattt

SEQ ID NO: 775 - DNA, 45 bp, Homo sapiens
  source 1..45: mol_type = genomic DNA
tgtgtggtgaaggcagcatcaggaggaagctacatacctacattt

SEQ ID NO: 776 - DNA, 45 bp, Homo sapiens
  source 1..45: mol_type = genomic DNA
tgtgtggtgaggcgggcatcaggaggaagctacatacctacattt

SEQ ID NO: 777 - DNA, 45 bp, Homo sapiens
  source 1..45: mol_type = genomic DNA
tgtgtggtgagcctggcatcaggaggaagctacatacctacattt

SEQ ID NO: 778 - DNA, 45 bp, Homo sapiens
  source 1..45: mol_type = genomic DNA
tgtgtggtgaatttggcatcaggaggaagctacatacctacattt

SEQ ID NO: 779 - DNA, 45 bp, Homo sapiens
  source 1..45: mol_type = genomic DNA
tgtgtggtgaacagagcaccaggaggaagctacatacctacattt

SEQ ID NO: 780 - DNA, 45 bp, Homo sapiens
  source 1..45: mol_type = genomic DNA
tgtgtggtgattccggcatcaggaggaagctacatacctacattt

SEQ ID NO: 781 - DNA, 45 bp, Homo sapiens
  source 1..45: mol_type = genomic DNA
tgtgtggtgattaccgcatcaggaggaagctacatacctacattt

SEQ ID NO: 782 - DNA, 45 bp, Homo sapiens
  source 1..45: mol_type = genomic DNA
tgtgtggtgattctagcatcaggaggaagctacatacctacattt

SEQ ID NO: 783 - DNA, 45 bp, Homo sapiens
  source 1..45: mol_type = genomic DNA
tgtgtggtgatctctgcatcaggaggaagctacatacctacattt

SEQ ID NO: 784 - DNA, 45 bp, Homo sapiens
  source 1..45: mol_type = genomic DNA
tgtgtggtgaatcctgcatcaggaggaagctacatacctacattt

SEQ ID NO: 785 - DNA, 45 bp, Homo sapiens
  source 1..45: mol_type = genomic DNA
tgtgtggtgaacttggcttcaggaggaagctacatacctacattt

SEQ ID NO: 786 - DNA, 45 bp, Homo sapiens
  source 1..45: mol_type = genomic DNA
tgtgtggtgaacatcgctgcaggaggaagctacatacctacattt

SEQ ID NO: 787 - DNA, 45 bp, Homo sapiens
  source 1..45: mol_type = genomic DNA
tgtgtggtgaacccagcctcaggaggaagctacatacctacattt

SEQ ID NO: 788 - DNA, 45 bp, Homo sapiens
  source 1..45: mol_type = genomic DNA
tgtgtggtgaaccacgcatcaggaggaagctacatacctacattt

SEQ ID NO: 789 - DNA, 45 bp, Homo sapiens
  source 1..45: mol_type = genomic DNA
tgtgtggtcctgcatgcatcaggaggaagctacatacctacattt

SEQ ID NO: 790 - DNA, 45 bp, Homo sapiens
  source 1..45: mol_type = genomic DNA
tgtgtggtgattctcgcatcaggaggaagctacatacctacattt

SEQ ID NO: 791 - DNA, 45 bp, Homo sapiens
  source 1..45: mol_type = genomic DNA
tgtgtggtgattggggcatcaggaggaagctacatacctacattt

SEQ ID NO: 792 - DNA, 45 bp, Homo sapiens
  source 1..45: mol_type = genomic DNA
tgtgtggtgaacggggcgcgaggaggaagctacatacctacattt

SEQ ID NO: 793 - DNA, 45 bp, Homo sapiens
  source 1..45: mol_type = genomic DNA
tgtgtggtgataggtgcatcaggaggaagctacatacctacattt

SEQ ID NO: 794 - DNA, 45 bp, Homo sapiens
  source 1..45: mol_type = genomic DNA
tgtgtggtgaccggcgcatcaggaggaagctacatacctacattt

SEQ ID NO: 795 - DNA, 45 bp, Homo sapiens
  source 1..45: mol_type = genomic DNA
tgtgtggtgaaccgggcggcaggaggaagctacatacctacattt

SEQ ID NO: 796 - DNA, 45 bp, Homo sapiens
  source 1..45: mol_type = genomic DNA
tgtgtggtgcgtctcgcatcaggaggaagctacatacctacattt

SEQ ID NO: 797 - DNA, 45 bp, Homo sapiens
  source 1..45: mol_type = genomic DNA
tgtgtggtggccgatgcatcaggaggaagctacatacctacattt

SEQ ID NO: 798 - DNA, 45 bp, Homo sapiens
  source 1..45: mol_type = genomic DNA
tgtgtggtgactcatgcatcaggaggaagctacatacctacattt

SEQ ID NO: 799 - DNA, 45 bp, Homo sapiens
  source 1..45: mol_type = genomic DNA
tgtgtggtgaacatcgcaccaggaggaagctacatacctacattt

SEQ ID NO: 800 - DNA, 45 bp, Homo sapiens
  source 1..45: mol_type = genomic DNA
tgtgtggtgaggagcgcatcaggaggaagctacatacctacattt

SEQ ID NO: 801 - DNA, 45 bp, Homo sapiens
  source 1..45: mol_type = genomic DNA
tgtgtggtgatcgctgcatcaggaggaagctacatacctacattt

SEQ ID NO: 802 - DNA, 45 bp, Homo sapiens
  source 1..45: mol_type = genomic DNA
tgtgtggtgaacagcgcggcaggaggaagctacatacctacattt

SEQ ID NO: 803 - DNA, 45 bp, Homo sapiens
  source 1..45: mol_type = genomic DNA
tgtgtggtgccgcatgcatcaggaggaagctacatacctacattt

SEQ ID NO: 804 - DNA, 45 bp, Homo sapiens
  source 1..45: mol_type = genomic DNA
tgtgtggtgaacccggctccaggaggaagctacatacctacattt

SEQ ID NO: 805 - DNA, 45 bp, Homo sapiens
  source 1..45: mol_type = genomic DNA
tgtgtggtgagaggtgcatcaggaggaagctacatacctacattt

SEQ ID NO: 806 - DNA, 45 bp, Homo sapiens
  source 1..45: mol_type = genomic DNA
tgtgtggtgaaggctgcatcaggaggaagctacatacctacattt

SEQ ID NO: 807 - DNA, 45 bp, Homo sapiens
  source 1..45: mol_type = genomic DNA
tgtgtggtgatcctcgcatcaggaggaagctacatacctacattt

SEQ ID NO: 808 - DNA, 45 bp, Homo sapiens
  source 1..45: mol_type = genomic DNA
tgtgtggtgaacggggcggggggaggaagctacatacctacattt

SEQ ID NO: 809 - DNA, 45 bp, Homo sapiens
  source 1..45: mol_type = genomic DNA
tgtgtggtgaacaaggcgaaaggaggaagctacatacctacattt

SEQ ID NO: 810 - DNA, 45 bp, Homo sapiens
  source 1..45: mol_type = genomic DNA
tgtgtggtgaaactggccgtcggaggaagctacatacctacattt

SEQ ID NO: 811 - DNA, 45 bp, Homo sapiens
  source 1..45: mol_type = genomic DNA
tgtgtggtgaaaggcgcatcaggaggaagctacatacctacattt

SEQ ID NO: 812 - DNA, 45 bp, Homo sapiens
  source 1..45: mol_type = genomic DNA
tgtgtggtgaagagggcggggggaggaagctacatacctacattt

SEQ ID NO: 813 - DNA, 45 bp, Homo sapiens
  source 1..45: mol_type = genomic DNA
tgtgtggtgaacgctgccaggggaggaagctacatacctacattt

SEQ ID NO: 814 - DNA, 45 bp, Homo sapiens
  source 1..45: mol_type = genomic DNA
tgtgtggttcattatgcatcaggaggaagctacatacctacattt

SEQ ID NO: 815 - DNA, 45 bp, Homo sapiens
  source 1..45: mol_type = genomic DNA
tgtgtggtggctcatgcatcaggaggaagctacatacctacattt

SEQ ID NO: 816 - DNA, 45 bp, Homo sapiens
  source 1..45: mol_type = genomic DNA
tgtgtggtccagagtgcatcaggaggaagctacatacctacattt

SEQ ID NO: 817 - DNA, 45 bp, Homo sapiens
  source 1..45: mol_type = genomic DNA
tgtgtggtgttccttgcatcaggaggaagctacatacctacattt

SEQ ID NO: 818 - DNA, 45 bp, Homo sapiens
  source 1..45: mol_type = genomic DNA
tgtgtggtgtccttggcatcaggaggaagctacatacctacattt

SEQ ID NO: 819 - DNA, 45 bp, Homo sapiens
  source 1..45: mol_type = genomic DNA
tgtgtggtggtccgagcatcaggaggaagctacatacctacattt

SEQ ID NO: 820 - DNA, 45 bp, Homo sapiens
  source 1..45: mol_type = genomic DNA
tgtgtggtgattgtagcatcaggaggaagctacatacctacattt

SEQ ID NO: 821 - DNA, 45 bp, Homo sapiens
  source 1..45: mol_type = genomic DNA
tgtgtggtgaactctgcgtcaggaggaagctacatacctacattt

SEQ ID NO: 822 - DNA, 45 bp, Homo sapiens
  source 1..45: mol_type = genomic DNA
tgtgtggtgaacggggcagcaggaggaagctacatacctacattt

SEQ ID NO: 823 - DNA, 45 bp, Homo sapiens
  source 1..45: mol_type = genomic DNA
tgtgtggtggggaccgcatcaggaggaagctacatacctacattt

SEQ ID NO: 824 - DNA, 45 bp, Homo sapiens
  source 1..45: mol_type = genomic DNA
tgtgtggtgaacggagccccaggaggaagctacatacctacattt

SEQ ID NO: 825 - DNA, 45 bp, Homo sapiens
  source 1..45: mol_type = genomic DNA
tgtgtggtgattatcgcatcaggaggaagctacatacctacattt

SEQ ID NO: 826 - DNA, 45 bp, Homo sapiens
  source 1..45: mol_type = genomic DNA
tgtgtggtgtctcgggccatgggaggaagctacatacctacattt

SEQ ID NO: 827 - DNA, 45 bp, Homo sapiens
  source 1..45: mol_type = genomic DNA
tgtgtggtgaagaacgcatcaggaggaagctacatacctacattt

SEQ ID NO: 828 - DNA, 45 bp, Homo sapiens
  source 1..45: mol_type = genomic DNA
tgtgtggtgcttcatgcatcaggaggaagctacatacctacattt

SEQ ID NO: 829 - DNA, 45 bp, Homo sapiens
  source 1..45: mol_type = genomic DNA
tgtgtggtgaccgccgcatcaggaggaagctacatacctacattt

SEQ ID NO: 830 - DNA, 45 bp, Homo sapiens
  source 1..45: mol_type = genomic DNA
tgtgtggtgaaacatgcatcaggaggaagctacatacctacattt

SEQ ID NO: 831 - DNA, 45 bp, Homo sapiens
  source 1..45: mol_type = genomic DNA
tgtgtggtttcccttgcagcaggaggaagctacatacctacattt

SEQ ID NO: 832 - DNA, 45 bp, Homo sapiens
  source 1..45: mol_type = genomic DNA
tgtgtggtgatgaccgcatcaggaggaagctacatacctacattt

SEQ ID NO: 833 - DNA, 45 bp, Homo sapiens
  source 1..45: mol_type = genomic DNA
tgtgtggtgacgcatgcatcaggaggaagctacatacctacattt

SEQ ID NO: 834 - DNA, 45 bp, Homo sapiens
  source 1..45: mol_type = genomic DNA
tgtgtggtgagctttgcatcaggaggaagctacatacctacattt

SEQ ID NO: 835 - DNA, 45 bp, Homo sapiens
  source 1..45: mol_type = genomic DNA
tgtgtggtgaaaagggcatcaggaggaagctacatacctacattt

SEQ ID NO: 836 - DNA, 45 bp, Homo sapiens
  source 1..45: mol_type = genomic DNA
tgtgtggtgaaacgggcatcaggaggaagctacatacctacattt

SEQ ID NO: 837 - DNA, 45 bp, Homo sapiens
  source 1..45: mol_type = genomic DNA
tgtgtggtgaccggtgcatcaggaggaagctacatacctacattt

SEQ ID NO: 838 - DNA, 45 bp, Homo sapiens
  source 1..45: mol_type = genomic DNA
tgtgtggtgaaaggagcatcaggaggaagctacatacctacattt

SEQ ID NO: 839 - DNA, 45 bp, Homo sapiens
  source 1..45: mol_type = genomic DNA
tgtgtggtgatgctggcatcaggaggaagctacatacctacattt

SEQ ID NO: 840 - DNA, 45 bp, Homo sapiens
  source 1..45: mol_type = genomic DNA
tgtgtggtgaacgaggcaacaggaggaagctacatacctacattt

SEQ ID NO: 841 - DNA, 45 bp, Homo sapiens
  source 1..45: mol_type = genomic DNA
tgtgtggtgtcccgcgcctcaggaggaagctacatacctacattt

SEQ ID NO: 842 - DNA, 45 bp, Homo sapiens
  source 1..45: mol_type = genomic DNA
tgtgtggtgacgctggcatcaggaggaagctacatacctacattt

SEQ ID NO: 843 - DNA, 45 bp, Homo sapiens
  source 1..45: mol_type = genomic DNA
tgtgtggtgaacatggccccaggaggaagctacatacctacattt

SEQ ID NO: 844 - DNA, 45 bp, Homo sapiens
  source 1..45: mol_type = genomic DNA
tgtgtggtgttacccgcatcaggaggaagctacatacctacattt

SEQ ID NO: 845 - DNA, 45 bp, Homo sapiens
  source 1..45: mol_type = genomic DNA
tgtgtggtgaataccgcatcaggaggaagctacatacctacattt

SEQ ID NO: 846 - DNA, 45 bp, Homo sapiens
  source 1..45: mol_type = genomic DNA
tgtgtggtgaactatgcatcaggaggaagctacatacctacattt

SEQ ID NO: 847 - DNA, 45 bp, Homo sapiens
  source 1..45: mol_type = genomic DNA
tgtgtggttccctttgcatcaggaggaagctacatacctacattt

SEQ ID NO: 848 - DNA, 45 bp, Homo sapiens
  source 1..45: mol_type = genomic DNA
tgtgtggttttcggtgcatcaggaggaagctacatacctacattt

SEQ ID NO: 849 - DNA, 45 bp, Homo sapiens
  source 1..45: mol_type = genomic DNA
tgtgtggtgagacaggcatcaggaggaagctacatacctacattt

SEQ ID NO: 850 - DNA, 45 bp, Homo sapiens
  source 1..45: mol_type = genomic DNA
tgtgtggttcagggggcatcaggaggaagctacatacctacattt

SEQ ID NO: 851 - DNA, 45 bp, Homo sapiens
  source 1..45: mol_type = genomic DNA
tgtgtggtgaggcctgcatcaggaggaagctacatacctacattt

SEQ ID NO: 852 - DNA, 45 bp, Homo sapiens
  source 1..45: mol_type = genomic DNA
tgtgtggtgaatggggcctcaggaggaagctacatacctacattt

SEQ ID NO: 853 - DNA, 45 bp, Homo sapiens
  source 1..45: mol_type = genomic DNA
tgtgtggtgaactccgcctcaggaggaagctacatacctacattt

SEQ ID NO: 854 - DNA, 45 bp, Homo sapiens
  source 1..45: mol_type = genomic DNA
tgtgtggtccgaatggcatcaggaggaagctacatacctacattt

SEQ ID NO: 855 - DNA, 45 bp, Homo sapiens
  source 1..45: mol_type = genomic DNA
tgtgtggtgaggtctgcatcaggaggaagctacatacctacattt

SEQ ID NO: 856 - DNA, 45 bp, Homo sapiens
  source 1..45: mol_type = genomic DNA
tgtgtggtgagggcggcatcaggaggaagctacatacctacattt

SEQ ID NO: 857 - DNA, 45 bp, Homo sapiens
  source 1..45: mol_type = genomic DNA
tgtgtggtgaacatggcccccggaggaagctacatacctacattt

SEQ ID NO: 858 - DNA, 45 bp, Homo sapiens
  source 1..45: mol_type = genomic DNA
tgtgtggtgatctcggcatcaggaggaagctacatacctacattt

SEQ ID NO: 859 - DNA, 45 bp, Homo sapiens
  source 1..45: mol_type = genomic DNA
tgtgtggtgattgttgcatcaggaggaagctacatacctacattt

SEQ ID NO: 860 - DNA, 45 bp, Homo sapiens
  source 1..45: mol_type = genomic DNA
tgtgtggtgacgtccgcatcaggaggaagctacatacctacattt

SEQ ID NO: 861 - DNA, 45 bp, Homo sapiens
  source 1..45: mol_type = genomic DNA
tgtgtggtgaactgggcgggaggaggaagctacatacctacattt

SEQ ID NO: 862 - DNA, 45 bp, Homo sapiens
  source 1..45: mol_type = genomic DNA
tgtgtggtgacctccgcatcaggaggaagctacatacctacattt

SEQ ID NO: 863 - DNA, 45 bp, Homo sapiens
  source 1..45: mol_type = genomic DNA
tgtgtggttacatatgcatcaggaggaagctacatacctacattt

SEQ ID NO: 864 - DNA, 45 bp, Homo sapiens
  source 1..45: mol_type = genomic DNA
tgtgtggtctacggagcatcaggaggaagctacatacctacattt

SEQ ID NO: 865 - DNA, 45 bp, Homo sapiens
  source 1..45: mol_type = genomic DNA
tgtgtggtgaccttggcatcaggaggaagctacatacctacattt

SEQ ID NO: 866 - DNA, 45 bp, Homo sapiens
  source 1..45: mol_type = genomic DNA
tgtgtggtgagtcccgcatcaggaggaagctacatacctacattt

SEQ ID NO: 867 - DNA, 45 bp, Homo sapiens
  source 1..45: mol_type = genomic DNA
tgtgtggtcggcgatgcatcaggaggaagctacatacctacattt

SEQ ID NO: 868 - DNA, 45 bp, Homo sapiens
  source 1..45: mol_type = genomic DNA
tgtgtggtgaacacagctcgaggaggaagctacatacctacattt

SEQ ID NO: 869 - DNA, 45 bp, Homo sapiens
  source 1..45: mol_type = genomic DNA
tgtgtggtctacttggcatcaggaggaagctacatacctacattt

SEQ ID NO: 870 - DNA, 45 bp, Homo sapiens
  source 1..45: mol_type = genomic DNA
tgtgtggtggttcatgcatcaggaggaagctacatacctacattt

SEQ ID NO: 871 - DNA, 45 bp, Homo sapiens
  source 1..45: mol_type = genomic DNA
tgtgtggtgagtacagcatcaggaggaagctacatacctacattt

SEQ ID NO: 872 - DNA, 45 bp, Homo sapiens
  source 1..45: mol_type = genomic DNA
tgtgtggtgaaccaggccacaggaggaagctacatacctacattt

SEQ ID NO: 873 - DNA, 45 bp, Homo sapiens
  source 1..45: mol_type = genomic DNA
tgtgtggtgatcaccgcatcaggaggaagctacatacctacattt

SEQ ID NO: 874 - DNA, 45 bp, Homo sapiens
  source 1..45: mol_type = genomic DNA
tgtgtggtgaacagagcgtcaggaggaagctacatacctacattt

SEQ ID NO: 875 - DNA, 45 bp, Homo sapiens
  source 1..45: mol_type = genomic DNA
tgtgtggtgcgtcaagcatcaggaggaagctacatacctacattt

SEQ ID NO: 876 - DNA, 45 bp, Homo sapiens
  source 1..45: mol_type = genomic DNA
tgtgtggtgcgcggagcatcaggaggaagctacatacctacattt

SEQ ID NO: 877 - DNA, 45 bp, Homo sapiens
  source 1..45: mol_type = genomic DNA
tgtgtggtggcctgggcatcaggaggaagctacatacctacattt

SEQ ID NO: 878 - DNA, 45 bp, Homo sapiens
  source 1..45: mol_type = genomic DNA
tgtgtggtgaggggtgcatcaggaggaagctacatacctacattt

SEQ ID NO: 879 - DNA, 45 bp, Homo sapiens
  source 1..45: mol_type = genomic DNA
tgtgtggtgaacggggcaccaggaggaagctacatacctacattt

SEQ ID NO: 880 - DNA, 45 bp, Homo sapiens
  source 1..45: mol_type = genomic DNA
tgtgtggtgaatttagcatcaggaggaagctacatacctacattt

SEQ ID NO: 881 - DNA, 45 bp, Homo sapiens
  source 1..45: mol_type = genomic DNA
tgtgtggtgtggaccgcatcaggaggaagctacatacctacattt

SEQ ID NO: 882 - DNA, 45 bp, Homo sapiens
  source 1..45: mol_type = genomic DNA
tgtgtggtgctacatgcatcaggaggaagctacatacctacattt

SEQ ID NO: 883 - DNA, 45 bp, Homo sapiens
  source 1..45: mol_type = genomic DNA
tgtgtggtgagactcgcatcaggaggaagctacatacctacattt

SEQ ID NO: 884 - DNA, 45 bp, Homo sapiens
  source 1..45: mol_type = genomic DNA
tgtgtggtgattgctgcatcaggaggaagctacatacctacattt

SEQ ID NO: 885 - DNA, 45 bp, Homo sapiens
  source 1..45: mol_type = genomic DNA
tgtgtggtgatctacgcatcaggaggaagctacatacctacattt

SEQ ID NO: 886 - DNA, 45 bp, Homo sapiens
  source 1..45: mol_type = genomic DNA
tgtgtggtgactatcgcatcaggaggaagctacatacctacattt

SEQ ID NO: 887 - DNA, 45 bp, Homo sapiens
  source 1..45: mol_type = genomic DNA
tgtgtggttcctcccgcatcaggaggaagctacatacctacattt

SEQ ID NO: 888 - DNA, 45 bp, Homo sapiens
  source 1..45: mol_type = genomic DNA
tgtgtggttagactcgcatcaggaggaagctacatacctacattt

SEQ ID NO: 889 - DNA, 45 bp, Homo sapiens
  source 1..45: mol_type = genomic DNA
tgtgtggtggaccatgcatcaggaggaagctacatacctacattt

SEQ ID NO: 890 - DNA, 45 bp, Homo sapiens
  source 1..45: mol_type = genomic DNA
tgtgtggtgattcacgcatcaggaggaagctacatacctacattt

SEQ ID NO: 891 - DNA, 45 bp, Homo sapiens
  source 1..45: mol_type = genomic DNA
tgtgtggtggacgacgcatcaggaggaagctacatacctacattt

SEQ ID NO: 892 - DNA, 45 bp, Homo sapiens
  source 1..45: mol_type = genomic DNA
tgtgtggtgaagggggccccaggaggaagctacatacctacattt

SEQ ID NO: 893 - DNA, 45 bp, Homo sapiens
  source 1..45: mol_type = genomic DNA
tgtgtggtctaccccgcatcaggaggaagctacatacctacattt

SEQ ID NO: 894 - DNA, 45 bp, Homo sapiens
  source 1..45: mol_type = genomic DNA
tgtgtggtgccgccagctccaggaggaagctacatacctacattt

SEQ ID NO: 895 - DNA, 45 bp, Homo sapiens
  source 1..45: mol_type = genomic DNA
tgtgtggtgtcccaggcatcaggaggaagctacatacctacattt

SEQ ID NO: 896 - DNA, 45 bp, Homo sapiens
  source 1..45: mol_type = genomic DNA
tgtgtggtgattttcgcatcaggaggaagctacatacctacattt

SEQ ID NO: 897 - DNA, 45 bp, Homo sapiens
  source 1..45: mol_type = genomic DNA
tgtgtggtgatcctggcatcaggaggaagctacatacctacattt

SEQ ID NO: 898 - DNA, 45 bp, Homo sapiens
  source 1..45: mol_type = genomic DNA
tgtgtggtgatcccggcgtcaggaggaagctacatacctacattt

SEQ ID NO: 899 - DNA, 45 bp, Homo sapiens
  source 1..45: mol_type = genomic DNA
tgtgtggtgactcccgcatcaggaggaagctacatacctacattt

SEQ ID NO: 900 - DNA, 45 bp, Homo sapiens
  source 1..45: mol_type = genomic DNA
tgtgtggtgaaccgggccccaggaggaagctacatacctacattt

SEQ ID NO: 901 - DNA, 45 bp, Homo sapiens
  source 1..45: mol_type = genomic DNA
tgtgtggtgaactcggccgaagggggaagctacatacctacattt

SEQ ID NO: 902 - DNA, 45 bp, Homo sapiens
  source 1..45: mol_type = genomic DNA
tgtgtggtcctccatgcatcaggaggaagctacatacctacattt

SEQ ID NO: 903 - DNA, 45 bp, Homo sapiens
  source 1..45: mol_type = genomic DNA
tgtgtggtgttaggggcatcaggaggaagctacatacctacattt

SEQ ID NO: 904 - DNA, 45 bp, Homo sapiens
  source 1..45: mol_type = genomic DNA
tgtgtggtgtactttgcatcaggaggaagctacatacctacattt

SEQ ID NO: 905 - DNA, 45 bp, Homo sapiens
  source 1..45: mol_type = genomic DNA
tgtgtggtgaacgaggcgtcaggaggaagctacatacctacattt

SEQ ID NO: 906 - DNA, 45 bp, Homo sapiens
  source 1..45: mol_type = genomic DNA
tgtgtggtgaacaacgcatcaggaggaagctacatacctacattt

SEQ ID NO: 907 - DNA, 45 bp, Homo sapiens
  source 1..45: mol_type = genomic DNA
tgtgtggtgagtttcgcatcaggaggaagctacatacctacattt

SEQ ID NO: 908 - DNA, 45 bp, Homo sapiens
  source 1..45: mol_type = genomic DNA
tgtgtggtgaaccgagcggcaggaggaagctacatacctacattt

SEQ ID NO: 909 - DNA, 45 bp, Homo sapiens
  source 1..45: mol_type = genomic DNA
tgtgtggtgaacggcgctccaggaggaagctacatacctacattt

SEQ ID NO: 910 - DNA, 45 bp, Homo sapiens
  source 1..45: mol_type = genomic DNA
tgtgtggtgaacatggcgccaggaggaagctacatacctacattt

SEQ ID NO: 911 - DNA, 45 bp, Homo sapiens
  source 1..45: mol_type = genomic DNA
tgtgtggtccgatccgcatcaggaggaagctacatacctacattt

SEQ ID NO: 912 - DNA, 45 bp, Homo sapiens
  source 1..45: mol_type = genomic DNA
tgtgtggtgattttggcatcaggaggaagctacatacctacattt

SEQ ID NO: 913 - DNA, 45 bp, Homo sapiens
  source 1..45: mol_type = genomic DNA
tgtgtggtgagcgcagcatcaggaggaagctacatacctacattt

SEQ ID NO: 914 - DNA, 45 bp, Homo sapiens
  source 1..45: mol_type = genomic DNA
tgtgtggtgaactcagcaaaaggaggaagctacatacctacattt

SEQ ID NO: 915 - DNA, 45 bp, Homo sapiens
  source 1..45: mol_type = genomic DNA
tgtgtggttctccttgcatcaggaggaagctacatacctacattt

SEQ ID NO: 916 - DNA, 45 bp, Homo sapiens
  source 1..45: mol_type = genomic DNA
tgtgtggtgcgaatggcatcaggaggaagctacatacctacattt

SEQ ID NO: 917 - DNA, 45 bp, Homo sapiens
  source 1..45: mol_type = genomic DNA
tgtgtggtgagatatgcatcaggaggaagctacatacctacattt

SEQ ID NO: 918 - DNA, 45 bp, Homo sapiens
  source 1..45: mol_type = genomic DNA
tgtgtggttatcccggcatcaggaggaagctacatacctacattt

SEQ ID NO: 919 - DNA, 45 bp, Homo sapiens
  source 1..45: mol_type = genomic DNA
tgtgtggtgaggcgtgcggcaggaggaagctacatacctacattt

SEQ ID NO: 920 - DNA, 45 bp, Homo sapiens
  source 1..45: mol_type = genomic DNA
tgtgtggtgcctgaagcatcaggaggaagctacatacctacattt

SEQ ID NO: 921 - DNA, 45 bp, Homo sapiens
  source 1..45: mol_type = genomic DNA
tgtgtggtgggccgggcatcaggaggaagctacatacctacattt

SEQ ID NO: 922 - DNA, 45 bp, Homo sapiens
  source 1..45: mol_type = genomic DNA
tgtgtggtgatgcgagcctcaggaggaagctacatacctacattt

SEQ ID NO: 923 - DNA, 45 bp, Homo sapiens
  source 1..45: mol_type = genomic DNA
tgtgtggtgaacggggcctcaggaggaagctacatacctacattt

SEQ ID NO: 924 - DNA, 45 bp, Homo sapiens
  source 1..45: mol_type = genomic DNA
tgtgtggtgaaggaggcatcaggaggaagctacatacctacattt

SEQ ID NO: 925 - DNA, 45 bp, Homo sapiens
  source 1..45: mol_type = genomic DNA
tgtgtggtaccccatgcatcaggaggaagctacatacctacattt

SEQ ID NO: 926 - DNA, 45 bp, Homo sapiens
  source 1..45: mol_type = genomic DNA
tgtgtggtgaacagcgcgggaggaggaagctacatacctacattt

SEQ ID NO: 927 - DNA, 45 bp, Homo sapiens
  source 1..45: mol_type = genomic DNA
tgtgtggtgatccgagcatcaggaggaagctacatacctacattt

SEQ ID NO: 928 - DNA, 45 bp, Homo sapiens
  source 1..45: mol_type = genomic DNA
tgtgtggtgaccgaggcgtcaggaggaagctacatacctacattt

SEQ ID NO: 929 - DNA, 45 bp, Homo sapiens
  source 1..45: mol_type = genomic DNA
tgtgtggtgaaatctgcatcaggaggaagctacatacctacattt

SEQ ID NO: 930 - DNA, 45 bp, Homo sapiens
  source 1..45: mol_type = genomic DNA
tgtgtggtgaacggggcggcaggaggaagctacatacctacattt

SEQ ID NO: 931 - DNA, 45 bp, Homo sapiens
  source 1..45: mol_type = genomic DNA
tgtgtggtgagcagggcatcaggaggaagctacatacctacattt

SEQ ID NO: 932 - DNA, 45 bp, Homo sapiens
  source 1..45: mol_type = genomic DNA
tgtgtggtgaaccccgcctcaggaggaagctacatacctacattt

SEQ ID NO: 933 - DNA, 45 bp, Homo sapiens
  source 1..45: mol_type = genomic DNA
tgtgtggtgaacagtgcgtcaggaggaagctacatacctacattt

SEQ ID NO: 934 - DNA, 45 bp, Homo sapiens
  source 1..45: mol_type = genomic DNA
tgtgtggtgaaggcggcatcaggaggaagctacatacctacattt

SEQ ID NO: 935 - DNA, 45 bp, Homo sapiens
  source 1..45: mol_type = genomic DNA
tgtgtggtgcccaccgcatcaggaggaagctacatacctacattt

SEQ ID NO: 936 - DNA, 45 bp, Homo sapiens
  source 1..45: mol_type = genomic DNA
tgtgtggtccgcgaggcttcaggaggaagctacatacctacattt

SEQ ID NO: 937 - DNA, 45 bp, Homo sapiens
  source 1..45: mol_type = genomic DNA
tgtgtggtggcccatgcatcaggaggaagctacatacctacattt

SEQ ID NO: 938 - DNA, 45 bp, Homo sapiens
  source 1..45: mol_type = genomic DNA
tgtgtggtgattggagcatcaggaggaagctacatacctacattt

SEQ ID NO: 939 - DNA, 45 bp, Homo sapiens
  source 1..45: mol_type = genomic DNA
tgtgtggtgaccggggcggcaggaggaagctacatacctacattt

SEQ ID NO: 940 - DNA, 45 bp, Homo sapiens
  source 1..45: mol_type = genomic DNA
tgtgtggtgagcgatgcatcaggaggaagctacatacctacattt

SEQ ID NO: 941 - DNA, 45 bp, Homo sapiens
  source 1..45: mol_type = genomic DNA
tgtgtggtgacgttggcgggaggaggaagctacatacctacattt

SEQ ID NO: 942 - DNA, 45 bp, Homo sapiens
  source 1..45: mol_type = genomic DNA
tgtgtggtgaacatggcggaaggaggaagctacatacctacattt

SEQ ID NO: 943 - DNA, 45 bp, Homo sapiens
  source 1..45: mol_type = genomic DNA
tgtgtggtgagtttagcatcaggaggaagctacatacctacattt

SEQ ID NO: 944 - DNA, 45 bp, Homo sapiens
  source 1..45: mol_type = genomic DNA
tgtgtggtgaggaccgccaggggaggaagctacatacctacattt

SEQ ID NO: 945 - DNA, 45 bp, Homo sapiens
  source 1..45: mol_type = genomic DNA
tgtgtggtgtctaaagcatcaggaggaagctacatacctacattt

SEQ ID NO: 946 - DNA, 45 bp, Homo sapiens
  source 1..45: mol_type = genomic DNA
tgtgtggtgagtgtcgcatcaggaggaagctacatacctacattt

SEQ ID NO: 947 - DNA, 45 bp, Homo sapiens
  source 1..45: mol_type = genomic DNA
tgtgtggtcccctccgcatcaggaggaagctacatacctacattt

SEQ ID NO: 948 - DNA, 45 bp, Homo sapiens
  source 1..45: mol_type = genomic DNA
tgtgtggtccagctcgcatcaggaggaagctacatacctacattt

SEQ ID NO: 949 - DNA, 45 bp, Homo sapiens
  source 1..45: mol_type = genomic DNA
tgtgtggtgagtacggcatcaggaggaagctacatacctacattt

SEQ ID NO: 950 - DNA, 45 bp, Homo sapiens
  source 1..45: mol_type = genomic DNA
tgtgtggtgaggatggcatcaggaggaagctacatacctacattt

SEQ ID NO: 951 - DNA, 45 bp, Homo sapiens
  source 1..45: mol_type = genomic DNA
tgtgtggtgagaatggcatcaggaggaagctacatacctacattt

SEQ ID NO: 952 - DNA, 45 bp, Homo sapiens
  source 1..45: mol_type = genomic DNA
tgtgtggtgaactcagccgcaggaggaagctacatacctacattt

SEQ ID NO: 953 - DNA, 45 bp, Homo sapiens
  source 1..45: mol_type = genomic DNA
tgtgtggtgaacatcgcagcaggaggaagctacatacctacattt

SEQ ID NO: 954 - DNA, 45 bp, Homo sapiens
  source 1..45: mol_type = genomic DNA
tgtgtggtgcggaccgcttcaggaggaagctacatacctacattt

SEQ ID NO: 955 - DNA, 45 bp, Homo sapiens
  source 1..45: mol_type = genomic DNA
tgtgtggtgcgacgagcatcaggaggaagctacatacctacattt

SEQ ID NO: 956 - DNA, 45 bp, Homo sapiens
  source 1..45: mol_type = genomic DNA
tgtgtggtgagcatcgcatcaggaggaagctacatacctacattt

SEQ ID NO: 957 - DNA, 45 bp, Homo sapiens
  source 1..45: mol_type = genomic DNA
tgtgtggtgtcccaggcggcaggaggaagctacatacctacattt

SEQ ID NO: 958 - DNA, 45 bp, Homo sapiens
  source 1..45: mol_type = genomic DNA
tgtgtggtgtccctagcatcaggaggaagctacatacctacattt

SEQ ID NO: 959 - DNA, 45 bp, Homo sapiens
  source 1..45: mol_type = genomic DNA
tgtgtggtgcacgtcgcatcaggaggaagctacatacctacattt

SEQ ID NO: 960 - DNA, 45 bp, Homo sapiens
  source 1..45: mol_type = genomic DNA
tgtgtggtgatcgtcgcatcaggaggaagctacatacctacattt

SEQ ID NO: 961 - DNA, 45 bp, Homo sapiens
  source 1..45: mol_type = genomic DNA
tgtgtggtgaccctcgccgggggaggaagctacatacctacattt

SEQ ID NO: 962 - DNA, 45 bp, Homo sapiens
  source 1..45: mol_type = genomic DNA
tgtgtggtgaggcttgccgggggaggaagctacatacctacattt

SEQ ID NO: 963 - DNA, 45 bp, Homo sapiens
  source 1..45: mol_type = genomic DNA
tgtgtggtgagtactgcatcaggaggaagctacatacctacattt

SEQ ID NO: 964 - DNA, 45 bp, Homo sapiens
  source 1..45: mol_type = genomic DNA
tgtgtggtgaggtcggcctcaggaggaagctacatacctacattt

SEQ ID NO: 965 - DNA, 45 bp, Homo sapiens
  source 1..45: mol_type = genomic DNA
tgtgtggtgaacgtcgcaacaggaggaagctacatacctacattt

SEQ ID NO: 966 - DNA, 45 bp, Homo sapiens
  source 1..45: mol_type = genomic DNA
tgtgtggtgaacatggccgcaggaggaagctacatacctacattt

SEQ ID NO: 967 - DNA, 45 bp, Homo sapiens
  source 1..45: mol_type = genomic DNA
tgtgtggtgaactcagcggcgggaggaagctacatacctacattt

SEQ ID NO: 968 - DNA, 45 bp, Homo sapiens
  source 1..45: mol_type = genomic DNA
tgtgtggtgtttaccgcatcaggaggaagctacatacctacattt

SEQ ID NO: 969 - DNA, 45 bp, Homo sapiens
  source 1..45: mol_type = genomic DNA
tgtgtggtgagaagtgcatcaggaggaagctacatacctacattt

SEQ ID NO: 970 - DNA, 45 bp, Homo sapiens
  source 1..45: mol_type = genomic DNA
tgtgtggtgtccggggcatcaggaggaagctacatacctacattt

SEQ ID NO: 971 - DNA, 45 bp, Homo sapiens
  source 1..45: mol_type = genomic DNA
tgtgtggtgaacgtcgcgacaggaggaagctacatacctacattt

SEQ ID NO: 972 - DNA, 45 bp, Homo sapiens
  source 1..45: mol_type = genomic DNA
tgtgtggtttccctcgcatcaggaggaagctacatacctacattt

SEQ ID NO: 973 - DNA, 45 bp, Homo sapiens
  source 1..45: mol_type = genomic DNA
tgtgtggtgtttgtggcatcaggaggaagctacatacctacattt

SEQ ID NO: 974 - DNA, 45 bp, Homo sapiens
  source 1..45: mol_type = genomic DNA
tgtgtggtgacctatgcatcaggaggaagctacatacctacattt

SEQ ID NO: 975 - DNA, 45 bp, Homo sapiens
  source 1..45: mol_type = genomic DNA
tgtgtggtttaccttgcatcaggaggaagctacatacctacattt

SEQ ID NO: 976 - DNA, 45 bp, Homo sapiens
  source 1..45: mol_type = genomic DNA
tgtgtggtgaatgtcgcagggggaggaagctacatacctacattt

SEQ ID NO: 977 - DNA, 45 bp, Homo sapiens
  source 1..45: mol_type = genomic DNA
tgtgtggtgaacatagccggaggaggaagctacatacctacattt

SEQ ID NO: 978 - DNA, 45 bp, Homo sapiens
  source 1..45: mol_type = genomic DNA
tgtgtggtgaatgaagcatcaggaggaagctacatacctacattt

SEQ ID NO: 979 - DNA, 45 bp, Homo sapiens
  source 1..45: mol_type = genomic DNA
tgtgtggtgatcctagcatcaggaggaagctacatacctacattt

SEQ ID NO: 980 - DNA, 45 bp, Homo sapiens
  source 1..45: mol_type = genomic DNA
tgtgtggttgtctttgcatcaggaggaagctacatacctacattt

SEQ ID NO: 981 - DNA, 45 bp, Homo sapiens
  source 1..45: mol_type = genomic DNA
tgtgtggtgaataaagcatcaggaggaagctacatacctacattt

SEQ ID NO: 982 - DNA, 45 bp, Homo sapiens
  source 1..45: mol_type = genomic DNA
tgtgtggtgagacttgcatcaggaggaagctacatacctacattt

SEQ ID NO: 983 - DNA, 45 bp, Homo sapiens
  source 1..45: mol_type = genomic DNA
tgtgtggtcgcccatgcatcaggaggaagctacatacctacattt

SEQ ID NO: 984 - DNA, 45 bp, Homo sapiens
  source 1..45: mol_type = genomic DNA
tgtgtggtcaggtctgcatcaggaggaagctacatacctacattt

SEQ ID NO: 985 - DNA, 45 bp, Homo sapiens
  source 1..45: mol_type = genomic DNA
tgtgtggtgccgaccgcatcaggaggaagctacatacctacattt

SEQ ID NO: 986 - DNA, 45 bp, Homo sapiens
  source 1..45: mol_type = genomic DNA
tgtgtggtgcgtagtgcatcaggaggaagctacatacctacattt

SEQ ID NO: 987 - DNA, 45 bp, Homo sapiens
  source 1..45: mol_type = genomic DNA
tgtgtggtgatcctagcttcaggaggaagctacatacctacattt

SEQ ID NO: 988 - DNA, 45 bp, Homo sapiens
  source 1..45: mol_type = genomic DNA
tgtgtggtgcgacatgcatcaggaggaagctacatacctacattt

SEQ ID NO: 989 - DNA, 45 bp, Homo sapiens
  source 1..45: mol_type = genomic DNA
tgtgtggttgcccatgcatcaggaggaagctacatacctacattt

SEQ ID NO: 990 - DNA, 45 bp, Homo sapiens
  source 1..45: mol_type = genomic DNA
tgtgtggtgattcccgcctcaggaggaagctacatacctacattt

SEQ ID NO: 991 - DNA, 45 bp, Homo sapiens
  source 1..45: mol_type = genomic DNA
tgtgtggtgacctccgcccgaggaggaagctacatacctacattt

SEQ ID NO: 992 - DNA, 45 bp, Homo sapiens
  source 1..45: mol_type = genomic DNA
tgtgtggtgaacatcgcttcaggaggaagctacatacctacattt

SEQ ID NO: 993 - DNA, 45 bp, Homo sapiens
  source 1..45: mol_type = genomic DNA
tgtgtggtgaacatcgccccaggaggaagctacatacctacattt

SEQ ID NO: 994 - DNA, 45 bp, Homo sapiens
  source 1..45: mol_type = genomic DNA
tgtgtggtgaacagggccggaggaggaagctacatacctacattt

SEQ ID NO: 995 - DNA, 45 bp, Homo sapiens
  source 1..45: mol_type = genomic DNA
tgtgtggtgaacaaggcctcaggaggaagctacatacctacattt

SEQ ID NO: 996 - DNA, 45 bp, Homo sapiens
  source 1..45: mol_type = genomic DNA
tgtgtggtttttcatgcatcaggaggaagctacatacctacattt

SEQ ID NO: 997 - DNA, 45 bp, Homo sapiens
  source 1..45: mol_type = genomic DNA
tgtgtggtgataagggcatcaggaggaagctacatacctacattt

SEQ ID NO: 998 - DNA, 45 bp, Homo sapiens
  source 1..45: mol_type = genomic DNA
tgtgtggtgacccgtgcatcaggaggaagctacatacctacattt

SEQ ID NO: 999 - DNA, 45 bp, Homo sapiens
  source 1..45: mol_type = genomic DNA
tgtgtggtgaacaacgcaagaggaggaagctacatacctacattt

SEQ ID NO: 1000 - DNA, 45 bp, Homo sapiens
  source 1..45: mol_type = genomic DNA
tgtgtggtttcccgagcatcaggaggaagctacatacctacattt

SEQ ID NO: 1001 - DNA, 45 bp, Homo sapiens
  source 1..45: mol_type = genomic DNA
tgtgtggtgaccgcggcatcaggaggaagctacatacctacattt

SEQ ID NO: 1002 - DNA, 45 bp, Homo sapiens
  source 1..45: mol_type = genomic DNA
tgtgtggtgaacattgcggcaggaggaagctacatacctacattt

SEQ ID NO: 1003 - DNA, 45 bp, Homo sapiens
  source 1..45: mol_type = genomic DNA
tgtgtggtcccccatgcatcaggaggaagctacatacctacattt

SEQ ID NO: 1004 - DNA, 45 bp, Homo sapiens
  source 1..45: mol_type = genomic DNA
tgtgtggtgaacctcgcctcaggaggaagctacatacctacattt

SEQ ID NO: 1005 - DNA, 45 bp, Homo sapiens
  source 1..45: mol_type = genomic DNA
tgtgtggtgcggggagcatcaggaggaagctacatacctacattt

SEQ ID NO: 1006 - DNA, 45 bp, Homo sapiens
  source 1..45: mol_type = genomic DNA
tgtgtggtgccacatgcatcaggaggaagctacatacctacattt

SEQ ID NO: 1007 - DNA, 45 bp, Homo sapiens
  source 1..45: mol_type = genomic DNA
tgtgtggtgccctctgcatcaggaggaagctacatacctacattt

SEQ ID NO: 1008 - DNA, 45 bp, Homo sapiens
  source 1..45: mol_type = genomic DNA
tgtgtggtctccgttgcatcaggaggaagctacatacctacattt

SEQ ID NO: 1009 - DNA, 45 bp, Homo sapiens
  source 1..45: mol_type = genomic DNA
tgtgtggtgagtggggcgcagggaggaagctacatacctacattt

SEQ ID NO: 1010 - DNA, 45 bp, Homo sapiens
  source 1..45: mol_type = genomic DNA
tgtgtggttcgatccgcatcaggaggaagctacatacctacattt

SEQ ID NO: 1011 - DNA, 45 bp, Homo sapiens
  source 1..45: mol_type = genomic DNA
tgtgtggtgagaaaagcatcaggaggaagctacatacctacattt

SEQ ID NO: 1012 - DNA, 45 bp, Homo sapiens
  source 1..45: mol_type = genomic DNA
tgtgtggtgaacatcgcagaaggaggaagctacatacctacattt

SEQ ID NO: 1013 - DNA, 45 bp, Homo sapiens
  source 1..45: mol_type = genomic DNA
tgtgtggtgagggctgcatcaggaggaagctacatacctacattt

SEQ ID NO: 1014 - DNA, 45 bp, Homo sapiens
  source 1..45: mol_type = genomic DNA
tgtgtggtgaatatcgcatcaggaggaagctacatacctacattt

SEQ ID NO: 1015 - DNA, 45 bp, Homo sapiens
  source 1..45: mol_type = genomic DNA
tgtgtggtgaagaccgcatcaggaggaagctacatacctacattt

SEQ ID NO: 1016 - DNA, 45 bp, Homo sapiens
  source 1..45: mol_type = genomic DNA
tgtgtggtgaatagtgccgtaggaggaagctacatacctacattt

SEQ ID NO: 1017 - DNA, 45 bp, Homo sapiens
  source 1..45: mol_type = genomic DNA
tgtgtggtgaggagggcatcaggaggaagctacatacctacattt

SEQ ID NO: 1018 - DNA, 45 bp, Homo sapiens
  source 1..45: mol_type = genomic DNA
tgtgtggtgaacatggccggtggaggaagctacatacctacattt

SEQ ID NO: 1019 - DNA, 45 bp, Homo sapiens
  source 1..45: mol_type = genomic DNA
tgtgtggtgggcacggcatcaggaggaagctacatacctacattt

SEQ ID NO: 1020 - DNA, 45 bp, Homo sapiens
  source 1..45: mol_type = genomic DNA
tgtgtggtgagcagtgcatcaggaggaagctacatacctacattt

SEQ ID NO: 1021 - DNA, 45 bp, Homo sapiens
  source 1..45: mol_type = genomic DNA
tgtgtggtgaacatggcgaggggaggaagctacatacctacattt

SEQ ID NO: 1022 - DNA, 45 bp, Homo sapiens
  source 1..45: mol_type = genomic DNA
tgtgtggtgaaggttgcatcaggaggaagctacatacctacattt

SEQ ID NO: 1023 - DNA, 45 bp, Homo sapiens
  source 1..45: mol_type = genomic DNA
tgtgtggtaaaacatgcatcaggaggaagctacatacctacattt

SEQ ID NO: 1024 - DNA, 45 bp, Homo sapiens
  source 1..45: mol_type = genomic DNA
tgtgtggtcagagcggcatcaggaggaagctacatacctacattt

SEQ ID NO: 1025 - DNA, 45 bp, Homo sapiens
  source 1..45: mol_type = genomic DNA
tgtgtggtgaacatggcagccggaggaagctacatacctacattt

SEQ ID NO: 1026 - DNA, 45 bp, Homo sapiens
  source 1..45: mol_type = genomic DNA
tgtgtggtgagatccgcatcaggaggaagctacatacctacattt

SEQ ID NO: 1027 - DNA, 45 bp, Homo sapiens
  source 1..45: mol_type = genomic DNA
tgtgtggtgaacatagcactcggaggaagctacatacctacattt

SEQ ID NO: 1028 - DNA, 45 bp, Homo sapiens
  source 1..45: mol_type = genomic DNA
tgtgtggtgtcccaggcggggggaggaagctacatacctacattt

SEQ ID NO: 1029 - DNA, 45 bp, Homo sapiens
  source 1..45: mol_type = genomic DNA
tgtgtggtgaccttggcggggggaggaagctacatacctacattt

SEQ ID NO: 1030 - DNA, 45 bp, Homo sapiens
  source 1..45: mol_type = genomic DNA
tgtgtggtgagagtcgcatcaggaggaagctacatacctacattt

SEQ ID NO: 1031 - DNA, 45 bp, Homo sapiens
  source 1..45: mol_type = genomic DNA
tgtgtggtgtctactgcatcaggaggaagctacatacctacattt

SEQ ID NO: 1032 - DNA, 45 bp, Homo sapiens
  source 1..45: mol_type = genomic DNA
tgtgtggtgcgtttggccggggggggaagctacatacctacattt

SEQ ID NO: 1033 - DNA, 45 bp, Homo sapiens
  source 1..45: mol_type = genomic DNA
tgtgtggtgcggactgcatcaggaggaagctacatacctacattt

SEQ ID NO: 1034 - DNA, 45 bp, Homo sapiens
  source 1..45: mol_type = genomic DNA
tgtgtggtgaacgtcgcggccggaggaagctacatacctacattt

SEQ ID NO: 1035 - DNA, 45 bp, Homo sapiens
  source 1..45: mol_type = genomic DNA
tgtgtggtggccgacgcatcaggaggaagctacatacctacattt

SEQ ID NO: 1036 - DNA, 45 bp, Homo sapiens
  source 1..45: mol_type = genomic DNA
tgtgtggtgaacagggcgccaggaggaagctacatacctacattt

SEQ ID NO: 1037 - DNA, 45 bp, Homo sapiens
  source 1..45: mol_type = genomic DNA
tgtgtggtgactctggcatcaggaggaagctacatacctacattt

SEQ ID NO: 1038 - DNA, 45 bp, Homo sapiens
  source 1..45: mol_type = genomic DNA
tgtgtggtggttctcgcatcaggaggaagctacatacctacattt

SEQ ID NO: 1039 - DNA, 45 bp, Homo sapiens
  source 1..45: mol_type = genomic DNA
tgtgtggtgaggggggcggagggaggaagctacatacctacattt

SEQ ID NO: 1040 - DNA, 45 bp, Homo sapiens
  source 1..45: mol_type = genomic DNA
tgtgtggtgaacggggctacaggaggaagctacatacctacattt

SEQ ID NO: 1041 - DNA, 45 bp, Homo sapiens
  source 1..45: mol_type = genomic DNA
tgtgtggtgtctgtagcatcaggaggaagctacatacctacattt

SEQ ID NO: 1042 - DNA, 45 bp, Homo sapiens
  source 1..45: mol_type = genomic DNA
tgtgtggtgactccagcatcaggaggaagctacatacctacattt

SEQ ID NO: 1043 - DNA, 45 bp, Homo sapiens
  source 1..45: mol_type = genomic DNA
tgtgtggtgatgggcgcatcaggaggaagctacatacctacattt

SEQ ID NO: 1044 - DNA, 45 bp, Homo sapiens
  source 1..45: mol_type = genomic DNA
tgtgtggtgatccgcgcatcaggaggaagctacatacctacattt

SEQ ID NO: 1045 - DNA, 45 bp, Homo sapiens
  source 1..45: mol_type = genomic DNA
tgtgtggtgagtgatgcatcaggaggaagctacatacctacattt

SEQ ID NO: 1046 - DNA, 45 bp, Homo sapiens
  source 1..45: mol_type = genomic DNA
tgtgtggtccagcatgcatcaggaggaagctacatacctacattt

SEQ ID NO: 1047 - DNA, 45 bp, Homo sapiens
  source 1..45: mol_type = genomic DNA
tgtgtggtgaagagggcctcaggaggaagctacatacctacattt

SEQ ID NO: 1048 - DNA, 45 bp, Homo sapiens
  source 1..45: mol_type = genomic DNA
tgtgtggtcctcctcgcatcaggaggaagctacatacctacattt

SEQ ID NO: 1049 - DNA, 45 bp, Homo sapiens
  source 1..45: mol_type = genomic DNA
tgtgtggtgatgggggcatcaggaggaagctacatacctacattt

SEQ ID NO: 1050 - DNA, 45 bp, Homo sapiens
  source 1..45: mol_type = genomic DNA
tgtgtggtgaagagtgcatcaggaggaagctacatacctacattt

SEQ ID NO: 1051 - DNA, 45 bp, Homo sapiens
  source 1..45: mol_type = genomic DNA
tgtgtggtggaccaagcatcaggaggaagctacatacctacattt

SEQ ID NO: 1052 - DNA, 45 bp, Homo sapiens
  source 1..45: mol_type = genomic DNA
tgtgtggtgaactgggctgcaggaggaagctacatacctacattt

SEQ ID NO: 1053 - DNA, 45 bp, Homo sapiens
  source 1..45: mol_type = genomic DNA
tgtgtggtgaaagatgcatcaggaggaagctacatacctacattt

SEQ ID NO: 1054 - DNA, 45 bp, Homo sapiens
  source 1..45: mol_type = genomic DNA
tgtgtggtgtcccaagcatcaggaggaagctacatacctacattt

SEQ ID NO: 1055 - DNA, 45 bp, Homo sapiens
  source 1..45: mol_type = genomic DNA
tgtgtggtgaacctcgcagcaggaggaagctacatacctacattt

SEQ ID NO: 1056 - DNA, 45 bp, Homo sapiens
  source 1..45: mol_type = genomic DNA
tgtgtggtcacgggggcatcaggaggaagctacatacctacattt

SEQ ID NO: 1057 - DNA, 45 bp, Homo sapiens
  source 1..45: mol_type = genomic DNA
tgtgtggtgaacgtcgcaccaggaggaagctacatacctacattt

SEQ ID NO: 1058 - DNA, 45 bp, Homo sapiens
  source 1..45: mol_type = genomic DNA
tgtgtggtgattgcagcatcaggaggaagctacatacctacattt

SEQ ID NO: 1059 - DNA, 45 bp, Homo sapiens
  source 1..45: mol_type = genomic DNA
tgtgtggtgatacatgcatcaggaggaagctacatacctacattt

SEQ ID NO: 1060 - DNA, 45 bp, Homo sapiens
  source 1..45: mol_type = genomic DNA
tgtgtggtgaatccagcatcaggaggaagctacatacctacattt

SEQ ID NO: 1061 - DNA, 45 bp, Homo sapiens
  source 1..45: mol_type = genomic DNA
tgtgtggtgaagcccgcatcaggaggaagctacatacctacattt

SEQ ID NO: 1062 - DNA, 45 bp, Homo sapiens
  source 1..45: mol_type = genomic DNA
tgtgtggtgaagggggcaggaggaggaagctacatacctacattt

SEQ ID NO: 1063 - DNA, 45 bp, Homo sapiens
  source 1..45: mol_type = genomic DNA
tgtgtggtgccgggggcatcaggaggaagctacatacctacattt

SEQ ID NO: 1064 - DNA, 45 bp, Homo sapiens
  source 1..45: mol_type = genomic DNA
tgtgtggttttcggggcatcaggaggaagctacatacctacattt

SEQ ID NO: 1065 - DNA, 45 bp, Homo sapiens
  source 1..45: mol_type = genomic DNA
tgtgtggtgtcccatgcctcaggaggaagctacatacctacattt

SEQ ID NO: 1066 - DNA, 45 bp, Homo sapiens
  source 1..45: mol_type = genomic DNA
tgtgtggtggccgtagcatcaggaggaagctacatacctacattt

SEQ ID NO: 1067 - DNA, 45 bp, Homo sapiens
  source 1..45: mol_type = genomic DNA
tgtgtggtgacccttgcagggggaggaagctacatacctacattt

SEQ ID NO: 1068 - DNA, 45 bp, Homo sapiens
  source 1..45: mol_type = genomic DNA
tgtgtggtgacaaacgcaccaggaggaagctacatacctacattt

SEQ ID NO: 1069 - DNA, 45 bp, Homo sapiens
  source 1..45: mol_type = genomic DNA
tgtgtggtgaacttggccgtgggaggaagctacatacctacattt

SEQ ID NO: 1070 - DNA, 45 bp, Homo sapiens
  source 1..45: mol_type = genomic DNA
tgtgtggtgaacgctgcagcaggaggaagctacatacctacattt

SEQ ID NO: 1071 - DNA, 45 bp, Homo sapiens
  source 1..45: mol_type = genomic DNA
tgtgtggtgaacacagcatcaggaggaagctacatacctacattt

SEQ ID NO: 1072 - DNA, 45 bp, Homo sapiens
  source 1..45: mol_type = genomic DNA
tgtgtggtgaatcccgcatcaggaggaagctacatacctacattt

SEQ ID NO: 1073 - DNA, 45 bp, Homo sapiens
  source 1..45: mol_type = genomic DNA
tgtgtggtcctccctgcatcaggaggaagctacatacctacattt

SEQ ID NO: 1074 - DNA, 45 bp, Homo sapiens
  source 1..45: mol_type = genomic DNA
tgtgtggtgagggacgcatcaggaggaagctacatacctacattt

SEQ ID NO: 1075 - DNA, 45 bp, Homo sapiens
  source 1..45: mol_type = genomic DNA
tgtgtggtgacggctgcatcaggaggaagctacatacctacattt

SEQ ID NO: 1076 - DNA, 45 bp, Homo sapiens
  source 1..45: mol_type = genomic DNA
tgtgtggtccacgatgcatcaggaggaagctacatacctacattt

SEQ ID NO: 1077 - DNA, 45 bp, Homo sapiens
  source 1..45: mol_type = genomic DNA
tgtgtggtgatcgttgcatcaggaggaagctacatacctacattt

SEQ ID NO: 1078 - DNA, 45 bp, Homo sapiens
  source 1..45: mol_type = genomic DNA
tgtgtggtgaaccttgccgcaggaggaagctacatacctacattt

SEQ ID NO: 1079 - DNA, 45 bp, Homo sapiens
  source 1..45: mol_type = genomic DNA
tgtgtggtgaacgaagcttcaggaggaagctacatacctacattt

SEQ ID NO: 1080 - DNA, 45 bp, Homo sapiens
  source 1..45: mol_type = genomic DNA
tgtgtggtgattttagcatcaggaggaagctacatacctacattt

SEQ ID NO: 1081 - DNA, 45 bp, Homo sapiens
  source 1..45: mol_type = genomic DNA
tgtgtggtgactgtcgcatcaggaggaagctacatacctacattt

SEQ ID NO: 1082 - DNA, 45 bp, Homo sapiens
  source 1..45: mol_type = genomic DNA
tgtgtggtgaacatcgcccgcggaggaagctacatacctacattt

SEQ ID NO: 1083 - DNA, 45 bp, Homo sapiens
  source 1..45: mol_type = genomic DNA
tgtgtggtgaacatagccccaggaggaagctacatacctacattt

SEQ ID NO: 1084 - DNA, 45 bp, Homo sapiens
  source 1..45: mol_type = genomic DNA
tgtgtggtgaacattgcagcaggaggaagctacatacctacattt

SEQ ID NO: 1085 - DNA, 45 bp, Homo sapiens
  source 1..45: mol_type = genomic DNA
tgtgtggtgaacccggcaacaggaggaagctacatacctacattt

SEQ ID NO: 1086 - DNA, 45 bp, Homo sapiens
  source 1..45: mol_type = genomic DNA
tgtgtggtgaacaaggcagcaggaggaagctacatacctacattt

SEQ ID NO: 1087 - DNA, 45 bp, Homo sapiens
  source 1..45: mol_type = genomic DNA
tgtgtggtgaaacctgcatcaggaggaagctacatacctacattt

SEQ ID NO: 1088 - DNA, 45 bp, Homo sapiens
  source 1..45: mol_type = genomic DNA
tgtgtggttggggtggcggcaggaggaagctacatacctacattt

SEQ ID NO: 1089 - DNA, 45 bp, Homo sapiens
  source 1..45: mol_type = genomic DNA
tgtgtggtctatctagcatcaggaggaagctacatacctacattt

SEQ ID NO: 1090 - DNA, 45 bp, Homo sapiens
  source 1..45: mol_type = genomic DNA
tgtgtggtgtctcatgcatcaggaggaagctacatacctacattt

SEQ ID NO: 1091 - DNA, 45 bp, Homo sapiens
  source 1..45: mol_type = genomic DNA
tgtgtggtggcacatgcatcaggaggaagctacatacctacattt

SEQ ID NO: 1092 - DNA, 45 bp, Homo sapiens
  source 1..45: mol_type = genomic DNA
tgtgtggttcttcatgcatcaggaggaagctacatacctacattt

SEQ ID NO: 1093 - DNA, 45 bp, Homo sapiens
  source 1..45: mol_type = genomic DNA
tgtgtggtgaccggagcatcaggaggaagctacatacctacattt

SEQ ID NO: 1094 - DNA, 45 bp, Homo sapiens
  source 1..45: mol_type = genomic DNA
tgtgtggtgaatgatgcatcaggaggaagctacatacctacattt

SEQ ID NO: 1095 - DNA, 45 bp, Homo sapiens
  source 1..45: mol_type = genomic DNA
tgtgtggtgaactccgccacaggaggaagctacatacctacattt

SEQ ID NO: 1096 - DNA, 45 bp, Homo sapiens
  source 1..45: mol_type = genomic DNA
tgtgtggtgaagcttgcatcaggaggaagctacatacctacattt

SEQ ID NO: 1097 - DNA, 45 bp, Homo sapiens
  source 1..45: mol_type = genomic DNA
tgtgtggtgatcatagcctcaggaggaagctacatacctacattt

SEQ ID NO: 1098 - DNA, 45 bp, Homo sapiens
  source 1..45: mol_type = genomic DNA
tgtgtggtgtcctccgcatcaggaggaagctacatacctacattt

SEQ ID NO: 1099 - DNA, 45 bp, Homo sapiens
  source 1..45: mol_type = genomic DNA
tgtgtggtgatttcagcatcaggaggaagctacatacctacattt

SEQ ID NO: 1100 - DNA, 45 bp, Homo sapiens
  source 1..45: mol_type = genomic DNA
tgtgtggtgagcgctgcatcaggaggaagctacatacctacattt

SEQ ID NO: 1101 - DNA, 45 bp, Homo sapiens
  source 1..45: mol_type = genomic DNA
tgtgtggtgaagggggcatcaggaggaagctacatacctacattt

SEQ ID NO: 1102 - DNA, 45 bp, Homo sapiens
  source 1..45: mol_type = genomic DNA
tgtgtggtgaaccgtgccccaggaggaagctacatacctacattt

SEQ ID NO: 1103 - DNA, 45 bp, Homo sapiens
  source 1..45: mol_type = genomic DNA
tgtgtggtgaagtacgcatcaggaggaagctacatacctacattt

SEQ ID NO: 1104 - DNA, 45 bp, Homo sapiens
  source 1..45: mol_type = genomic DNA
tgtgtggtggtacatgcatcaggaggaagctacatacctacattt

SEQ ID NO: 1105 - DNA, 45 bp, Homo sapiens
  source 1..45: mol_type = genomic DNA
tgtgtggtcaacggggcatcaggaggaagctacatacctacattt

SEQ ID NO: 1106 - DNA, 45 bp, Homo sapiens
  source 1..45: mol_type = genomic DNA
tgtgtggtgacacaggcatcaggaggaagctacatacctacattt

SEQ ID NO: 1107 - DNA, 45 bp, Homo sapiens
  source 1..45: mol_type = genomic DNA
tgtgtggtgcccctggcgacaggaggaagctacatacctacattt

SEQ ID NO: 1108 - DNA, 45 bp, Homo sapiens
  source 1..45: mol_type = genomic DNA
tgtgtggtgattcttgcatcaggaggaagctacatacctacattt

SEQ ID NO: 1109 - DNA, 45 bp, Homo sapiens
  source 1..45: mol_type = genomic DNA
tgtgtggtgaacgaggcgggaggaggaagctacatacctacattt

SEQ ID NO: 1110 - DNA, 45 bp, Homo sapiens
  source 1..45: mol_type = genomic DNA
tgtgtggtgattcccgcatcaggaggaagctacatacctacattt

SEQ ID NO: 1111 - DNA, 45 bp, Homo sapiens
  source 1..45: mol_type = genomic DNA
tgtgtggtgattgcggcatcaggaggaagctacatacctacattt

SEQ ID NO: 1112 - DNA, 45 bp, Homo sapiens
  source 1..45: mol_type = genomic DNA
tgtgtggtgcgggttgcatcaggaggaagctacatacctacattt

SEQ ID NO: 1113 - DNA, 45 bp, Homo sapiens
  source 1..45: mol_type = genomic DNA
tgtgtggtgaacagggccccaggaggaagctacatacctacattt

SEQ ID NO: 1114 - DNA, 45 bp, Homo sapiens
  source 1..45: mol_type = genomic DNA
tgtgtggtggagcaagcatcaggaggaagctacatacctacattt

SEQ ID NO: 1115 - DNA, 45 bp, Homo sapiens
  source 1..45: mol_type = genomic DNA
tgtgtggtccgggcggcatcaggaggaagctacatacctacattt

SEQ ID NO: 1116 - DNA, 45 bp, Homo sapiens
  source 1..45: mol_type = genomic DNA
tgtgtggtctacatggccccgggaggaagctacatacctacattt

SEQ ID NO: 1117 - DNA, 45 bp, Homo sapiens
  source 1..45: mol_type = genomic DNA
tgtgtggtccgcatagcatcaggaggaagctacatacctacattt

SEQ ID NO: 1118 - DNA, 45 bp, Homo sapiens
  source 1..45: mol_type = genomic DNA
tgtgtggtgatcttcgcatcaggaggaagctacatacctacattt

SEQ ID NO: 1119 - DNA, 45 bp, Homo sapiens
  source 1..45: mol_type = genomic DNA
tgtgtggtgtcccgtgcatcaggaggaagctacatacctacattt

SEQ ID NO: 1120 - DNA, 45 bp, Homo sapiens
  source 1..45: mol_type = genomic DNA
tgtgtggtgcgtggggcatcaggaggaagctacatacctacattt

SEQ ID NO: 1121 - DNA, 45 bp, Homo sapiens
  source 1..45: mol_type = genomic DNA
tgtgtggtgactgttgcatcaggaggaagctacatacctacattt

SEQ ID NO: 1122 - DNA, 45 bp, Homo sapiens
  source 1..45: mol_type = genomic DNA
tgtgtggtgtccgttgcatcaggaggaagctacatacctacattt

SEQ ID NO: 1123 - DNA, 45 bp, Homo sapiens
  source 1..45: mol_type = genomic DNA
tgtgtggtgaacatcgcaggaggaggaagctacatacctacattt

SEQ ID NO: 1124 - DNA, 45 bp, Homo sapiens
  source 1..45: mol_type = genomic DNA
tgtgtggtccggggggcatcaggaggaagctacatacctacattt

SEQ ID NO: 1125 - DNA, 45 bp, Homo sapiens
  source 1..45: mol_type = genomic DNA
tgtgtggtatttcatgcatcaggaggaagctacatacctacattt

SEQ ID NO: 1126 - DNA, 45 bp, Homo sapiens
  source 1..45: mol_type = genomic DNA
tgtgtggtgagccctgcatcaggaggaagctacatacctacattt

SEQ ID NO: 1127 - DNA, 45 bp, Homo sapiens
  source 1..45: mol_type = genomic DNA
tgtgtggtgtccaccgcatcaggaggaagctacatacctacattt

SEQ ID NO: 1128 - DNA, 45 bp, Homo sapiens
  source 1..45: mol_type = genomic DNA
tgtgtggtgaacttggcgtcaggaggaagctacatacctacattt

SEQ ID NO: 1129 - DNA, 45 bp, Homo sapiens
  source 1..45: mol_type = genomic DNA
tgtgtggtgaggagagcatcaggaggaagctacatacctacattt

SEQ ID NO: 1130 - DNA, 45 bp, Homo sapiens
  source 1..45: mol_type = genomic DNA
tgtgtggtgagttcagcatcaggaggaagctacatacctacattt

SEQ ID NO: 1131 - DNA, 45 bp, Homo sapiens
  source 1..45: mol_type = genomic DNA
tgtgtggttcgcctcgcatcaggaggaagctacatacctacattt

SEQ ID NO: 1132 - DNA, 45 bp, Homo sapiens
  source 1..45: mol_type = genomic DNA
tgtgtggtgaacagggcggcaggaggaagctacatacctacattt

SEQ ID NO: 1133 - DNA, 45 bp, Homo sapiens
  source 1..45: mol_type = genomic DNA
tgtgtggtgacgcctgcatcaggaggaagctacatacctacattt

SEQ ID NO: 1134 - DNA, 45 bp, Homo sapiens
  source 1..45: mol_type = genomic DNA
tgtgtggtgaaccgggccacaggaggaagctacatacctacattt

SEQ ID NO: 1135 - DNA, 45 bp, Homo sapiens
  source 1..45: mol_type = genomic DNA
tgtgtggtctccctagcgggaggaggaagctacatacctacattt

SEQ ID NO: 1136 - DNA, 45 bp, Homo sapiens
  source 1..45: mol_type = genomic DNA
tgtgtggtgagccctgcttcaggaggaagctacatacctacattt

SEQ ID NO: 1137 - DNA, 45 bp, Homo sapiens
  source 1..45: mol_type = genomic DNA
tgtgtggtgtctagtgcatcaggaggaagctacatacctacattt

SEQ ID NO: 1138 - DNA, 45 bp, Homo sapiens
  source 1..45: mol_type = genomic DNA
tgtgtggtggcccttgcatcaggaggaagctacatacctacattt

SEQ ID NO: 1139 - DNA, 45 bp, Homo sapiens
  source 1..45: mol_type = genomic DNA
tgtgtggtgatgcatgcatcaggaggaagctacatacctacattt

SEQ ID NO: 1140 - DNA, 45 bp, Homo sapiens
  source 1..45: mol_type = genomic DNA
tgtgtggtgaaccaggcctcaggaggaagctacatacctacattt

SEQ ID NO: 1141 - DNA, 45 bp, Homo sapiens
  source 1..45: mol_type = genomic DNA
tgtgtggttatgaaggcggaaggaggaagctacatacctacattt

SEQ ID NO: 1142 - DNA, 45 bp, Homo sapiens
  source 1..45: mol_type = genomic DNA
tgtgtggtgaaccccgcttcaggaggaagctacatacctacattt

SEQ ID NO: 1143 - DNA, 45 bp, Homo sapiens
  source 1..45: mol_type = genomic DNA
tgtgtggtgaacggagcagtaggaggaagctacatacctacattt

SEQ ID NO: 1144 - DNA, 45 bp, Homo sapiens
  source 1..45: mol_type = genomic DNA
tgtgtggtccgttctgcatcaggaggaagctacatacctacattt

SEQ ID NO: 1145 - DNA, 45 bp, Homo sapiens
  source 1..45: mol_type = genomic DNA
tgtgtggtgattaatgcatcaggaggaagctacatacctacattt

SEQ ID NO: 1146 - DNA, 45 bp, Homo sapiens
  source 1..45: mol_type = genomic DNA
tgtgtggtgaccagtgcatcaggaggaagctacatacctacattt

SEQ ID NO: 1147 - DNA, 45 bp, Homo sapiens
  source 1..45: mol_type = genomic DNA
tgtgtggtgagctcggcatcaggaggaagctacatacctacattt

SEQ ID NO: 1148 - DNA, 45 bp, Homo sapiens
  source 1..45: mol_type = genomic DNA
tgtgtggttcccccggcatcaggaggaagctacatacctacattt

SEQ ID NO: 1149 - DNA, 45 bp, Homo sapiens
  source 1..45: mol_type = genomic DNA
tgtgtggtccgtagggcatcaggaggaagctacatacctacattt

SEQ ID NO: 1150 - DNA, 45 bp, Homo sapiens
  source 1..45: mol_type = genomic DNA
tgtgtggtgtcctcagcatcaggaggaagctacatacctacattt

SEQ ID NO: 1151 - DNA, 45 bp, Homo sapiens
  source 1..45: mol_type = genomic DNA
tgtgtggtgagagtagcatcaggaggaagctacatacctacattt

SEQ ID NO: 1152 - DNA, 45 bp, Homo sapiens
  source 1..45: mol_type = genomic DNA
tgtgtggtggcccaggcatcaggaggaagctacatacctacattt

SEQ ID NO: 1153 - DNA, 45 bp, Homo sapiens
  source 1..45: mol_type = genomic DNA
tgtgtggtgtctctagcatcaggaggaagctacatacctacattt

SEQ ID NO: 1154 - DNA, 45 bp, Homo sapiens
  source 1..45: mol_type = genomic DNA
tgtgtggtgaactctgcgacaggaggaagctacatacctacattt

SEQ ID NO: 1155 - DNA, 45 bp, Homo sapiens
  source 1..45: mol_type = genomic DNA
tgtgtggtgagtgtggcatcaggaggaagctacatacctacattt

SEQ ID NO: 1156 - DNA, 45 bp, Homo sapiens
  source 1..45: mol_type = genomic DNA
tgtgtggtgaacatcgccggaggaggaagctacatacctacattt

SEQ ID NO: 1157 - DNA, 45 bp, Homo sapiens
  source 1..45: mol_type = genomic DNA
tgtgtggtgaacctcgcagggggaggaagctacatacctacattt

SEQ ID NO: 1158 - DNA, 45 bp, Homo sapiens
  source 1..45: mol_type = genomic DNA
tgtgtggtgagcggggcatcaggaggaagctacatacctacattt

SEQ ID NO: 1159 - DNA, 45 bp, Homo sapiens
  source 1..45: mol_type = genomic DNA
tgtgtggtgagaggggcatcaggaggaagctacatacctacattt

SEQ ID NO: 1160 - DNA, 45 bp, Homo sapiens
  source 1..45: mol_type = genomic DNA
tgtgtggtgttccaggcctcaggaggaagctacatacctacattt

SEQ ID NO: 1161 - DNA, 45 bp, Homo sapiens
  source 1..45: mol_type = genomic DNA
tgtgtggtgactgctgcatcaggaggaagctacatacctacattt

SEQ ID NO: 1162 - DNA, 45 bp, Homo sapiens
  source 1..45: mol_type = genomic DNA
tgtgtggtgagagaagcctggggaggaagctacatacctacattt

SEQ ID NO: 1163 - DNA, 45 bp, Homo sapiens
  source 1..45: mol_type = genomic DNA
tgtgtggtgaactccgcgggaggaggaagctacatacctacattt

SEQ ID NO: 1164 - DNA, 45 bp, Homo sapiens
  source 1..45: mol_type = genomic DNA
tgtgtggtggtcctcgcatcaggaggaagctacatacctacattt

SEQ ID NO: 1165 - DNA, 45 bp, Homo sapiens
  source 1..45: mol_type = genomic DNA
tgtgtggtgaatctggcatcaggaggaagctacatacctacattt

SEQ ID NO: 1166 - DNA, 45 bp, Homo sapiens
  source 1..45: mol_type = genomic DNA
tgtgtggtgcgtttagcatcaggaggaagctacatacctacattt

SEQ ID NO: 1167 - DNA, 45 bp, Homo sapiens
  source 1..45: mol_type = genomic DNA
tgtgtggtgaacgtcgcggcaggaggaagctacatacctacattt

SEQ ID NO: 1168 - DNA, 45 bp, Homo sapiens
  source 1..45: mol_type = genomic DNA
tgtgtggtgaattcggcatcaggaggaagctacatacctacattt

SEQ ID NO: 1169 - DNA, 45 bp, Homo sapiens
  source 1..45: mol_type = genomic DNA
tgtgtggtgaacgaagcctcaggaggaagctacatacctacattt

SEQ ID NO: 1170 - DNA, 45 bp, Homo sapiens
  source 1..45: mol_type = genomic DNA
tgtgtggtgaggaaggcatcaggaggaagctacatacctacattt

SEQ ID NO: 1171 - DNA, 45 bp, Homo sapiens
  source 1..45: mol_type = genomic DNA
tgtgtggtgaccccggcatcaggaggaagctacatacctacattt

SEQ ID NO: 1172 - DNA, 45 bp, Homo sapiens
  source 1..45: mol_type = genomic DNA
tgtgtggtgaaagcggcagcaggaggaagctacatacctacattt

SEQ ID NO: 1173 - DNA, 45 bp, Homo sapiens
  source 1..45: mol_type = genomic DNA
tgtgtggtgaacgatgcatcaggaggaagctacatacctacattt

SEQ ID NO: 1174 - DNA, 45 bp, Homo sapiens
  source 1..45: mol_type = genomic DNA
tgtgtggtgaaccccgccctgggaggaagctacatacctacattt

SEQ ID NO: 1175 - DNA, 45 bp, Homo sapiens
  source 1..45: mol_type = genomic DNA
tgtgtggtgaagctcgcatcaggaggaagctacatacctacattt

SEQ ID NO: 1176 - DNA, 45 bp, Homo sapiens
  source 1..45: mol_type = genomic DNA
tgtgtggtgagacgggcatcaggaggaagctacatacctacattt

SEQ ID NO: 1177 - DNA, 45 bp, Homo sapiens
  source 1..45: mol_type = genomic DNA
tgtgtggtgaacgaagcgtcaggaggaagctacatacctacattt

SEQ ID NO: 1178 - DNA, 45 bp, Homo sapiens
  source 1..45: mol_type = genomic DNA
tgtgtggtggccggggcatcaggaggaagctacatacctacattt

SEQ ID NO: 1179 - DNA, 45 bp, Homo sapiens
  source 1..45: mol_type = genomic DNA
tgtgtggtgagcggagcatcaggaggaagctacatacctacattt

SEQ ID NO: 1180 - DNA, 45 bp, Homo sapiens
  source 1..45: mol_type = genomic DNA
tgtgtggtgaacgcggccccaggaggaagctacatacctacattt

SEQ ID NO: 1181 - DNA, 45 bp, Homo sapiens
  source 1..45: mol_type = genomic DNA
tgtgtggtgcgggccgcatcaggaggaagctacatacctacattt

SEQ ID NO: 1182 - DNA, 45 bp, Homo sapiens
  source 1..45: mol_type = genomic DNA
tgtgtggtgaacaccgcgtcaggaggaagctacatacctacattt

SEQ ID NO: 1183 - DNA, 45 bp, Homo sapiens
  source 1..45: mol_type = genomic DNA
tgtgtggtgaggcgagcatcaggaggaagctacatacctacattt

SEQ ID NO: 1184 - DNA, 45 bp, Homo sapiens
  source 1..45: mol_type = genomic DNA
tgtgtggtctccggggcatcaggaggaagctacatacctacattt

SEQ ID NO: 1185 - DNA, 45 bp, Homo sapiens
  source 1..45: mol_type = genomic DNA
tgtgtggtgaagactgcatcaggaggaagctacatacctacattt

SEQ ID NO: 1186 - DNA, 45 bp, Homo sapiens
  source 1..45: mol_type = genomic DNA
tgtgtggtgaccggggcatcaggaggaagctacatacctacattt

SEQ ID NO: 1187 - DNA, 45 bp, Homo sapiens
  source 1..45: mol_type = genomic DNA
tgtgtggtgaacccggcgtcaggaggaagctacatacctacattt

SEQ ID NO: 1188 - DNA, 45 bp, Homo sapiens
  source 1..45: mol_type = genomic DNA
tgtgtggtgaacaatgcaccaggaggaagctacatacctacattt

SEQ ID NO: 1189 - DNA, 45 bp, Homo sapiens
  source 1..45: mol_type = genomic DNA
tgtgtggtgactcctgcatcaggaggaagctacatacctacattt

SEQ ID NO: 1190 - DNA, 45 bp, Homo sapiens
  source 1..45: mol_type = genomic DNA
tgtgtggtgaatggggccgcaggaggaagctacatacctacattt

SEQ ID NO: 1191 - DNA, 45 bp, Homo sapiens
  source 1..45: mol_type = genomic DNA
tgtgtggtgaatcaggcatcaggaggaagctacatacctacattt

SEQ ID NO: 1192 - DNA, 45 bp, Homo sapiens
  source 1..45: mol_type = genomic DNA
tgtgtggtgagttttgcatcaggaggaagctacatacctacattt

SEQ ID NO: 1193 - DNA, 45 bp, Homo sapiens
  source 1..45: mol_type = genomic DNA
tgtgtggtgaaccccgcgacaggaggaagctacatacctacattt

SEQ ID NO: 1194 - DNA, 45 bp, Homo sapiens
  source 1..45: mol_type = genomic DNA
tgtgtggtccgcgaagcatcaggaggaagctacatacctacattt

SEQ ID NO: 1195 - DNA, 45 bp, Homo sapiens
  source 1..45: mol_type = genomic DNA
tgtgtggtgatctttgcatcaggaggaagctacatacctacattt

SEQ ID NO: 1196 - DNA, 45 bp, Homo sapiens
  source 1..45: mol_type = genomic DNA
tgtgtggtgacacacgcatcaggaggaagctacatacctacattt

SEQ ID NO: 1197 - DNA, 45 bp, Homo sapiens
  source 1..45: mol_type = genomic DNA
tgtgtggtcactcatgcatcaggaggaagctacatacctacattt

SEQ ID NO: 1198 - DNA, 45 bp, Homo sapiens
  source 1..45: mol_type = genomic DNA
tgtgtggtgaaggtagcatcaggaggaagctacatacctacattt

SEQ ID NO: 1199 - DNA, 45 bp, Homo sapiens
  source 1..45: mol_type = genomic DNA
tgtgtggtgaacatcgcaacaggaggaagctacatacctacattt

SEQ ID NO: 1200 - DNA, 45 bp, Homo sapiens
  source 1..45: mol_type = genomic DNA
tgtgtggtgttccaggcgaggggaggaagctacatacctacattt

SEQ ID NO: 1201 - DNA, 45 bp, Homo sapiens
  source 1..45: mol_type = genomic DNA
tgtgtggtgaatggagcatcaggaggaagctacatacctacattt

SEQ ID NO: 1202 - DNA, 45 bp, Homo sapiens
  source 1..45: mol_type = genomic DNA
tgtgtggtcccccttgcatcaggaggaagctacatacctacattt

SEQ ID NO: 1203 - DNA, 45 bp, Homo sapiens
  source 1..45: mol_type = genomic DNA
tgtgtggtgagtatggcatcaggaggaagctacatacctacattt

SEQ ID NO: 1204 - DNA, 45 bp, Homo sapiens
  source 1..45: mol_type = genomic DNA
tgtgtggtgaaaggtgcatcaggaggaagctacatacctacattt

SEQ ID NO: 1205 - DNA, 45 bp, Homo sapiens
  source 1..45: mol_type = genomic DNA
tgtgtggtgatcagtgccccaggaggaagctacatacctacattt